SEQUENCE LISTING (78 sequences)

SEQ ID NO: 1 - RNA, 63 nt, synthetic construct
  source 1..63: mol_type = other RNA
caaggtcaatgaggagatgtacatagaaatacaatcctgtgtacatcttctagcatgacccac

SEQ ID NO: 2 - RNA, 57 nt, synthetic construct
  source 1..57: mol_type = other RNA
caaggtcaatgaggagatgtacataatacaatgtgtacatcttctagcatgacccac

SEQ ID NO: 3 - RNA, 90 nt, synthetic construct
  source 1..90: mol_type = other RNA
cccaacctctgtctactcaccacagccccccagcatcactgtgaatgctgccatggatgctagagggctacaggaagagccagaagttgg

SEQ ID NO: 4 - RNA, 60 nt, synthetic construct
  source 1..60: mol_type = other RNA
ctcaccacagccccccagcatcactgtgaatgctgccatggatgctagagggctacagga

SEQ ID NO: 5 - RNA, 51 nt, synthetic construct
  source 1..51: mol_type = other RNA
aagtggcatatgcgacggccatggactggttcatagccgtctgttatgcct

SEQ ID NO: 6 - RNA, 45 nt, synthetic construct
  source 1..45: mol_type = other RNA
tggcatatgcgacggccatggactggttcatagccgtctgttatg

SEQ ID NO: 7 - RNA, 48 nt, synthetic construct
  source 1..48: mol_type = other RNA
cattaaggtgggtggaatagtatacaaagtatcccacctaccctgatg

SEQ ID NO: 8 - RNA, 48 nt, synthetic construct
  source 1..48: mol_type = other RNA
cattaaggtgggtggaatagtatacaaagtatcccacctaccccgatg

SEQ ID NO: 9 - RNA, 51 nt, synthetic construct
  source 1..51: mol_type = other RNA
tccgtttaggtgggtggaatagtaatacaaagtatcccacctacccagacg

SEQ ID NO: 10 - RNA, 35 nt, synthetic construct
  source 1..35: mol_type = other RNA
catttaggtgggtgggctaaccacctacccagatg

SEQ ID NO: 11 - RNA, 41 nt, synthetic construct
  source 1..41: mol_type = other RNA
catttaggtgggtggaatgctaaatcccacctacccagatg

SEQ ID NO: 12 - RNA, 36 nt, synthetic construct
  source 1..36: mol_type = other RNA
atgcgacggccatggactggttcatagccgtctgtt

SEQ ID NO: 13 - skipped

SEQ ID NO: 14 - RNA, 479 nt, synthetic construct
  source 1..479: mol_type = other RNA
ctcctcggtgcaacctatataaggctcacagtctgcgctcctggtacacgcgcttcaacttcggttggtgtgtgtcgaagaaacctgactgcgccctgaggagaacagcggagaaggtccaccgagcctggcgaaaggtccgctgagcgggctgtcgtccggagccactccgggctgcggagcacccagtggagaccgcgcctggctcaggtgtgggaccccatccttcctgtcttcgcagaggagtcctcgcgtgaaataagcgggttttgaaaacaaaaaaaagaaggagtggaagagggggccaggatccaggcctccatccccacagaagtgaagctacagctgggaggtctcctcccaccccaaccgtcaccctgggtcccgactgcccacctcctcctcctccccctccccccaacaacaacaacaacaacaactccaagcacaccggccataagagtgcgtgtgtccccaac

SEQ ID NO: 15 - RNA, 515 nt, synthetic construct
  source 1..515: mol_type = other RNA
atacctgtcatgtccttcaggatctctgccctcaaaatttattcctgttcagcttctcaatcagtgactgtgtgctaaattttaggctactgtatcttcaggccacctgaggcacatcctctctgaaacggctatggaaggttagggccactctggactggcacacatcctaaagcaccaaaagaccttcaacattttctgagagcaacagagtatttgccaataaatgatctctcatttttccaccttgactgccaatctaactaaaataattaataagtttactttccagccagtcctggaagtctgggttttacctgccaaaacctccatcaccatctaaattataggctgccaaatttgctgtttaacatttacagagaagctgatacaaacgcaggaaatgctgatttctttatggagggggagacgaggaggaggaggacatgacttttcttgcggtttcggtaccctctttttaaatcactggaggactgaggccttattaaggaagc

SEQ ID NO: 16 - RNA, 370 nt, synthetic construct
  source 1..370: mol_type = other RNA
ggatttggcgccccccctcctaggatctctctatataaggcaagcagtccggactcccgatacacgcgtttccaacttgagttggtgtgtgtcgaagaatcctgaccaactacgacctggggagagcagccaaccgagaaggtccaccgagcctcgcctaggtctgctgcgcgggctgcggtccggagccttctccggaccgcggtcacccagtggaccgggccgtcgcgggacccctcatccttcgtggcatcgcagaggaatcctcgtgtggaacaggcgggttttaagaaccaaaagacgccaaccacgagggtcccaggatccagggctccctccccaggggagtgaagcccctctcaccgcagcc

SEQ ID NO: 17 - RNA, 562 nt, synthetic construct
  source 1..562: mol_type = other RNA
catcctgcagtgctgcgtagaacctgtcatgtcctttgtagtctctgccctcaacttgatcctgtgcagcttctcaatctatgactgtgtggtactggaccttcagaggcgcacagagctcaagtcagttttcgtcttgactgccactttataagttgacaggcctgggttttacttgttaaaacctctcaccatctcaatcacaggctgccaagtgtctttacaaagaagctgatacaaacacaggccatgctgatttcttacagagggagagaagaggaagagaagaagaaagaggaggaagaggacatgacttgcccatatgctgggcaccttataaaggaagccagacttttcggtgcagtatggaaaggcttccgtgattctcttgctgcaccccacgaaacttcaccaccttcaaactccattttcacggttccgttaattttcaaggagcagcaactcgactggttctctgctacatgaaacacctcagcttgaaaaggaagtgctctctcagactgacttgtgagtgtgccttcacattctggtgcaaatcatg

SEQ ID NO: 18 - RNA, 745 nt, synthetic construct
  source 1..745: mol_type = other RNA
agcagtaacgtcgcggcgggttgcgggtaggactggacgccagagcagccgcgcagcgcctgaaccgctgcgggccgccgcggccgccccttcccaccctcgcctctgctgtctccagcctcgcttctccgactttcctgctcctctgctgccttcgtttctggtcctcggccgtcctcgccgcccgcccagaggagtccccgcgcccgccaagaagccgctttccgctggcccgcagccgccgcgacttcggcacagtttctccctctggctagtctcccaaacggccgctcctcgcccgcgggaagaccaggctgcgaccgcgaacgcccgatcctctccaggagccgcagcgagcgcccggcggccacgccccgcgaccacaccccggcggctctcggcccagcgcgcctgccttcgccgcccgccgtcgctcctcgcccgctgcacgacgacgcgacgcccctgctgcaggcggcggacccgaccggacccagacccagacgcaagatggcgacggccgcgtgactgcctcagcgtccccgagctcggctccgagtgcacctacggactgactgtgggggcagagaagggcgagatcaggactctgtctttgttaatcgtgactgcatgaaggtcgcctccctcgggcctacttggtgggagtgtctggtattgttctaaggccaggagcacggtgagccacagtctgttggtagaatttggcgtcttgatagttgagaaa

SEQ ID NO: 19 - RNA, 795 nt, synthetic construct
  source 1..795: mol_type = other RNA
gtgccaggaaaggcagctttagtgcagacctagatcacagctacttttcttgtccctgtggggtcttacagatgtgtctctgagtagtaaaggcttagccttgttctgttttgttgttttttggaggggaaggttagtcaggcctgagtattcatgtaacattctaaaattgtgccagcgagcaccgtgaacgactgcaatgcaagcgggtcttgctggctaaaatgccaggtaaagggttggttggacacagcgcttagtgcacgctgtcatcatggacatcataatcagttgtgaaaaacacgcgaacctatgacacttcttattccacactgaatgtgaaattgcatgttcagatgtttactacgaggcctggctcacaggaagtgttcagtaaaagtatgcactgttagattactgataacgcggatagatttttgtttaccataaattgttccagatttatattaatggaaggaagtgtgcatttattagctattactcaactttacaatgcaaacatcttatttctcatctttaaacatgtcgaccagtttaattgaaaagtattctgagactgcaaaatggggtgttaaaaaatactgcagttacggagctgtgtaaaccagtttctcattgcataagatacagatgtaaattgcatggagaggttgatatgcacctgtacagtaattcactcccccatttcacatctttgtcagagaatagttcttgttcatactgagtgttctaaatttgaagttatatatacaaattaaaatattttaaaaattc

SEQ ID NO: 20 - RNA, 562 nt, synthetic construct
  source 1..562: mol_type = other RNA
agctgcacgccggccgttgcgggagcgcgcggacataagcgctaggcagagctgcctccagccgagtttctcccccatcccgtgcttcctgccgcttgggttcccggtgctcggagtgccgcctctccccacgtagccaagccacggcctcgcgtgaccctcagcccgccggcgtgccggggagaggtgggaggagctgccacgccccgcgaccgcacccagccatctcccgcccggagtcatcagtcttgagcccaccttgctccctctctttaagcggtgacgccagacacagctgcagccagttcagccctccctgcccggaccaaagggggcgacattcgagtgataggcggagtgtcccgtggctcccgaccctgacttgggacccggaagggacaagaaggaactctggtgtcttttggtaattgtggttgcctgaagcttgtcgccccctggcctaccgggggaggagagcatgttgtattgtcctaggccctatagcctcgtgagccagtccaagcagtgtctgtagaactggcacttttaatagttgggtgaa

SEQ ID NO: 21 - RNA, 736 nt, synthetic construct
  source 1..736: mol_type = other RNA
gtcctaggactggccaggctgcttttgatatacctgtgacgttttagtaagcctgccactcaaagcaagcacccagcctcctcctggcttggttttgtttcctgagggatgggatggggaagtcaggcccgcatattcctgtaacattcacagaagtgtgtgattgtcaggagctgcaacggtaaaggtggtccttactgcctaaagtgaatgcttgatgccagccctcctggcgacatctctgacagctgtgcacaaacgtgaagctgtgacacttcctgtcaaggtggttgcgaagctgcaggttctgatgctaaccgcgcaccaggttcacagactgtacagtacaaactcttgtgtttgttactaacactgtggatggttttactgtaaattgttcccaatttgtatcgatggaaagaatgtgattctattagaagatattactcatcttgaaaaatgcggatttctaattcctcacggctcaacgctggaatcagtttccctgaaaaggatcctcggactaaaagtggcatgttagaaaaaaccaggcctttaccgagctgcacaatcagttcctcacggtggaagacagaagtcctagggggagtggccgtggccagtctacacttcccccacttcaaagactagtcatttaataattggtcagcgctaagggttctaagtttgaagttattgaatagatgtatacaaataaaaatttgaaaattctcaa

SEQ ID NO: 22 - RNA, 275 nt, synthetic construct
  source 1..275: mol_type = other RNA
gcactggcccacgtgctgcgcgagcgagggagagccacagtctgagcgaacgtccgcgctgggagccaggggtgcccgacccccgtccgccgccgccgccgccgccgcgcatagcccccggagagccctctggggaccccgaccagaagggaccttgccctgggagaaggctgtggagacctgggccttctgcgatcaccctaggagttgatccagatatgtgcctcacgccctgatcactccccccaaattagtatccgcagagattcgaggac

SEQ ID NO: 23 - RNA, 2015 nt, synthetic construct
  source 1..2015: mol_type = other RNA
gctcgggagaacagggcaacatttcctaccacaggccaaggagacaaaagagatattggaaggaggggaaagagaagcccagacaaacagcagatgagttgagtggggcagagggacagggcagccagaccaaggccaagccttctcacccttggccagctggaagggacttcagcaaccaagaccacctggcaacaggctcagtgggggtcaggtccaggtccccgaagaggtgctggagaggaaagcagggagccactgcatccagcacatggggtgcctgggcctcagatggggaccccaaagaagcagaagctgaagaaggtacggctgggggttctgtcctgctcatccaaccacccctaaatacccaccctgtggactttgagctgaacatgcccactggcccccaggccacatgggacctggaggagcctacctggggcctgcccctgccagcaggtgccagggctggtgaggaagagctggggggcagaggtaaagccctgcaggggaggccacagggtccatcccgtcttcaggatcatctacactgcactaggggagccccaggaaggcagcaccctggaggccctgtgccagtgaggacaggagaccctaaggccccgggagcccagtgccagccagaggttgtgcaggcaaggagaccaaagattgatgagaagacccccagcaggggtactgggtacccggcaggccagtgccctcacagttgacttggaccagggtggctgtgaagggaagtctttgttgcaaaggaggaggaaaagggaggacttggtagggttttgtttcttctgcttgtttctgtacagggccaccagactcctggagagatcaagcaaggagaacctggggctgccatggccaaagcaactcaacagatgccaatgccaattccaaggccagccacaaccctgccaccttggggaatccagcctggaggcatcccctaagcagccagccatggcctgggtggaggcacctgaagacgtctgtcccaaactcccccagccctgagctgggagatgacagggggaaagaggccctctcaagggtgccagatgcctgggtctcccaagaggggtcccccaactcactgttcccgggacaggctgccccctgttccaggaagctcatcctcacctgtgtaggcccctgtagtgacccacgcgtccagcagacgcccacccaccgctagccgttgttcctgtgcaaagtagtgtgctatgcacccacccaggtggccgcctctgggcccaaggcacatgctgtgagcttcctgtgagcccaggctctgctcactgctgtcccgcgtcatgagcaccacctctgctttccctgtgtagatctaggccagtggctgcttgttcttgtggagctgtgtgtgttcttctctgagcagctcctccccggagtcccccagcacagtcccaggagatgacaggaaggaagcaccagggcaaggcggacgctcaccctgtgaccacgatggtgaccgtgactgtgggaggaagaactggacccaggacggagcggggctgccctgcctgaggctcccgaggagctttgtgctttggtgttccacccctgttgttactcatgactcagtttccttaacctggtagggtgttccctgctgtgttttccagtgtcctgtgactgtcctgtgcgggccatagggcagggccctgccccagcagatgggcttgggaggggactccctaaagccagtggacactgccagagtctaccttcctggcaagaggcagaccccggggccctcaggaaggagggagttggcagcgggggctgcagcaggagtaggagcagatgaggcgtcttgccaggaacctcaggaggagggggcccgggacctgtgtgggacctgtgtcctgtggtggccgtttgcagtttctctctgtgttgtgattcccttctcttcaacgttttcagtacgtgtttctcttcaataaacttcattcagtgttcca

SEQ ID NO: 24 - RNA, 35 nt, synthetic construct
  source 1..35: mol_type = other RNA
ctccccccacattagagtctcttgaagttggggcc

SEQ ID NO: 25 - RNA, 1968 nt, synthetic construct
  source 1..1968: mol_type = other RNA
gctccagggaacagggtaacctttgttcctgtaagccaaggagaaaaaagagatatttgaaggagggggaagaacagctcaaaaaaatcgcagagaaccttagaaaggcacaggggcaatgtaatcagtcagaagtcaaggcccctaagccaataccagctggagaaaattctaacaaacaaaaatccattgcattcaactcaataatgaccacacgcaagacctctaagattcaccagagaaggaagcctcaaggaactgctacagatgtgtggggccacaagaactcagatgggggtgtcagcaaagtgaaggcaacttcaagtccatatggggtgtggaggagcctgccacagggttgtcttgggcagaggacaccagtgcctgggaacaagcacggctaggcagggagacagtacccaggagaggaggccgcaggatccagcctgtcttcaggatcatctacacagctctaggggagccccatgagggcagcacccttgagtcctttcgtgagtgaactccccccgcccctgtgaaacctgcaagcctcaggacccaaaggacccgggaggcaagtgccaacacaaggcagtttccttcttgacaccctgacaggacactagtgcctgagaaaagccccaggctggggcacatggcaccacagggttaatgccctcagctggcctgagagcctgggagtgtgcaggacaggttttgtgtgagcaaacaggagcagcagaaagaggactctgtgtgtggggcgggggcaggcagggggcgggggggggggggagagtttgttctgctctcttttgtccagggctgctggagaattccagagagcaagcagggacagccaggtgaatacaactctgccaggcagctgccaaggctggccccaaaccctgccgacctggagggtggcctggcagctttgccgaggtgccatcctccatctgccacccataaccaaacaggtggacatcacagaaaaggccatctcaagggttctgccaactgcctggctctcccaacacaaatgccccttcctcttccctttcccctctcccccttcccctgccactgctgtccagaggcaggctgccccctgctctggaaccccgcccccttatctgttaacctcatagtgacccagctgtccagtgagtcccttcccccatcacgtgcttacccaccatcagctgctcctgtataaagctgtgagtgagggcaactggaggccctgactacctacctggtcagccacttctacaccctggcacgtgttgtgagcttctgtgccagcctaggctatgctagctgctctcttacccacctggtgttgtgaactccttgtcagctttcctagtgtacatctaggccagctgcctgctgcttgttctcttgcagatgtgtgtgtgtgcgcgtgtgttctaacctgatcagatgctccccggagttcctgagctccgtctggaagccagcaggaaccatggatgttgacaagagactggctgatgctcatcttacgattgatttcagtctgggacaggaagacctgaactgggcatgggtagggcctggggactgggctgtgctgatcccctcctgttgttccttgtgactatcattccttgggttggtggggtattccttgctttgctttgtagtttcttgttactatcctgtgccagaatagggcaaggccaccctacagtggtaatcccttgtgagagttcctaaaagagtgaggaaattttaagaggtagctcttgggcctttgggaagggaaaaatagcagagggattccatggtggtgggcagacaaggcccatctgcgaagctccaggagggtggcccagaacctggtgcttccagtggctgtggagaggtcctgtgctgctaaccccttctcttcaatagtttcagcaagtgtttctgttcaataaacttcataaatgttcc

SEQ ID NO: 26 - RNA, 363 nt, synthetic construct
  source 1..363: mol_type = other RNA
gcgcagcttccccgagcgagaccaaaacaggtggaatccgggctggagccggagctccggcggcgcgggtggcggcacgtccctccagacagtaccacaggcacctggagtaccggcatcggtcgctgtggcccccgagtgtccgtcagagcctaggggagcctgccctcccgcgcctcgtcggggcccggccaggcaccttggccgccggcgcacggacgcgggcacgagcactagatcacggctgctggacctcggcacgttgacaagatttctctggggtaccgcggaggattactttgaatttcggtggtcgcctgtggtctggcatatttagaacttaagtctattatttcgggcacc

SEQ ID NO: 27 - RNA, 1046 nt, synthetic construct
  source 1..1046: mol_type = other RNA
ggcagctgaggaggaggaggcccttctccaggcaatattggaaggtaatttcacctgagtctcagggaaccacgaagggatatggcaatgagtagagcatgaaggtagaacagtctatatactcttgtgacacatacaatccctaccttgtgctgccaagtaactcatttttgtgcaattctcagtataagccctttgtcgtttctgtgcctatttaaagtctcctaaaggtgtaattgactaggaaggatgtagttctacactgccatttacctatttaaattcatccttgtgaatatctttgttgttgttgttgagacagagtctcgctctgtcacccaggctggagtgcagtggcgtgatcttggctcactgcatcctccgccttccaggtttaagctattctcctgcctcagttgcccgagtagctgggactacaggcatgtgccaccacgcccagctaagttttgcattatcagtagagacggggtttcaccatgttggccaggctagtttcgaactcctgacctcaagtgatccacctgcttcggcctctgaaagtgctgggattacaggcgtgagacactgcgcccagcctcatccctgtgaatttcttattgtacacaagttctttcactatctgtgtgcagtgctctgaggggacagacaaggcttgggtgtatatgccaaccagatctcattgaagtatcagcttgtttggtactaggtgcaagtgtagcatgtcacatgtgaccatgttggatcattgacaatttttaggtatgtactgacctacatttatgatgaagatcctgagcggaggttaagatattaagttattttccatatgaatcagaattatattgattctgtgcaatcaaaacaaaaggcagaatagaatgctgagattggttaagtttgcaatgaccatcttgaaccacagatttctgctatgtgtcatcaaaacatctagttctgagtaacattttcacgattgttataaaattataggtgtgaacttctaaaataaaggaatgctaataaaa

SEQ ID NO: 28 - RNA, 269 nt, synthetic construct
  source 1..269: mol_type = other RNA
gcttcgcgccgcctccagaggcggttgtctgttccctgaggcgtttgctgcggtcgttcttctgccctcggccgggaggcgggcgcggggagcccgggtctctcctaaaccccgcaaaggtcagacgtcctctccgggacccaagcgatgtatctacgggcaggctcccggacctctgcgtgttaaagagacgagcacgcacatcactgtaagcggcggcggcggcggcggcgccctggtcgaattagaatttaaatactctgagcacc

SEQ ID NO: 29 - RNA, 2400 nt, synthetic construct
  source 1..2400: mol_type = other RNA
cccaagacccaaggctgtcatgagcagaacaagatggaagagctcattgcagagatcagcccatccactctttatgctgccaaagtaactcgccctttgtcgtgttgtttactgaatgtctccccaatgttttactgataaaaatgatgtcattctacactgataattatttaaatcaaaagaccattctgctagtacagtgtcccagcaagtcaaaagtttatatgcatgtttaatagtcatatgtgatggtgctatcagttatcaatagttttcaggtgttgcccacatttatgctgaagaccctgagccaaggctggttaagatatttagtttcttttattaaaatcagttatcgattctgtgcaatcaaaaagcacaactgaatgctgtggtcagttctacaactagttttaggtttgtgactatctgatatgttgccaaaacgtctgcttctaaatatcattttcttggagtaataaagaaatgataataaaatatttgatctttgtatacattgtaatactacatttgaaggaaaataattttttggctttgtttttaagacagtttctctgtgcagctttagctgtgctggaacccactctatagacccagggtagtctcaaactcagaaatgtgcctcctgagtgctggaattaaaggcctgcacaactacactaggctgaacaaaagtttttatagtgtgaatctgaaagccactgaaattacagacttagttctttgaactcttggcacctaggggataggaacttgcttgtttgacaggaaccagactataaacagattgtggcgagtagtaggcccatcaatattgaattttgagggagaaagggaagatgaaggtttgtgtttttccaaccctggtgctacacacctataaccccagtgcttaacaagtaaagccaaagtcaggaagactggcctctcaaggccagccagaactacagagggaggccctacctccaaagagaaaactacatacagggaaactaagatcacctgggaaatggatgtatgcccaagacgaagcaggcagtaatgagagctgttctggttcttgcaaagctgctgttctctctactaagggagggaactacacaaagaatcaaggccagactgtcaccgtgtgtacatgtagtcttgggagtgtgatagattactctgagttaggaatacatggaaagtttccttatgttctccactgcagaatggcatgtggactcttgagagctgtgattctgctcatgtttgtatagtggcttaagatacattatgcactgtcctgcaggtcactcatttgctgcctcggtgagaaactttacattttgtatagacacacaagcattaataaatgccacgcctcttctgtatgtgagaccctaagctgagggggtaaactcgacaggataatccctgctttgaaggtttattcagactcagtattaatggctgacattggatgtatccttggccagcgactaagtactagctgaagaagaagctgtctcttcccacctcccattttatgaataagatgaggcacagaaaaataacttgtctgtggttttacaggtagtaatgtggctagcccctggctccaggtctttttattctgggtagatcataggctcagagatctttaggtgctggtctttgacgcatatatatatatatatatcccataacatttggcagtggtggcaacagacaggtggagaagagtgccacctagtgttatagctctaggggcctaggatgctagacccttaattaagcattatagatcttgtggaagcctctagtaaccccaccctttgctctaaaatactaagttattgggtagacctcattacaagtattatagctgttgtatagacttgggaagttattattccttcgccttgccagtgttaaatcgtacaggtctcggggtgtctgagacttgtaaaaatcacagaagctctctgaaacctccagtgacaagggcctgctggttttcatttccagctctttacagccttctctcctttatctctgcctcttcggttgtcacaacagccagccacacagccattttccagctctgcctgccactccactgtctgctgttgctgccactctcctggctttatattgccatcttagcctcacaactaagaagtaagctagaaataggaaaggagggctggagaggtggctcctcgggtaagagcaccgactgctcttccagaggtcctgagttcaaatcccagcaaccacatggtggctcacaaccatctgtaatgggatctgatgctgccttctggtgtgactgaaaacagttacagtgtactcataaataaaataaatctttttaaaaaatggcaaggaaacact

SEQ ID NO: 30 - RNA, 439 nt, synthetic construct
  source 1..439: mol_type = other RNA
tctcattgcattgcgcagagctcagccatctttctcatccgtcaggcattcctgagaaagagggccactctacctctcttgctgccagccttcactccagcaaggagggtgctgggtgacctgagcccacatagccccgagtgagcagtgaggctgtctcctgccctcttctgcctggagggctcgtcagtgtccccaggtgtcaggccctgcctcctgacgttggccttttcactacaggcacccgaagcacacacagcagagtcctctggactttgaggaagaacccacagcaggaggaagtcagcagggagtggctgtgtgaaacctgggaccacttctgccttcctacgtggcagtggctcagagttatttgagtgctgtcaaactgagctgattgctgccctagtattagatcagtccatagaaagtgggagca

SEQ ID NO: 31 - RNA, 1432 nt, synthetic construct
  source 1..1432: mol_type = other RNA
gctcagaagaccgaagcactccttcccctggcaggcagcagggacatttgaacaaggggaaggggcagcttacactaacagggaacttgactggggcagagggacagggaagccaggtcgagatcaagcccccttactttccaccagctggaagggacttcaccaaccaagaccaaatagcatctggttcagtgggggccgggcccatagcccctaacaggtacaggagacacaagcggggaaccactacacttagcacatggggtgcctgggcttcagatggggaccccagcgaagcaggggctgaagaaggtgtggctgggggctctggcctggtcctctggacacccccaaatatctaccctgtggactttgagctgagcatgcccactggcacccaggccacgtgggacctggaggagcctgcctggggcctgcctctgccagaaggagccagggctgctgaggaagagctgtggggcagaagtgaagccccgcaggggaggccacagggtccagcctgtctttaggatcatctacactgcatgaggggagccccaggaaggcagcattcataagcccctgcaccagtgaggaagagaaagaggaggagaattaagaagaggatgatcgggaggaggaagaggagaataaggaggagcaggaggaagagggggaggagaaggaggaagagggggaggaggaagaggggaaggaggaggaagaagtgtaagaagagaagagaggatgtgttgggggtgggtagttgtttttggtttgcgtcatcatcaggcccgggctctgctcactgctgttccgtgtcaagaactccacctctgctttgccggtgtagatctcggccagtggctgcttgttctcttggggatgtgtgtgtttttatctgagcagctcctttccagagtaccccagcgcagttccaggagacggtgggaaggaggcatggcagacgctcaccttgtgactgtgacctcgactgcaggaggaccaaccagacacgggaaggagcagggctgcctgaggctccccaggaagctttgtgctttggcgttccacccctgctgttactcgtgactcagtttcctcaacttggtggggtgttccctgctatgttttccagcatcctgtgactgtcctgtgcagtctatagggcagggccctgccccagcgggtgggctcaggagggggttccctgaagcgagtacacattgccagagcccaccatcctggcaagaggtggatcctggggccctatggaaggagggatgtggtggggggggccacactgagggaggggccggggacttgtgacctgtagtggctgtttgcagtttctctctgtgttagattcccttttcttcagcagtttcagttcatgtttctcttcagtaaattttgttcagtgttccaaaaa

SEQ ID NO: 32 - RNA, 29 nt, synthetic construct
  source 1..29: mol_type = other RNA
gttaggtctgctgatagagggagggaaca

SEQ ID NO: 33 - RNA, 404 nt, synthetic construct
  source 1..404: mol_type = other RNA
actccccccgctcctgtgaaacctgcaagcctcaggacccaaaggacccgggagatgtgtgtgctaacctgagttcctgagcctggttctggaaccatggatgttgacaaggctgatgacttcaacttgggactatgtgtgatattcctctcctgttattacttataattttactttctagggttggtggggtgttgcatgctatggttccaccaattccctgtgcctggactgtgtatgctataagggagaactacactgtagtgagtgatctcttgagatagcagaaggccctgtgtgagcagagcctatggcttctagtgaccactcaaagtttctgttatgtttttaatggttttaatacaatttttgttgaataaatctcatgaatgttctggctaaaa

SEQ ID NO: 34 - RNA, 362 nt, synthetic construct
  source 1..362: mol_type = other RNA
acacccccccagcactcggcacagcctcgctgagcagccttcagcagcagggtgtggtggggagcctcggagagtctggggttccatcctggcccagcctcacaccagctgagagacgaccgactgagcttcgaggacaggaagccaccggcatcactaagccaccggcatcacttcatccccagcctcacgcttgggactgggcccggggaagcaagcagcgaggatcgagtggcacaggacgggaggagatccacttgaacgcttgcagccaaggttctgatctccacggtcccagctactgactggacgccatcacaaccttaccaatcttcagaatacactcctttccatccgacgaa

SEQ ID NO: 35 - RNA, 959 nt, synthetic construct
  source 1..959: mol_type = other RNA
agacgcctccctgtctccagcaaagagtcccctctatttctcaccccaacacctcccgacgtcccctggcctccccgcgcctcagcctgcttcgcttcccttcctgccactcctgacccaagaggaacccacttgtgaagggcaacaacttcttcaactcatcaaccagcaatgccacctgtgctaacggactacccagtcctcagagacccagcgccagccctgggctacgagttctccgttctccaggtcccaagcccatgcccgggagtgacatccatgagggagaaagcagtggtgagcaggaagtgaccccatgacgcgtggccagatgaagaagcaggcacagcaggcgcagccaagcaagcaggtgacggggccccccacccggcctggtgatggccaccctgggacactgacctcgggtgctccactgacttcttctacctccactgccggtgctcctgctcccctgagctcccgcccccgccccccaacccccatcccccaacctcccgacatctgatgatgtggtgcctccctcacccccatcacaccctgcaagcaaacccacccccacccccaccacatctgcttcagcttttttgaccccgccttcctcttgccctgagctctggacttacctggacaaagccgctctttggggcatccggacgtaccagtgccacttaccagacttgcacagcaaagagggcccctgcatgccttggggcgcctttccctcccaacactgaacactggacagtgcaggggtgcaggggcccctcatacagggatggagtcccagaaggcacacagacacccaggggaccatgccgagtcacagagaggctctggccacaccaccgctttgcaaggggagtagacccttccccaccccccgagctttctcaccccccaatctttttgcactcctcaaataaagcaaactaaaagac

SEQ ID NO: 36 - RNA, 147 nt, synthetic construct
  source 1..147: mol_type = other RNA
gctcagaggcaatcaaggagctaacgtgaccaagtctcgctctcgggcaggcgctaacagtggttttggtctccacagttgcggtctctgaccacacacatcacaatcttaccagtcttcagagcacactcctttccaaccgacgac

SEQ ID NO: 37 - RNA, 976 nt, synthetic construct
  source 1..976: mol_type = other RNA
aaacacaccaagtcactgtcattttcccttgtgcctcggccagcttcgctcagatccagcacaccctcttgctactcctaacccaaggggaacccacttgtgaagggccacaacttcatcaaccagcaacatcatctgtgctaaagaactagccaactatcagcgtcccagaaccagccaagactgtgtaaccctaactgtcctaactgtccgggccccaggcccacaaaagggagtgagaccaatgaggagagagcagtgatgagcaacagtgaccccgtcaccccaggccagatgcagagcagatacagcaggtgcagccaagcaagcagatgccagtgaccccaccaccaccccctgagtggcaggcggccacctgtgcatcctgggacactgaccttgggtgctccacggacttctacctctacaccggtgctccgactcccctggacacgcccctactccctcccagtctcccaaggtgctgtggttccatgctttccacaaccccagcccaccctgcaagcaagtccacccccccccccacaacccacccacccactaccggtctggtccggctccattgacacctcatctgaccctgaactctgatcttacctgggcagaactgctccaaggagcattcgacgtaccagtgtgacttaccagatggactgatccaccctgaaggtccctgagactcagaggacccaagaaagggtggtagcaaccaagtggggctcccttttccctccccaaatggacatatggacagtgcaggggacagagaccttccataaaggagggagcccccaagagacactggtgaagccccagatacccagagtactgtgccaaggagccaagagtgctctgaccagacccactgctcactgccacaggaaggagaggtccttccattgctttcccaactcatacctgcttttgccccatgaataaagagaagaaaagatta

SEQ ID NO: 38 - RNA, 473 nt, synthetic construct
  source 1..473: mol_type = other RNA
ggcgctgcctcgtctctgctacccctggttgggcggccctgcgaagcagctccttcgggcagccccgggtcgcttagcggccaaggaggcttcagttctttgccgcctgcaaggcggagaccagaaggcggaatccacagctggcgacgcgggagcatctgctgtccaccagcggagcacaggccatcaaagccgcatctgaacttgaattctgtgcagctgattgcagagctggacccggatctgcgaccccctgtggacagaggttgaccgtaccccggagaggagctttctcacggagggcactggttgcagaggctggaagtgaaataaagacgcgctcttgtttcagagttcgtcccctgctgagataggaaggcagagccacctcctctcctctcccacctgcagattaagcttttctaaaaagcctaggcatcttcttatattcagataccctatcgtcgtcagtc

SEQ ID NO: 39 - RNA, 516 nt, synthetic construct
  source 1..516: mol_type = other RNA
agggaccacccccaggtttcagtgaacccttacctatattcagcatccagtagtgggaaaactggggtgggggtgggggtgggacttctaactgcatgaattaatccacaaagcggctatcttttggggtggagtagaaagggtcttggataccagcacattggagggagatagcctgacctctgtccttgctccttctccctgcagcctacgggtctgttttctgtgtgtgcccatttccttgacagctttattctttgtgaaagtggtataatttattgttaaatatttgaacaataaaaaaggtacaaaaagtgaagtacaaattacccaaatctctccacccttatataatcattgtcaaccctttgatgagtgatatttccctatacctatgtacccagatagatatatgcatagataaaagtgatgaaatataagtgctgttctatctgtattttttcaccaaacaatatatgttgtgagcttctatgtcaataaatatatatatcagca

SEQ ID NO: 40 - RNA, 462 nt, synthetic construct
  source 1..462: mol_type = other RNA
tgattggctccgctggccagctcgtcacactcttttgtgtcagtaggctgctgataaaagctttgcagctgccttggaaactgcgctattcgaatccggttacctgtgggcagaatccatagctgaagacaccacagcagcctctggctaccagtggatcacagtagcagcccatcaaagcggaatctgaacttgaatttggtgcagctggttgtagagctggtcttgagcatcaggatctgagacccccagtcgatcgaggtccatcaactggaccctggaggcaggctttctcacgggagggaggacacaggttgcagaagctggaaatactccagtttcccagctgatccccctactgagattggagactgctgcactgctgaagataaagcgtttttgttttgtttctaaaacgccttgaacttcgtgtattaagatatcctatcactgctgtcagac

SEQ ID NO: 41 - RNA, 511 nt, synthetic construct
  source 1..511: mol_type = other RNA
ccagccttaaattaacccttacccgtattaaatgtttaacggtggaaaagttgtggggacagatttctaattgcatgaacaaatccacaaagtggctttctttgggggtggagaaagggtcccgaataccagtacagtaaagagaggtggcctgacctatgatcgtgttcccattccccactgcttaacagtctattttctgtgtgaacctctctccttggtggctttgttctctttgtgaaagtggtattgttcattgttcaatcttcaagtaagaaaattattaaaaagtacaaattacccagacctctctaccttgtgaaacaattgtcagccctttggtgcctatccttctaaatatttctctatatctgtgttcctagattagaaatatgtatagacgaaagtgatcaaatagaagtgttgttctatatgctgtattttttcaccaaaacgtatgttgtggccttctttgtcaataaatatatacatatatgtcagcatctacttt

SEQ ID NO: 42 - RNA, 195 nt, synthetic construct
  source 1..195: mol_type = other RNA
acagcactgcctgcctctgccccagcagtcagccagccgaccgcgcctgctccctcctgcttgcccaaggccgggcaagtcatccccactctgcttcgagaggccccgttttccagcgtgattgcgccgacactgctctgtgggtttctcttcttggcgtgggttgctgctgaggttccagaggagagcagcagg

SEQ ID NO: 43 - RNA, 563 nt, synthetic construct
  source 1..563: mol_type = other RNA
gaagccaccttttctttaacctcctaaatattggtgggaagacccaccgctgtggggggggttgcatatcctcatgggggtcactgggcttggccagtctgcttatcaactcttgctcttctctcccctttgcctccctctgcaggggccttaatctgcccctggtaggggaggcttccactgaacaggcacaggtgagggagagcaggctggcttagagggacagggtccccatggtcatcaagctgctgttgatgacaaagactcaaaggctggaagagctcccaaggaagctagaaatgcttgtctttgaaagaactgtgggaccccttcagattccctgaggtatggcttggtcactctcaggtcctcaaagcctgtcttagttgggctgggtcctagctgcagggtctttgtgagggtcacagttgctctgggacacctccctgaagagcctttccacctgtacaatcgtattttctttctgtcatttgctttgaagcccattgtgccttatgccaataattcaattgctgcaaacaccaataaagattgattcatgg

SEQ ID NO: 44 - RNA, 189 nt, synthetic construct
  source 1..189: mol_type = other RNA
acagcctgcctctgtgcccagcagccagccagacactttgtaccggctcccttcaactcgtccaggcagggcaagaacacggcccagccgacagagccctcgctctccagcgtgattgcgcccacactcttctgtgcgtttctttacttggcttgtgttactgctgaacttccagaggtgagcagaagg

SEQ ID NO: 45 - RNA, 509 nt, synthetic construct
  source 1..509: mol_type = other RNA
gaaaccccatttcttgttcccagcattggtagggggactttgtgttggggggagcagatgtcctggggggtatcatccggcctagccagtctttacaccggttctcagtttccctccttctacaggggccttgctttgcctttgtttggggagggaggccagcttggtggcctaaagcagtgtccccaaggtctgcaaagacttccaaggctggcaggagcttctgaggaagccaggaatgtcaatcttgagagaggacccttttagatcccctgaagtatggctcagtcactttcacgtccccaagcctgctgagctgagcctggtcttggctaagaccctcacaatccagatgcttggaggagactggcagctgctctgggagtcctccctgagtcctcccacctgcacaaggatgctccctcctgtcctgtcacttgccttgaatctcatggagcctgtatcaataattcaattatttcaaaacaccaataaagatctgttcatgg

SEQ ID NO: 46 - RNA, 208 nt, synthetic construct
  source 1..208: mol_type = other RNA
gcagagctcgggcgcggcgtcctccctccgcagcagccgagccggacctgcctccccgggcgtgctccgccggccccgccgccggcccgcagcgacagacaggcgctccccgcagctccgcacgggacccaggccgccggaccccagcgccggaccaccctctgtccgccccgaggagtttgccgcctgccggagcacctgcgcacag

SEQ ID NO: 47 - RNA, 1801 nt, synthetic construct
  source 1..1801: mol_type = other RNA
agggcatcccggagcccccagcctgcccactacatccagcctgtggctttgcccaccaggacttttgagctggggctgactcctgcaggggaagccctggtccagctgggtgccccctcgagctccgggcggactcgcacacactcgtgtcatccagatgtgagcaccgcacccagcggcaaagagccctcccccctgcagggctccacccatcaccctccctccgtctgtctttccggcctggaccccaccctccacactctcaggccatcacagaacaccccagcttcctcattctgctacaacacccaggccctctggacatccagaaaaccaagtgtccggatggcaggggccagcggccaccaagctcatgggacacccagagcagaagctagggcagagccaatgctgagggagcctcgacttccggcgccgccgccctctcccggcatccgcagagccagctgacgccctccctgcctcccagggcagctggccagcctcgggcagcgcggccccctcctcccaggggagagtagaagtcgcacacgcagcagagcagacctgatgtcccggtgcttcctggcccctcagctccagtgattcacgcccgcctggagaagaatcagagctcagctcatgactcacccatggcaggcggagggtcccagaggggctgagtcctcaaatccggctgaggcagcagctggcaccatcagagccaggagagtgacaacaggtaacggagcaccacccttccacccagaccccaccatcagctgtcccggccaaatgagctcctccccagaccccagacaccctgcggcccaggccactccccagtgttcgggctcgctgggaggctctgacggggtcggggggcctctgccttggaagaccagcgccatccaccggccccagcccctccccatggagccctggctactgtcctgcgtggtccttcactgcccactctcctgttctttcaggtctcaaggttcccacaaagtctttgctgctgtgctgggcaccacccacccctcaccttgcaggctgcctgcgtgggaggcgaagtcccaggacagcccagaggggggctacagagaggagtcggctgcagcagagggcaggagccccagcttagccctgagcgccagcgcgaggaccagggcctgccactaagcccgccccgctggccgccagctgcccgtccccagagccactgcagcaggagtcgggccctgcctccctcccagcagggaaaccccgcccgctgccaggccatcctctctgccagaggctttcatgagccccaaggctggggccacagctcctacccctgcccagcagccctgagctcagctgcaggaaggacatcccagaagccatggctcctggggcgcttccaggcattctgccctgccccgacaccagaaccctggtgctggtgggccactagcgtctgcagcctaagcaggtgctggctcagggttcatcgttctgccttgtccactgggggaccagccctgcagaccactctgacaagtcttcagcccacaccctgccagccccacagattttatttttgcacataagccataaccaatcctcaaggctggcacaggctttggggaagccctggagcctgtgaagaccctggaaacctcatgaggctgtggccaacccctgccccttgccccacacagaccaggccttaaatgtcggtccaggccctgtgcaccttaccccagagacagactctttttgtaagattttgttaataaaacactgaaacttc

SEQ ID NO: 48 - RNA, 198 nt, synthetic construct
  source 1..198: mol_type = other RNA
gcagagctcaagcgagttctcccgcagccgcagtctctgggcctctctagcttcagcggcgacgagcctgccacactcgctaagctcctccggcaccgcacacctgccactgccgctgcagccgccggctctgctcccttccggcttctgcctcagaggagttcttagcctgttcggagccgcagcaccgacgaccag

SEQ ID NO: 49 - RNA, 1670 nt, synthetic construct
  source 1..1670: mol_type = other RNA
aggggccagcccagggtccccagcccgcctgccacacccagcctgtggcttttgccaactaggacttgagctggggctgactcccaaagggatgccctgtccagccagacatctactcacccactgacctggcctgactcacaactgccacacaaccatgctacatggactgaacatcaagaagcccctttcccatagggctcccacctgccgcctacccctcatctgtctgccctggtcctggcccccagccccattggcctcaccctctacactctcagaccatcacagaacacgatctggcttcctcattctgctccagtgtccagggctctttgggtaatcaagaaaccaagtgtctgaaaggcaacaaaaagtaggcaccaaaacccaggggacatcctagggcagatgctaaagcagagatgctgagggaacctcaacttccggggatgcagcccactcctgcagacacagcagatccagctggtaccctagctgccgcccagggcaaccggccagtcttgggcagcatagctcccctcccaggggtgagctgaagccacaaatgcagctgaagcagcagacctgacatcctggcacctcctggcccccagcagtgattcataccagtgaagaagagcagagctcagctccgtgactcagccatggcaggcggagggtccccgaggggctgagtcctcacacccagccgaggcagcggctggagcctacagagccaggagaatgacaccaggtctcaaggctgctgagaagtctttgctgccatgtctggaaagggcatcaccacaccaccagcaccagcaccatcctctcttctcctgaagctgcctacatgggttccaagacactttcaaggcagagaaaacaagattacagagaggaggtgcctggcagggggcagcaccccagctcagcccaagagctgaaggtgaagacaagccagcatgaaaccacgggtctgccatgatgcccgccccgctggccgctcactagctgcctgccattagcctctgcagcttgagcagggtctgtacccttggtcctcttggcacagagcccagctcgctgcatggcctttggctccccgaccagacccttgcaggagccttaaggcttgggcccctgcccagcctgatctttcctgctgtgccctgcctgccaggtcaagcccagtcccaggagaccccaggccttggctcctaggctgttccatgaacctccctgacctgcctggtgattgcccagctgaaacctcagccaagccccagctccaattaccttgtgctggtagctgcttgtgtctgcagtctgagtaggccttgttgcggttcctccatctgcctggtctattggtgttctgagaccaattccactgatgttctgacagatcctccaccctgtgcccctgccagcccccacaagtttatttttgcacaaaagccatgacccatactcatttggctggcatagggtgtgtaggtaggccctggggactagggagaccctggagatctcaagagagtgtggctatcccctattttcaccaagccttgaatatccagccaggctgtctgcccataccatcttacctcaaagacagatatatatctatatatgattttgttaataaaactatgaaacttatt

SEQ ID NO: 50 - RNA, 360 nt, synthetic construct
  source 1..360: mol_type = other RNA
ccctgccttgtctaccaaaccctcaggcaggatttgcgcgccgaagtgctccgcggagagcaggtgtagcgggaaataagtaactgatctcgtcgacatgatcgttaagggtgggcgcggttgcgtaatgctaagatagctctctatttgcaccagtggtctatcggtacgtaaggcccaacaatgacgcggatagaaggaaacactcatccgtgtcgacttggtccacacgcgtactccgcggaacatacttcggtgtcgagtggtaatagatgccggttgtggcagagtctgaggaatactcacgtagcaacagctttgaaagtagaagaaacctgcctgactgtcggcgagtgaacc

SEQ ID NO: 51 - RNA, 90 nt, synthetic construct
  source 1..90: mol_type = other RNA
accaagtcgacacggatgagtgtttccttctatccgcgtcattgttaggccttacgtaccgatagaccactggtgcaaatagagagctat

SEQ ID NO: 52 - RNA, 141 nt, synthetic construct
  source 1..141: mol_type = other RNA
accaagtcgacacggatgagtgtttccttctatccgcgtcattgttccgtttaggtgggtggaatagtaatacaaagtatcccacctacccagacgtgggccttacgtaccgatagaccactggtgcaaatagagagctat

SEQ ID NO: 53 - RNA, 129 nt, synthetic construct
  source 1..129: mol_type = other RNA
accaagtcgacacggatgagtgtttccttctatccgcgtcattgtaccatttaggtgggtgggctaaccacctacccagatgcctgggccttacgtaccgatagaccactggtgcaaatagagagctat

SEQ ID NO: 54 - RNA, 135 nt, synthetic construct
  source 1..135: mol_type = other RNA
accaagtcgacacggatgagtgtttccttctatccgcgtcattgtaccatttaggtgggtggaatgctaaatcccacctacccagatgcctgggccttacgtaccgatagaccactggtgcaaatagagagctat

SEQ ID NO: 55 - RNA, 126 nt, synthetic construct
  source 1..126: mol_type = other RNA
accaagtcgacacggatgagtgtttccttctatccgcgtcattgtatgcgacggccatggactggttcatagccgtctgtttgggccttacgtaccgatagaccactggtgcaaatagagagctat

SEQ ID NO: 56 - RNA, 135 nt, synthetic construct
  source 1..135: mol_type = other RNA
accaagtcgacacggatgagtgtttccttctatccgcgtcattgttggcatatgcgacggccatggactggttcatagccgtctgttatgtgggccttacgtaccgatagaccactggtgcaaatagagagctat

SEQ ID NO: 57 - RNA, 2855 nt, synthetic construct
  source 1..2855: mol_type = other RNA
tggatttatgttgtatagattatattgagacaaaattatctatttgtatatatacataacagggtaaattattcagttaagaaaaaataattttatgaactgcatgtataaatgaagtttatacagtacagtggttctacaatctatttattggacatatccatgacctgaaaggaaacagtcatttgcgcacaactttaaaagtctgcattacattcctcgataatgttgtggtttgttgccgttgccaagaattgaaaacaaaaagtttaaaaaaaataataataaattgcatgctgctttaattgtgaattgataataaactgtccctctttcagaaaacagattaaaaaaacaaaaaacaaaaaaaaaaaaacaaaaaacaaaaacaaaaattggaaccaaaacattccgtttacattttagacactaagtatcttcgttcttgttagtactctgttttactgctttcgacttctgatagcgttggaattaaaacaatgtcaaggtgctgttgtcattgctttactggcgtaagggacggggaatgggaggggtagatttctgtttgttttgtgttttattttgtttgtttgtttgttttgttttttagttccacccggagtagggatggagaaaatttcttcactatccattctggttgataaagcgttacatttgtatgttgtaaagatgtttgcaaaatccaatcagatgactggaaaacaaataaaaattaaggcaactgaataaaatgctcacactccactgcccatgatgtatctccctggtccccctcagctcactcttctggcatgggtcagggaaaattgcttttattggaaagaccagcatttgttcaaagcatactctttccctccctcctcccattttggtcccttctttttgttttgttttaagaaagaaaattaagttgcgcgctttaaaatattttactactgctacaaacagatgaacaatgtgtgtcattttatgacactcatggaaaacagtgatttttttttaccctaaagaaaaacaaataaaaataacccaaaatattctttttttaaaaggcataaatattgggtaaattgtaatatggcctaacagtgtttgcagataaaagttattgtatacacccagatacttagataagagcagggatccacactgccattgaaataggactgaatggccctgcggaggctaagtggagctgacatactatttcctggcagtgcaggaggaatttctgagtggccatcctaaggtctaggatggaggtggggaatggtacttgagacattcctaaaggaaggctcggaagcacccttcagagcaggctctggaatgatgtgtcaagttgcttaggccttctgctttaagtgcctacattacctaacagtgctcaagaggttctcgattggagaaccacactcaaatccatttatagcctccatcccatttctaaataattgtgtataaagttggattaacctggagcaactttggatccaaatatggcacagcaataatgatattaatgcagcatgatgggaaatgtttgctgtgaagagaattgatttgctttgagcttagacttcaggaagcctaggttttttatttttttatttttgagacattttggtaaaaggaaaaaaagaaaacaaacaaacaaacaaacaaaaccagaaaaagcatcaaaactcaggcagaatgagcaatgtctgaaagggctagaaaaacaagacatagcaaggtgctttcactgtgaaagagacaagaacacaggaggaaatattgcttcagtgaagagcacagacggctcctgccaatttattacaagagtcccgtctgtactttaccctttggggttagaagtcaagttggaagcctgaatgaatggacccaatgagaactagtgttaagcccatttccctagtcaggtttttttcaagcgtgaatgtgttagtggttactctcctgggttcctgagcatcagaaaaaaaaaaaaaaagaggcaaacaatcgcttcatcttaggagtggaaaggaaacagaagtggacgtccgctgtgactcagggagtgaagataccatcagcaaatagtttcttttttgttcattcgttcctttcgagttagcctgtcttttggaataccactgaatatgctgtttttgaaagacttcatgtagcatagattgttttgtgccgtttaccaaattaacctttgtcatcgttttttaacctattcaggaatgcttggaatatctgctctatgttaactttttgcagcttcattctgagagacattagtcaaacaaacaaaaggatccccatcacaatcttacagtactgcaagggccaggtctgttaatcggcttcacaggagacatcagcaattgtgtggtcagtggctggctctcttacccactaagatacatcatagctacatgttggtggtttatgttgacctgagatttatttgttaaaatctcttcttcgtttctgttcgttctggttctgttctgttctgttctgttctgttttggttttaaagtcttgctgtggtctcttgttggcagaaatgttttatgcatggcagcaggcctgttgcttttttatagtgattcccattgaaactgtaagtaaatgtctgtggccttgttctctctatggtaaagatattattcaccatgtaaaacaagaaaaaatatttattgtattttagtatatttatataattatgttattgaaaaaattggcattaaaacttaaccacatcagaagcctattgtaaatacaggttctatttaagtgtaccaattaacatataatatatgttttaaata

SEQ ID NO: 58 - RNA, 141 nt, synthetic construct
  source 1..141: mol_type = other RNA
caaacatctttacaacatacaaatgtaacgctttatcaaccagaatccgtttaggtgggtggaatagtaatacaaagtatcccacctacccagacgtggatagtgaagaaattttctccatccctactccgggtggaacta

SEQ ID NO: 59 - RNA, 129 nt, synthetic construct
  source 1..129: mol_type = other RNA
caaacatctttacaacatacaaatgtaacgctttatcaaccagaaaccatttaggtgggtgggctaaccacctacccagatgcctggatagtgaagaaattttctccatccctactccgggtggaacta

SEQ ID NO: 60 - RNA, 126 nt, synthetic construct
  source 1..126: mol_type = other RNA
caaacatctttacaacatacaaatgtaacgctttatcaaccagaaatgcgacggccatggactggttcatagccgtctgtttggatagtgaagaaattttctccatccctactccgggtggaacta

SEQ ID NO: 61 - RNA, 135 nt, synthetic construct
  source 1..135: mol_type = other RNA
caaacatctttacaacatacaaatgtaacgctttatcaaccagaatggcatatgcgacggccatggactggttcatagccgtctgttatgtggatagtgaagaaattttctccatccctactccgggtggaacta

SEQ ID NO: 62 - RNA, 360 nt, synthetic construct; identical to SEQ ID NO: 50
  source 1..360: mol_type = other RNA

SEQ ID NO: 63 - RNA, 105 nt, synthetic construct
  source 1..105: mol_type = other RNA
atgagtgtttccttctatccgcgtcattgttggcatatgcgacggccatggactggttcatagccgtctgttatgtgggccttacgtaccgatagaccactggtg

SEQ ID NO: 64 - RNA, 135 nt, synthetic construct; identical to SEQ ID NO: 56
  source 1..135: mol_type = other RNA

SEQ ID NO: 65 - RNA, 147 nt, synthetic construct
  source 1..147: mol_type = other RNA
gtgtggaccaagtcgacacggatgagtgtttccttctatccgcgtcattgttggcatatgcgacggccatggactggttcatagccgtctgttatgtgggccttacgtaccgatagaccactggtgcaaatagagagctatcttagc

SEQ ID NO: 66 - RNA, 225 nt, synthetic construct
  source 1..225: mol_type = other RNA
attaccactcgacaccgaagtatgttccgcggagtacgcgtgtggaccaagtcgacacggatgagtgtttccttctatccgcgtcattgttggcatatgcgacggccatggactggttcatagccgtctgttatgtgggccttacgtaccgatagaccactggtgcaaatagagagctatcttagcattacgcaaccgcgcccacccttaacgatcatgtcgacg

SEQ ID NO: 67 - RNA, 405 nt, synthetic construct
  source 1..405: mol_type = other RNA
ggttcactcgccgacagtcaggcaggtttcttctactttcaaagctgttgctacgtgagtattcctcagactctgccacaaccggcatctattaccactcgacaccgaagtatgttccgcggagtacgcgtgtggaccaagtcgacacggatgagtgtttccttctatccgcgtcattgttggcatatgcgacggccatggactggttcatagccgtctgttatgtgggccttacgtaccgatagaccactggtgcaaatagagagctatcttagcattacgcaaccgcgcccacccttaacgatcatgtcgacgagatcagttacttatttcccgctacacctgctctccgcggagcacttcggcgcgcaaatcctgcctgagggtttggtagacaaggcaggg

SEQ ID NO: 68 - RNA, 143 nt, synthetic construct
  source 1..143: mol_type = other RNA
atgggatccataattcccccaccacctcccatatgtccagattctcttgatgatgctgatgctttgggaagtatgttaatttcatggtacatgagtggctatcatactggctattatatggaaatgctggcatagagcagcac

SEQ ID NO: 69 - RNA, 198 nt, synthetic construct
  source 1..198: mol_type = other RNA
atgggatccataattcccccaccacctcccatatgtccagattctcttgatgatgctgatgctttgggaagtatgttaatttcatggtacatgagtggctatcatactggctattatatgggttttagacaaaatcaaaaagaaggaaggtgctcacattccttaaatataaggagaaatgctggcatagagcagcac

SEQ ID NO: 70 - RNA, 117 nt, synthetic construct
  source 1..117: mol_type = other RNA
agcatttctccttatattgaaggaatgcgagcaccttggcatatgcgacggccatggactggttcatagccgtctgttatgtccttctttttgattttgtctaaaacccatataata

SEQ ID NO: 71 - RNA, 135 nt, synthetic construct
  source 1..135: mol_type = other RNA
ctctatgccagcatttctccttatattgaaggaatgcgagcaccttggcatatgcgacggccatggactggttcatagccgtctgttatgtccttctttttgattttgtctaaaacccatataatagccagtatg

SEQ ID NO: 72 - RNA, 147 nt, synthetic construct
  source 1..147: mol_type = other RNA
gtgctgctctatgccagcatttctccttatattgaaggaatgcgagcaccttggcatatgcgacggccatggactggttcatagccgtctgttatgtccttctttttgattttgtctaaaacccatataatagccagtatgatagcc

SEQ ID NO: 73 - RNA, 36 nt, synthetic construct; identical to SEQ ID NO: 12
  source 1..36: mol_type = other RNA

SEQ ID NO: 74 - RNA, 36 nt, synthetic construct; identical to SEQ ID NO: 12
  source 1..36: mol_type = other RNA

SEQ ID NO: 75 - RNA, 18 nt, synthetic construct
  source 1..18: mol_type = other RNA
agtgataatttgataacg

SEQ ID NO: 76 - RNA, 216 nt, synthetic construct
  source 1..216: mol_type = other RNA
agcatttattgtagcaaagagtgggtagggacaggagctctaggactggccagtgggtgttctagaggccagctggggttggaagacaatatgcgacggccatggactggttcatagccgtctgttggtctggacacttcactgggtggcaggctgtgttccaagttccacgaaatagctcaagaagttaaccagttccgttccagccttcttgat

SEQ ID NO: 77 - RNA, 474 nt, synthetic construct
  source 1..474: mol_type = other RNA
aggcacagacaccaaggacagagacgctggctaggccgccctccccactgttaccaacatgaagctgctcgcagcaactgtgctactcctcaccatctgcagccttgaaggagctttggttcggagacaggcaaaggagccatgtgtggagagcctggtttctcagtacttccagaccgtgactgactatggcaaggacctgatggagaaggtcaagagcccagagcttcaggccgaggccaagtcttactttgaaaagtcaaaggagcagctgacacccctgatcaagaaggctggaacggaactggttaacttcttgagctatttcgtggaacttggaacacagcctgccacccagtgaagtgtccagaccattgtcttccaaccccagctggcctctagaacacccactggccagtcctagagctcctgtccctacccactctttgctacaataaatgctgaatgaatcca

SEQ ID NO: 78 - RNA, 360 nt, synthetic construct
  source 1..360: mol_type = other RNA
ccaggaggcaccataagtgcctgagactgaggtgccccagtaatcgccgggtagggagcccgctggttgagtggtgacctttactcgtcttctcacattatggaagcccttccgggaatggtggacgatacactgtctatctgcagttgcccatctatggccacggatggggatagattggcgcaatagggtccgcgtctcgctaaaagttacccgaggcgttaacattaaactatagccgatagttatcacaggcaagcgggtagttggttgagagatagcgaggcctgccgtccggaaagataagctcccgtgtacaaccaacatcaatagactcgatgagcgcgccaagaatttaag

(19) United States
(12) Patent Application Publication
(10) Pub. No.: US 2026/0250787 A1
(43) Pub. Date: Aug. 27, 2026

(54) Modular RNA-Based RNA Sensors Utilizing ADAR Editing

(71) Applicants: The Board of Trustees of the Leland Stanford Junior University, Stanford, CA (US); Radar Therapeutics Inc., Berkeley, CA (US)

(72) Inventors: Xiaojing Gao, Stanford, CA (US); Kristjan E. Kaseniit, Stanford, CA (US); Natalie S. Kolber, Stanford, CA (US); Jingwei Song, Stanford, CA (US)

(21) Appl. No.: 19/391,116

(22) Filed: Nov. 17, 2025

Related U.S. Application Data
(63) Continuation of application No. PCT/US2024/031501, filed on May 29, 2024.
(60) Provisional application No. 63/469,774, filed on May 30, 2023.

(51) Int. Cl. C12Q 1/6897; C12N 9/78; C12N 15/11; C12N 15/88

(52) U.S. Cl. CPC C12Q 1/6897; C12N 9/78; C12N 15/111; C12N 15/88; C12Y 305/04004

(57) ABSTRACT

The present disclosure provides methods, compositions, systems, and kits for expressing a protein in a target cell.

### CROSS-REFERENCE TO RELATED APPLICATION

This application is a continuation of PCT Application No. PCT/US2024/031501 filed on May 29, 2024, which application, pursuant to 35 U.S.C. § 119(e), claims priority to the filing date of U.S. Provisional Application Ser. No. 63/469,774 filed on May 30, 2023, the disclosures of which applications are herein incorporated by reference.

### GOVERNMENT RIGHTS

This invention was made with Government support under contract 1656518 (FELLOWSHIP) awarded by the National Science Foundation and under contracts EB027723 and EB033858 awarded by the National Institutes of Health. The Government has certain rights in the invention.

### REFERENCE TO AN ELECTRONIC SEQUENCE LISTING

The contents of the electronic sequence listing (STAN-2114WO_Seq_List.xml; Size: 101,189 bytes; and Date of Creation: May 29, 2024) is herein incorporated by reference in its entirety.

### BACKGROUND

Single-cell transcriptomics often serve as the de facto way to define cell types and states, but targeting cells based on their RNA profile has remained challenging. RNA sense-response systems may enable the identification and destruction of harmful cells (e.g., in the contexts of cancer and autoimmune disorders), or the experimental manipulation of specific cells in a complex environment (e.g., the nervous and the immune systems). Available RNA sensing technologies can be limited to miRNAs, or require careful design around functional RNA structures such as ribozymes, guide RNAs or internal ribosome entry sites. For the latter, an additional confounding factor can be the cell's natural response to double-stranded RNA (dsRNA). dsRNA editing by adenosine deaminases acting on RNA (ADARs) allows for the editing of specific RNAs.

### Summary

Provided herein, in some aspects, are methods and kits for expressing proteins in target cells utilizing ADAR editing.

In some aspects, the present disclosure provides for a method for expressing a protein in a target cell, the method comprising: contacting to the target cell a sensor RNA or a vector encoding a sensor RNA, comprising: (i) a first nucleotide sequence comprising: (1) a nucleotide sequence comprising a region that hybridizes to a target RNA; and (2) a stem-loop sequence comprising one or more editable codons, and (ii) a second nucleotide sequence encoding an output protein; wherein: the target RNA is present in the target cell, and (a) the stem-loop sequence, the sensor RNA, or a region between the first and second nucleotide sequences comprises one or more stop codons that are out of frame of the editable codon, or (b) the stem-loop sequence comprises a sequence that is at least 80% identical to a sequence selected from the group consisting of: SEQ ID NO: 10, SEQ ID NO: 11, and SEQ ID NO: 12, or (c) the target RNA comprises one or more base mismatches opposite of the nucleotide sequence comprising the region that hybridizes to the target RNA, or (d) the region that hybridizes to the target RNA comprises one or more mismatch opposite the target RNA 25 or more nucleotides 5′ or 3′ of the editable codon. In some embodiments, the stem-loop sequence, the sensor RNA, or the region between the first and second nucleotide sequences comprises one or more stop codons that are out of frame of the editable codon. In some embodiments, the editable codon is a stop codon, a start codon, or an AUA codon. In some embodiments, the editable codon comprises one or more bases that are mismatched with the stem-loop sequence opposite the one or more editable codons. In some embodiments, the target RNA comprises one or more base mismatches opposite of the nucleotide sequence comprising the region that hybridizes to the target RNA. In some embodiments, the target RNA comprises ten or more base mismatches opposite of the nucleotide sequence comprising the region that hybridizes to the target RNA. In some embodiments, the region that hybridizes to the target RNA comprises one or more base mismatches opposite the target RNA 25 or more nucleotides 5′ or 3′ of the editable codon. In some embodiments, the sensor RNA comprises a region hybridizing to the target RNA 5′ of the stem-loop sequence. In some embodiments, the sensor RNA further comprises a region hybridizing to the target RNA 3′ of the stem-loop sequence. In some embodiments, the sensor RNA further comprises a region hybridizing to the target RNA 5′ to the stem-loop sequence and a region hybridizing to the target RNA 3′ to the stem-loop sequence. In some embodiments, the sensor RNA further comprises a 5′ UTR 5′ to the first nucleotide sequence or a 3′ UTR 3′ of the second nucleotide sequence. In some embodiments, the 5′ UTR or the 3′ UTR are selected from the group consisting of: a Hs Peg10 5′ and 3′ UTR, a mmPeg10 5′ and 3′ UTR, a HsPNMA1 5′ and 3′ UTR, a mmPNMA1 5′ and 3′ UTR, a HsPNMA3 5′ and 3′ UTR, a mmPNMA3 5′ and 3′ UTR, a HsMAOP1 5′ and 3′ UTR, a mmMAOP1 5′ and 3′ UTR, a HsPNMA5 5′ and 3′ UTR, a mmPNMA5 5′ and 3′ UTR, a HsRTL1 5′ and 3′ UTR, a mmRTL1 5′ and 3′ UTR, a HsZCCHC12 5′ and 3′ UTR, a mmZCCHC12 5′ and 3′ UTR, a HsASPRV1 5′ and 3′ UTR, a mmADPRV1 5′ and 3′ UTR, a HsARC1 5′ and 3′ UTR, and a mmARC1 5′ and 3′ UTR. In some embodiments, the stem-loop sequence comprises a sequence that is at least 80% identical to a sequence selected from the group consisting of: SEQ ID NO: 10, SEQ ID NO: 11, and SEQ ID NO: 12. In some embodiments, the sensor RNA comprises a cleavage domain or a 2A self-cleaving domain between the first nucleotide sequence and the second nucleotide sequence. In some embodiments, the output protein is selected from a fluorescent protein, a genomic modification protein, a transcription factor, a killing factor, a toxin, an antigen, a T cell receptor, a chimeric antigen receptor, a therapeutic protein, and an enzyme. In some embodiments, the target RNA is associated with a disease, condition, cell type, or tissue. In some embodiments, the sensor RNA comprises one or more pseudouridines or the sensor nucleotide sequence does not comprise pseudouridines. In some embodiments, the contacting to the target cell comprises contacting the target cell with an adeno-associated virus (AAV) comprising the sensor RNA wherein the sensor RNA is encoded in an AAV vector. In some embodiments, contacting comprises administering to a patient. In some embodiments, the sensor RNA further comprises: (i) a fourth nucleotide sequence comprising a second cleavage domain wherein the fourth nucleotide sequence precedes the first nucleotide sequence and (ii) a fifth nucleotide sequence comprising a nucleotide sequence encoding a marker protein wherein the fifth nucleotide sequence precedes the fourth nucleotide sequence. In some embodiments, the method further comprises contacting the target cell with an adenosine deaminase acting on RNA (ADAR) protein or a coding sequence thereof. In some embodiments, the method further comprises assaying for the presence of the output protein. In some embodiments, the assaying comprises using microscopy, flow cytometry, immunoblotting, a plate reader, or a combination thereof. In some embodiments, the target RNA comprises a cellular mRNA. In some embodiments, the region that hybridizes to the target RNA comprises a 5′ or 3′ UTR of the cellular mRNA.

In some aspects, the present disclosure provides for a sensor RNA or a vector encoding a sensor RNA for expressing a protein in a target cell, comprising: (i) a first nucleotide sequence comprising: (1) a nucleotide sequence comprising a region that hybridizes to a target RNA; and (2) a stem-loop sequence comprising one or more editable codons, and (ii) a second nucleotide sequence encoding an output protein; wherein: the target RNA is present in the target cell, and (a) the stem-loop sequence, the sensor RNA, or a region between the first and second nucleotide sequences comprises one or more stop codons that are out of frame of the editable codon, or (b) the stem-loop sequence comprises a sequence that is at least 80% identical to a sequence selected from the group consisting of: SEQ ID NO: 10, SEQ ID NO: 11, and SEQ ID NO: 12, or (c) the target RNA comprises one or more base mismatches opposite of the nucleotide sequence comprising the region that hybridizes to the target RNA, or (d) the region that hybridizes to the target RNA comprises one or more mismatch opposite the target RNA 25 or more nucleotides 5′ or 3′ of the editable codon. In some embodiments, the stem-loop sequence, the sensor RNA, or the region between the first and second nucleotide sequences comprises one or more stop codons that are out of frame of the editable codon. In some embodiments, the editable codon is a stop codon, a start codon, or an AUA codon. In some embodiments, the editable codon comprises one or more bases that are mismatched with the stem-loop sequence opposite the one or more editable codons. In some embodiments, the target RNA comprises one or more base mismatches opposite of the nucleotide sequence comprising the region that hybridizes to the target RNA. In some embodiments, the target RNA comprises ten or more base mismatches opposite of the nucleotide sequence comprising the region that hybridizes to the target RNA. In some embodiments, the region that hybridizes to the target RNA comprises one or more base mismatches opposite the target RNA 25 or more nucleotides 5′ or 3′ of the editable codon. In some embodiments, the sensor RNA comprises a region hybridizing to the target RNA 5′ of the stem-loop sequence. In some embodiments, the sensor RNA further comprises a region hybridizing to the target RNA 3′ of the stem-loop sequence. In some embodiments, the sensor RNA further comprises a region hybridizing to the target RNA 5′ to the stem-loop sequence and a region hybridizing to the target RNA 3′ to the stem-loop sequence. In some embodiments, the sensor RNA further comprises a 5′ UTR 5′ to the first nucleotide sequence or a 3′ UTR 3′ of the second nucleotide sequence. In some embodiments, the 5′ UTR or the 3′ UTR are selected from the group consisting of: a Hs Peg10 5′ and 3′ UTR, a mmPeg10 5′ and 3′ UTR, a HsPNMA1 5′ and 3′ UTR, a mmPNMA1 5′ and 3′ UTR, a HsPNMA3 5′ and 3′ UTR, a mmPNMA3 5′ and 3′ UTR, a HsMAOP1 5′ and 3′ UTR, a mmMAOP1 5′ and 3′ UTR, a HsPNMA5 5′ and 3′ UTR, a mmPNMA5 5′ and 3′ UTR, a HsRTL1 5′ and 3′ UTR, a mmRTL1 5′ and 3′ UTR, a HsZCCHC12 5′ and 3′ UTR, a mmZCCHC12 5′ and 3′ UTR, a HsASPRV1 5′ and 3′ UTR, a mmADPRV1 5′ and 3′ UTR, a HsARC1 5′ and 3′ UTR, and a mmARC1 5′ and 3′ UTR. In some embodiments, the stem-loop sequence comprises a sequence that is at least 80% identical to a sequence selected from the group consisting of: SEQ ID NO: 10, SEQ ID NO: 11, and SEQ ID NO: 12. In some embodiments, the sensor RNA comprises a cleavage domain or a 2A self-cleaving domain between the first nucleotide sequence and the second nucleotide sequence. In some embodiments, the output protein is selected from a fluorescent protein, a genomic modification protein, a transcription factor, a killing factor, a toxin, an antigen, a T cell receptor, a chimeric antigen receptor, a therapeutic protein, and an enzyme. In some embodiments, the target RNA is associated with a disease, condition, cell type, or tissue. In some embodiments, the sensor RNA comprises one or more pseudouridines or the sensor nucleotide sequence does not comprise pseudouridines. In some embodiments, the sensor RNA further comprises: (i) a fourth nucleotide sequence comprising a second cleavage domain wherein the fourth nucleotide sequence precedes the first nucleotide sequence and (ii) a fifth nucleotide sequence comprising a nucleotide sequence encoding a marker protein wherein the fifth nucleotide sequence precedes the fourth nucleotide sequence. In some embodiments, the target RNA comprises a cellular mRNA. In some embodiments, the region that hybridizes to the target RNA comprises a 5′ or 3′ UTR of the cellular mRNA.

In some aspects, the current disclosure provides for a host cell comprising any of the RNAs or sensor RNAs described herein.

In some aspects, the present disclosure provides for a sensor RNA or a vector encoding a sensor RNA as described herein.

In some aspects, the present disclosure provides for an LNP comprising a sensor RNA according to any of the aspects or embodiments described herein.

In some aspects, the present disclosure provides for a pharmaceutically acceptable composition comprising any of the sensor RNAs, vectors or LNPs described herein and a pharmaceutically acceptable carrier.

In some aspects, the present disclosure provide a method for expressing a protein in a target cell, the method containing: combining the target cell with a sensor RNA containing: (i) a first nucleotide sequence comprising a sensor nucleotide sequence that hybridizes to the target RNA wherein the sensor nucleotide sequence comprises a stem-loop sequence comprising one or more editable codons, (ii) a second nucleotide sequence encoding a first cleavage domain, and (iii) a third nucleotide sequence encoding an output protein; wherein: the target RNA is present in the target cell, and a) the stem-loop sequence or the second nucleotide sequence comprises one or more stop codons that are out of frame of the editable codon, b) the stem-loop sequence comprises a sequence that is at least 80% identical, at least at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or substantially identical from a sequence identity standpoint to a sequence selected from the group consisting of: SEQ ID NO: 10, SEQ ID NO: 11, and SEQ ID NO: 12, c) the target RNA comprises one or more base mismatches opposite of the stem-loop sequence, or d) the sensor nucleotide sequence comprises one or more mismatch 25 or more base pairs upstream (e.g 5′) or downstream (e.g. 3′) of the editable codon.

The present disclosure provides a method for expressing a protein in a target cell, the method containing: combining the target cell with a sensor RNA comprising: (i) a first nucleotide sequence containing a sensor nucleotide sequence that hybridizes to the target RNA, wherein the sensor nucleotide sequence containing a stem-loop sequence containing one or more editable codons, (ii) a second nucleotide sequence encoding a first cleavage domain, and (iii) a third nucleotide sequence encoding an output protein; wherein: the target RNA is present in the target cell, and the stem-loop sequence is defined by a sequence selected from the group consisting of SEQ ID NO:10, SEQ ID NO: 11, and SEQ ID NO: 12.

In some aspects, the present disclosure also provides a method for expressing a protein in a target cell, the method containing: combining the target cell with a sensor RNA containing: (i) a first nucleotide sequence containing a sensor nucleotide sequence that hybridizes to the target RNA, wherein the sensor nucleotide sequence contains a stem-loop sequence comprising one or more editable codons, (ii) a second nucleotide sequence encoding a first cleavage domain, and (iii) a third nucleotide sequence encoding an output protein; wherein: the target RNA is present in the target cell, and the target RNA contains one or more base mismatch opposite of the stem-loop sequence.

In some aspects, the present disclosure also provides a method for expressing a protein in a target cell, the method containing: combining the target cell with a sensor RNA containing: (i) a first nucleotide sequence comprising a sensor nucleotide sequence that hybridizes to the target RNA wherein the sensor nucleotide sequence comprises a stem-loop sequence containing one or more editable codons, (ii) a second nucleotide sequence encoding a first cleavage domain, and (iii) a third nucleotide sequence encoding an output protein; wherein: the target RNA is present in the target cell, and the sensor nucleotide sequence contains one or more mismatch 25 or more base pairs upstream or downstream (e.g. 3′) of the editable codon.

In some aspects, the present disclosure also provides a method for expressing a protein in a target cell, the method containing: combining the target cell with a sensor RNA containing: (i) a first nucleotide sequence containing a sensor nucleotide sequence that hybridizes to the target RNA wherein the sensor nucleotide sequence contains a stem-loop sequence containing one or more editable codons, (ii) a second nucleotide sequence encoding a first cleavage domain, and (iii) a third nucleotide sequence encoding an output protein; wherein: the target RNA is present in the target cell, and the stem-loop sequence contains one or more stop codons that are out of frame of the editable codon.

In some aspects, the present disclosure also provides a method for generating a pseudouridine-containing sensor RNA, the method containing: combining: (i) a first segment containing: (ia) a first nucleotide sequence containing a nucleotide sequence encoding a marker protein, and (ib) a second nucleotide sequence containing a first cleavage domain, wherein the first segment contains one or more pseudouridines; and (ii) a second segment containing: a third nucleotide sequence containing a sensor nucleotide sequence that hybridizes to the target RNA, wherein the sensor nucleotide sequence contains a stem-loop sequence containing one or more editable codons, wherein the second segment does not contain a pseudouridine; (iii) a third segment containing: (iiia) a fourth nucleotide sequence encoding a first cleavage domain, and (iiib) a fifth nucleotide sequence encoding an output protein, wherein the third segment contains one or more pseudouridines.

Kits for practicing the subject methods are also provided, in some aspects of the disclosure.

### BRIEF DESCRIPTION OF THE FIGURES

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1C disclose the effects of different stem-loop sequences or the length of the nucleotide sequence that hybridizes to the target or predetermined RNA (“sensor”) on the production of the output protein.

FIG. 2 depicts detection of alternatively spliced variants using sensor RNAs with varying lengths of nucleotide sequences that bind to the target or predetermined RNA (“sensor”).

FIG. 3 depicts a schematic of an example ModulADAR.

FIG. 4 depicts an example schematic of methods for generating a pseudouridine-containing sensor RNA.

FIG. 5 depicts graphs of luminescence versus each transfection condition for the experiment described in Example 2. In this experiment either SP047, SP019, or SP127 vectors were co-transfected with a trigger sequence (“trigger”) or a negative control sequence (“neg trigger”) into either HEK293 cells (“HEKwt”), 293FT cells (“293FT”), and HEK293-JumpIn cells (“Jumpin”). In each cell line measured, the non-specific activation of the sensor (“negative trigger”) was decreased between the SP019 and SP127 conditions, indicating that the introduction of the out of frame stop codon in SP127 reduced non-specific activation of the sensor.

FIGS. 6A, 6B, 6C, and 6D depict graphs of editing efficiency for the pooled RNA sensor experiments described in Example 3. FIGS. 6A and 6B show the results where 3 nucleotide long tiled mismatches were introduced throughout the length of the upstream (e.g. 5′) and downstream (e.g. 3′) trigger-hybridizing or non-targeting nucleotide sequences flanking the stem-loops, with FIG. 6A representing the results for mismatches introduced in the upstream trigger-hybridizing and FIG. 6B representing the results for mismatches introduced in the downstream trigger-hybridizing. FIGS. 6C and 6D show the results where 3 nucleotide long tiled inserts (“bulges”) were introduced throughout the length of the upstream (e.g. 5′) and downstream (e.g. 3′) trigger-hybridizing or non-targeting nucleotide sequences flanking the stem-loops, with FIG. 6C representing the results for inserts introduced in the upstream trigger-hybridizing nucleotide sequences and FIG. 6D representing the results for inserts introduced in the downstream trigger-hybridizing nucleotide sequences. For each of FIGS. 6A, 6B, 6C, and 6D, editing efficiency of the editable codon within the sensor construct is shown on the y-axis as fraction edited out of all sequences detected, wherein the x-axis indicates distance in nucleotides of the mismatch or insert upstream or downstream from the edited A of the editable codon of the sensor RNA. Also for each of FIGS. 6A, 6B, 6C, and 6D, editing efficiency of the editable codon for the SP478-derived RNA sensor sequence is shown in the circular data points when paired with the matching trigger (“APOA2 trigger”), whereas editing efficiency of the editable codon when co-transfected with a control is shown in the triangular data points (“mismatching trigger”). FIGS. 6A, 6B, 6C, and 6D show that mismatches and inserts are tolerated throughout the length of both the upstream and downstream sequences flanking the stem-loops of the sensor (as editing is not abrogated at any of the data points), and that in some instances (see e.g. FIG. 6C, which shows that inserts at about 40 or 42 nucleotides upstream of the stem-loop improve editing efficiency), the mismatches or inserts improve efficiency of editing at the editable codon.

### DETAILED DESCRIPTION

### Summary

Before describing exemplary embodiments in greater detail, the following definitions are set forth to illustrate and define the meaning and scope of the terms used in the description.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

In general, terms used herein are generally intended as “open” terms (e.g., the term “including” is to be interpreted as “including but not limited to,” the term “having” is to be interpreted as “having at least,” the term “includes” is to be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases need not be construed to imply that the introduction of a claim recitation by the indefinite articles “a” or “an” limits any particular claim containing such introduced claim recitation to embodiments containing solely one such recitation, even when the same claim includes the introductory phrases “one or more” or “at least one” and indefinite articles such as “a” or “an” (e.g., “a” or “an” is to be interpreted to mean “at least one” or “one or more”); the same holds true for the use of definite articles used to introduce claim recitations. In addition, even if a specific number of an introduced claim recitation is explicitly recited, those skilled in the art will recognize that such recitation is to be interpreted to mean at least the recited number (e.g., the bare recitation of “two recitations,” without other modifiers, generally signifies at least two recitations, or two or more recitations). Furthermore, in those instances where a convention analogous to “at least one of A, B, and C, etc.” is used, in general such a construction is intended in the sense one having skill in the art may understand the convention (e.g., “a system having at least one of A, B, and C” may include but not be limited to systems that have A alone, B alone, C alone, A and B together, A and C together, B and C together, or A, B, and C together, etc.). In those instances where a convention analogous to “at least one of A, B, or C, etc.” is used, in general such a construction is intended in the sense one having skill in the art may understand the convention (e.g., “a system having at least one of A, B, or C” may include but not be limited to systems that have A alone, B alone, C alone, A and B together, A and C together, B and C together, or A, B, and C together, etc.).

In general, as used herein, virtually any disjunctive word and/or phrase presenting two or more alternative terms, is to be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups herein, the disclosure is also generally intended to encompass any individual member or subgroup of members of the Markush group.

In general, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, a range generally includes each individual member. Thus, for example, a group having 1-3 articles refers to groups having 1, 2, or 3 articles. Similarly, a group having 1-5 articles refers to groups having 1, 2, 3, 4, or 5 articles, and so forth.

As used herein, the singular forms “a”, “an”, and “the” generally include plural referents unless the context clearly dictates otherwise. For example, the term “a RNA sensor” refers to one or more RNA sensors, e.g., a single RNA sensor and multiple RNA sensors. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Reference to an item in the singular is to be understood as including the plural and vice versa unless explicitly stated otherwise or clear from the text. Grammatical conjunctions are intended to express any and all disjunctive conjunctions and conjunctions of conjunctive clauses, sentences, words, and the like, unless otherwise stated or clear from the context. Thus, the term “or” is generally understood to be an inclusive “or” encompassing both alternatives.

The terms “polynucleotide” and “nucleic acid,” used interchangeably herein, generally refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. The terms “polynucleotide” and “nucleic acid” generally are understood to include, as applicable to the embodiment being described, single-stranded (such as sense or antisense) and double-stranded polynucleotides.

By “hybridizable” or “complementary” or “substantially complementary” it is generally meant that a nucleic acid (e.g. RNA, DNA) contains a sequence of nucleotides that enables it to non-covalently bind, e.g. form Watson-Crick base pairs or G/U base pairs, “anneal”, or “hybridize,” to another nucleic acid in a sequence-specific, antiparallel, manner (e.g., a nucleic acid specifically binds to a complementary nucleic acid) under the appropriate in vitro or in vivo conditions of temperature and solution ionic strength. Standard Watson-Crick base-pairing includes: adenine/adenosine) (A) pairing with thymidine/thymidine (T), A pairing with uracil/uridine (U), and guanine/guanosine) (G) pairing with cytosine/cytidine (C). Inosine (I) bases pair with cytosine/cytidine. In addition, for hybridization between two RNA molecules (e.g., dsRNA), and for hybridization of a DNA molecule with an RNA molecule (e.g., when a DNA splint oligo base pairs with an mRNA segment, etc.): G can also base pair with U. For example, G/U base-pairing is partially responsible for the degeneracy (e.g., redundancy) of the genetic code in the context of tRNA anti-codon base-pairing with codons in mRNA. Thus, in the context of this disclosure, a G (e.g., of a protein-binding segment (e.g., dsRNA duplex) of a guide RNA molecule; of a target nucleic acid (e.g., target DNA or RNA) base pairing with a sensor RNA) is considered complementary to both a U and to C. For example, when a G/U base-pair can be made at a given nucleotide position of a protein-binding segment (e.g., dsRNA duplex) of a sensor RNA molecule, the position is not considered to be non-complementary, but is instead considered to be complementary. Pseudouridine (pseudo-U, Ψ) is meant to stand for an isomer of uridine, where the uracil nucleobase is attached through a carbon-carbon linkage to the sugar. The pseudouridine is in some cases modified. In some cases the pseudouridine modification is methylation, e.g., at the N1 position, forming N1-methylpseudouridine. Pseudouridine and its modifications base pair like uridine. When U is shown in a sequence, e.g. UAG, the U may be a pseudouridine. When a T is shown in a sequence, it is meant that the RNA encoded by that sequences contains a U, a pseudouridine, or a modified pseudouridine. The DNA that encodes a U or pseudouridine contains T in place of U or pseudouridine. “Mismatched” generally signifies refers to a base that is opposite a non-complementary base in an otherwise double-stranded structure (e.g., a C:A mismatch), or that a base is opposite no bases (e.g., a base is in a loop structure).

Hybridization generally requires that the two nucleic acids contain complementary sequences, although mismatches between bases are possible. The conditions appropriate for hybridization between two nucleic acids depend on the length of the nucleic acids and the degree of complementarity. The greater the degree of complementarity between two nucleotide sequences, the greater the value of the melting temperature (Tm) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more).

It is generally understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure, a ‘bulge’, and the like). A polynucleotide can include 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region (e.g. is capable of specific hybridization) would represent 90 percent complementarity. The remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “peptide,” “polypeptide,” and “protein” are generally used interchangeably herein, and refer to a polymeric form of amino acids of any length, which can include coded and non-coded amino acids, chemically or biochemically modified or derivatized amino acids, and polypeptides having modified peptide backbones.

The term “naturally-occurring” as used herein as applied to a nucleic acid, a protein, a cell, or an organism, generally refers to a nucleic acid, protein, cell, or organism that is found in nature. For example, a polypeptide or polynucleotide sequence that is present in an organism (including viruses) that can be isolated from a source in nature and which has not been intentionally modified by a human in the laboratory is naturally occurring.

The term “exogenous” as used herein as applied to a nucleic acid or a protein generally refers to a nucleic acid or protein that is not normally or naturally found in or produced by a given bacterium, organism, or cell in nature. As used herein, the term “endogenous nucleic acid” refers to a nucleic acid that is normally found in or produced by a given bacterium, organism, or cell in nature. An “endogenous nucleic acid” is also referred to as a “native nucleic acid” or a nucleic acid that is “native” to a given bacterium, organism, or cell. As used herein, the term “endogenous polypeptide” refers to a polypeptide that is normally found in or produced by a given bacterium, organism, or cell in nature.

“Recombinant,” as used herein, generally signifies that a particular nucleic acid or protein is the product of various combinations of cloning, restriction, or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA containing the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a predetermined product by various mechanisms.

Thus, e.g., the term “recombinant” nucleic acid or “recombinant” protein generally refers to one which is not naturally occurring, e.g., is made by the artificial combination of two otherwise separated segments of sequence through human intervention. This artificial combination is often accomplished by either chemical synthesis methods, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of predetermined functions to generate a predetermined combination of functions. This artificial combination is often accomplished by either chemical synthesis methods, or by the artificial manipulation of isolated segments of nucleic acids, e.g., by genetic engineering techniques.

“Construct” or “vector” generally refers to a recombinant nucleic acid, generally recombinant DNA, which has been generated for the purpose of the expression or propagation of a nucleotide sequence(s) of interest, or is to be used in the construction of other recombinant nucleotide sequences.

In some examples, a vector is a minicircle, plasmid, nanoplasmid, yeast artificial chromosome (YAC), bacterial artificial chromosome (BAC), cosmid, phagemid, bacteriophage genome, or baculovirus genome. Suitable vectors also include vectors derived from bacteriophages or plant, invertebrate, or animal (including human) viruses such as CELiD vectors, doggybone DNA (dbDNA) vectors, closed-end linear duplex DNA vectors (e.g. wherein each end is covalently closed by chemical modification), adeno-associated viral vectors (e.g. AAV1, AAV2, AAV4, AAV5, AAV6, AAV7, AAV8, AAV9, or pseudotyped combinations thereof such as AAV2/5, AAV2/2, AAV-DJ, or AAV-DJ8), retroviral vectors (e.g. MLV or self-inactivating or SIN versions thereof, or pseudotyped versions thereof), herpesviral (e.g. HSV- or EBV-based), lentiviral vectors (e.g. HIV-, FIV-, or EIAV-based, or pseudotyped versions thereof), or adenoviral vectors (e.g. Ad5-based, including replication-deficient, replication-competent, or helper-dependent versions thereof).

The term “lipid” generally refers to a group of organic compounds that include, but are not limited to, esters of fatty acids and are characterized by being insoluble in water, but soluble in many organic solvents. They can be divided into at least three classes: (1) “simple lipids,” which can include fats and oils as well as waxes; (2) “compound lipids,” which can include phospholipids and glycolipids; and (3) “derived lipids” which can include steroids.

The term “lipid particle” generally includes a lipid formulation that can be used to deliver an active agent or therapeutic agent, such as a nucleic acid (e.g., an RNA as described herein or a vector encoding the RNA), to a target or predetermined site of interest (e.g., cell, tissue, organ, and the like). In some embodiments, the lipid particle of the invention is a lipid nanoparticle, which can be formed from a cationic lipid, a non-cationic lipid, and optionally a conjugated lipid that prevents aggregation of the particle. In other embodiments, the active agent or therapeutic agent, such as a nucleic acid, may be encapsulated in the lipid portion of the particle, thereby protecting it from enzymatic degradation.

As used herein, the term “LNP” generally refers to a lipid nanoparticle. An LNP generally represents a particle made from lipids (e.g., a cationic lipid, a non-cationic lipid, and optionally a conjugated lipid that prevents aggregation of the particle), wherein the nucleic acid (e.g., an RNA as described herein, or a or vector encoding the RNA) can be fully encapsulated within the lipid. In certain instances, LNP can be useful for systemic applications, as they can exhibit extended circulation lifetimes following intravenous (i.v.) injection, they can accumulate at distal sites (e.g., sites physically separated from the administration site), and they can mediate silencing of target gene expression at these distal sites. The nucleic acid may be complexed with a condensing agent and encapsulated within an LNP (see e.g. PCT Publication No. WO 00/03683, the disclosure of which is herein incorporated by reference in its entirety for all purposes). As described herein, any of the RNAs or vectors encoding the RNAs can be encapsulated within an LNP.

As used herein, the term “IVT RNA” generally refers to a nucleic acid molecule encoding a polypeptide sequence to be expressed in a host that can be generated by in vitro transcription and is translatable in a mammalian (and preferably human) cell or subject to produce the polypeptide. Generating the IVT RNA can be accomplished by any suitable technique (e.g. in vitro transcription in cell lysates from vectors encoding the IVT RNA) The transcribed IVT RNA molecule can be modified further post-transcription, e.g., by adding a cap or other functional group. IVT RNAs can comprise a modified ribonucleic acid to reduce immunogenicity (e.g. in place of some or all of a particular canonical nucleotide, such as uracil). Any of the RNAs described herein (e.g. sensor RNAs) can be IVT RNAs.

The term “transformation” or “transfection” generally refers to a permanent or transient genetic change induced in a cell following introduction of a nucleic acid (e.g., DNA or RNA exogenous to the cell). Genetic change (“modification”) can be accomplished either by incorporation of the new DNA into the genome of the host cell, or by transient or stable maintenance of the new DNA as an episomal element. Where the cell is a eukaryotic cell, a permanent genetic change is generally achieved by introduction of the DNA into the genome of the cell. Suitable methods of genetic modification include viral infection, transfection, conjugation, protoplast fusion, electroporation, particle gun technology, calcium phosphate precipitation, direct microinjection, and the like. The choice of method is generally dependent on the type of cell being transformed and the circumstances under which the transformation is taking place (e.g., in vitro, ex vivo, or in vivo). A general discussion of these methods can be found in Ausubel et al, Short Protocols in Molecular Biology, 3rd ed., Wiley & Sons, 1995.

The terms “regulatory region” and “regulatory elements”, generally used interchangeably herein, generally refer to transcriptional and translational control sequences, such as promoters, enhancers, polyadenylation signals, terminators, protein degradation signals, translational start and stop codons, translation initiation sites, splice enhancer/donor/branch/acceptor sites, and the like, that provide for or regulate expression of a coding sequence or production of an encoded polypeptide in a host cell. As used herein, a “promoter sequence” or “promoter” is a DNA regulatory region capable of binding/recruiting RNA polymerase (e.g., via a transcription initiation complex) and initiating transcription of a downstream (3′ direction) sequence (e.g., a protein coding (“coding”) or non-protein-coding (“non-coding”) sequence. A promoter can be a constitutively active promoter (e.g., a promoter that is constitutively in an active/“ON” state), it may be an inducible promoter (e.g., a promoter whose state, active/“ON” or inactive/“OFF”, is controlled by an external stimulus, e.g., the presence of a particular temperature, compound, or protein), it may be a spatially restricted promoter (e.g., tissue specific promoter, cell type specific promoter, etc.), or it may be a temporally restricted promoter (e.g., the promoter is in the “ON” state or “OFF” state during specific stages of embryonic development or during specific stages of a biological process, e.g., hair follicle cycle in mice).

“Operably linked” generally refers to a juxtaposition wherein the components so described are in a relationship permitting them to function in their intended manner. For instance, a promoter is operably linked to a nucleotide sequence (e.g., a protein coding sequence, e.g., a sequence encoding an mRNA; a non-protein coding sequence, e.g., a sequence encoding a Shh protein; and the like) if the promoter affects its transcription or expression.

The term “adenosine deaminase acting on RNA” or “ADAR” generally refers to an enzyme that catalyze the hydrolytic C6 deamination of adenosine (A) to produce inosine (I) in RNA substrates that are double stranded. ADARs preferentially edit double stranded RNAs at sites of mismatches where mismatches containing adenosines and cytosines are editing more efficiently than other mismatches. Editing by ADARs results in nucleotide substitution in RNA, because the purine I generated as the result of the deamination reaction is recognized as G instead of A, both by ribosomes during translational decoding of mRNA and by RNA-dependent polymerases during RNA replication. The term “ADAR” encompasses any documented type of ADAR such as ADAR1 (ADAR) or ADAR2 (ADARB2).

As used herein “ADAR1” generally refers to an adenosine deaminase acting on RNA that catalyzes the hydrolytic C6 deamination of adenosine (A) to produce inosine (I) in RNA substrates that are double stranded. ADAR1 has 2 main isoforms, p150 and p110. The term “ADAR1” encompasses ADAR1 from various species. Amino acid sequences of ADAR1 from various species are publicly available. See, e.g., GenBank Accession Nos. NP_001102 (Homo sapiens ADAR1 p150), NP_001180424.1 (Homo sapiens ADAR1 p110), NP_001139768 (Mus musculus ADAR1 p150), NP_001033676 (Mus musculus ADAR1 p110). The term “ADAR1” as used herein also encompasses fragments, fusion proteins, and variants (e.g., variants having one or more amino acid substitutions, addition, deletions, or insertions) that retain ADAR1 enzymatic activity.

As used herein “ADAR2” generally refers to an adenosine deaminase acting on RNA that catalyzes the hydrolytic C6 deamination of adenosine (A) to produce inosine (I) in RNA substrates that are double stranded. ADAR2 is exclusively localized to the nucleus. The term “ADAR2” encompasses ADAR2 from various species. Amino acid sequences of ADAR2 from various species are publicly available. See, e.g., GenBank Accession Nos. NP_056648.1 (Homo sapiens ADAR2), NP_001020008.1 (Mus musculus ADAR2), AC052474.1 (Doryteuthis opalescens ADAR2). The term “ADAR2” as used herein also encompasses fragments, fusion proteins, and variants (e.g., variants having one or more amino acid substitutions, addition, deletions, or insertions) that retain ADAR2 enzymatic activity.

The term “sample” as used herein generally relates to a material or mixture of materials, typically, although not necessarily, in fluid, e.g., aqueous, form, containing one or more components of interest. Samples may be derived from a variety of sources such as from food stuffs, environmental materials, a biological sample or solid, such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components). In certain embodiments of the method, the sample includes a cell. In some instances of the method, the cell is in vitro. In some instances of the method, the cell is in vivo.

The term “biological sample” generally encompasses a clinical sample or a non-clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's sample cell, e.g., a sample containing polynucleotides or polypeptides that is obtained from a patient's sample cell (e.g., a cell lysate or other cell extract containing polynucleotides or polypeptides); and a sample containing sample cells from a patient. A biological sample containing a sample cell from a patient can also include normal, non-diseased cells. A biological sample may be from a plant or an animal. The biological sample may also be from any species. In certain embodiments of the method, the biological sample includes a cell. In some instances of the method, the cell is in vitro. In some instances of the method, the cell is in vivo.

The term “editable codon” as used herein generally refers to a 3-nucleotide sequence that is editable by an ADAR protein or a derivative thereof. The codon may be a start codon, a stop codon or an AUA codon. The codon contains a sequence that contains an adenosine base. In general, in the methods disclosed herein, the editable codon is a start codon that is edited to become a non-start codon, a stop codon that is edited to become a non-stop codon, or a non-start codon (e.g., AUA) that is edited to become a start codon.

The term “pharmaceutically acceptable carrier” is generally intended to denote any material, which is inert in the sense that it substantially does not have a therapeutic and/or prophylactic effect per se. Such an excipient is added with the purpose of making it possible to obtain a pharmaceutical composition having acceptable technical properties. Examples of suitable pharmaceutically acceptable carriers or diluents include, but are not limited to, ethanol, water, glycerol, propylene glycol, glycerin, diethylene glycol monoethylether, vitamin A and E oils, mineral oil, PPG2 myristyl propionate, magnesium carbonate, potassium phosphate, silicon dioxide, vegetable oils such as castor oil and derivatives thereof, plant gums, gelatin, animal oils, solketal, calcium. carbonate, dibasic calcium phosphate, tribasic calcium phosphate, calcium sulfate, microcrystalline cellulose, powdered cellulose, dextrans, dextrin, dextrose, fructose, kaolin, lactose, mannitol, sorbitol, starch, pre-gelatinized starch, sucrose, sugar etc.

### EXAMPLE EMBODIMENTS

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

The citation of any publication is for its disclosure prior to the filing date and should not be construed as an admission that the present claims are not entitled to antedate such publication by virtue of prior invention. Further, the dates of publication provided can be different from the actual publication dates which can be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

All patents and publications, including all sequences disclosed within such patents and publications, referred to herein are expressly incorporated by reference.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention or disclosure. The upper and lower limits of these smaller ranges may independently be included in the smaller ranges, and are also encompassed within the invention or disclosure, subject to any specifically excluded limit in the stated range. Where the stated range includes one or both of the limits, ranges excluding either or both of those included limits are also included in the invention or disclosure.

It is appreciated that certain features of the invention or disclosure, which are, for clarity, described in the context of separate embodiments, may also be provided in combination in a single embodiment. Conversely, various features of the invention or disclosure, which are, for brevity, described in the context of a single embodiment, may also be provided separately or in any suitable sub-combination. All combinations of the embodiments pertaining to the invention or disclosure are specifically embraced by the present invention or disclosure and are disclosed herein just as if each and every combination was individually and explicitly disclosed. In addition, all sub-combinations of the various embodiments and elements thereof are also specifically embraced by the present invention or disclosure and are disclosed herein just as if each and every such sub-combination was individually and explicitly disclosed herein.

In further describing the subject disclosure, methods for expressing a target protein of interest are described. Next, methods for generating a pseudouridine-containing sensor RNA are described. Furthermore, methods for treating a disease or condition are described.

As summarized above, methods are provided for expressing a protein in a target cell, the methods include combining a cell with a sensor RNA as described above, wherein the target RNA is present in the target cell.

The target RNA may be any RNA. For example, the target RNA includes, without limitation, mRNA, long non-coding RNA, transfer RNA, ribosomal RNA, small RNAs such as microRNA, small interfering RNA, small nucleolar RNAs, etc. In some embodiments, the target RNA may be differentially expressed in different tissues cell types, or cell states and the detecting of the target RNA may be used to identify tissue types, cell types or cell states. In some embodiments, the target RNA may be a genetic variant of a gene. In these instances, the genetic variant may be predictive of a disease or susceptible to a disease such as an oncogenic mutation or a genetic variant associated with increased susceptibility to a pathogen. In some embodiments, the methods of the present disclosure may be used to detect point mutations that are associated with the development of a disease such as cancer, neurodegenerative disease, an autoimmune disease, etc. In some embodiments, the methods of the present disclosure are capable of detecting small indels, single nucleotide polymorphisms (SNPs) or variant, multi-nucleotide variant or dinucleotide variant, etc. In some embodiments, the methods of the present disclosure are capable of detecting and distinguishing copy number variants within and between biological samples. The target RNA may also be a gene fusion which may be predictive of cancer in general or a specific type of cancer. The target RNA may also be a specific splice variant (isoform) of a gene.

The target RNA to which the sensor RNA hybridizes is, in some instances, determined by the target cell. In some embodiments, the target cell is a cell that is in a particular disease state. In these instances, the target cell includes a target RNA that is specific to the disease state or is in a higher abundance in cells that are in a particular disease state such as a cancerous cell. The cell may be in any disease state. In some embodiments, the target cell is a particular cell type. In these instances, the target cell includes a target RNA that is specific to the cell type or is in a higher abundance in cells that are a particular cell type. The cell may be any cell type.

Cells of any origin are candidate cells for combining with a sensor RNA of the present disclosure. Non-limiting examples of candidate cell types include connective tissue elements such as fibroblast, skeletal tissue (bone and cartilage), skeletal, cardiac and smooth muscle, epithelial tissues (e.g., liver, lung, breast, skin, bladder and kidney), neural cells (glia and neurons), endocrine cells (adrenal, pituitary, pancreatic islet cells), bone marrow cells, melanocytes, and many different types of hematopoietic cells. Suitable cells can also be cells representative of a specific body tissue from a subject. The types of body tissues include, but are not limited, to blood, muscle, nerve, brain, heart, lung, liver, pancreas, spleen, thymus, esophagus, stomach, intestine, kidney, testis, ovary, hair, skin, bone, breast, uterus, bladder, spinal cord and various kinds of body fluids.

Cells suitable for use in a subject method include cells of a variety of subject hosts. Generally, such subject hosts are “mammals” or “mammalian”, where these terms are used broadly to describe organisms which are within the class mammalia, including the orders carnivore (e.g., dogs and cats), rodentia (e.g., mice, guinea pigs and rats), and primates (e.g., humans, chimpanzees and monkeys). In many aspects, the subject host will be a human. In certain embodiments, the subject host is a plant.

In some embodiments, the sensor RNA includes the following: (i) a first nucleotide sequence encoding a marker protein, (ii) a second nucleotide sequence encoding a first cleavage domain, (iii) a third nucleotide sequence including a nucleotide sequence that hybridizes to the target RNA, wherein the third nucleotide sequence includes one or more stop codons, (iv) a fourth nucleotide sequence encoding a second cleavage domain, and (v) a fifth nucleotide sequence encoding an output protein.

In some embodiments, the sensor RNA includes the following: (i) a first nucleotide sequence encoding a marker protein, (ii) a second nucleotide sequence encoding a cleavage domain (iii) a third nucleotide sequence including a nucleotide sequence that hybridizes to the 3′ UTR of the target RNA, wherein the third nucleotide sequence includes one or more stop codons, (iv) a fourth nucleotide sequence encoding a second cleavage domain, and (v) a fifth nucleotide sequence encoding an output protein.

In some embodiments, the sensor RNA includes the following: (i) a first nucleotide sequence containing a stem-loop sequence containing one or more stop codons (ii) a second nucleotide sequence containing a nucleotide sequence that hybridizes to the target RNA, (iii) a third nucleotide sequence encoding a cleavage domain, and (iv) a fourth nucleotide sequence encoding an output protein.

In some embodiments, the sensor RNA includes the following: (i) a first nucleotide sequence containing a nucleotide sequence that hybridizes to the target RNA wherein the first nucleotide sequence contains a stem-loop sequence containing one or more stop codons, (ii) a second nucleotide sequence encoding a cleavage domain, and (iii) a third nucleotide sequence encoding an output protein.

In some embodiments, the sensor RNA includes the following: (i) a first nucleotide sequence containing a nucleotide sequence that hybridizes to the target RNA; (ii) a second nucleotide sequence containing a stem-loop sequence containing one or more stop codons; (iii) a third nucleotide sequence encoding a cleavage domain; and (iv) a fourth nucleotide sequence encoding an output protein.

In some embodiments, the sensor RNAs contain a 5′ RNA cap that is 5′ to the first nucleotide sequence. In some embodiments, the sensor RNA contains a sequence encoding a 5′ UTR that is 5′ to the first nucleotide sequence. In some embodiments, the sensor RNA contains a sequence encoding a 5′ UTR that is 5′ to the first nucleotide sequence and a 5′ RNA cap that is 5′ of the sequence encoding the 5′ UTR. In some embodiments, the sensor RNA contains a sequence encoding a 3′ UTR that is 3′ of the sequence encoding the output protein. In some embodiments, the sensor RNA contains a sequence encoding a polyA tail that is 3′ of the sequence encoding the output protein. In some embodiments, the sensor RNA contains a sequencing encoding a 3′ UTR that is 3′ of the sequence encoding the output protein and a sequence encoding a polyA tail that is 3′ of the sequence encoding the polyA tail. In some embodiments, the sensor RNA contains a sequence encoding a 5′ UTR that is 5′ to the first nucleotide sequence and a 5′ RNA cap that is 5′ of the sequence encoding the 5′ UTR, a sequencing encoding a 3′ UTR that is 3′ of the sequence encoding the output protein and a sequence encoding a polyA tail that is 3′ of the sequence encoding the output.

A 5′ cap can be a native 7-methylguanylate cap, or a cap analog, for example anti-reverse cap analog (ARCA), 3′-O-Me-m7G(5′)ppp(5′)G, (m7G(5′)ppp(5′)G), Cap0, Cap1, inosine, N1-methyl-guanosine, 2′ fluoro-guanosine, 7-deaza-guanosine, 8-oxo-guanosine, 2-amino-guanosine, LNA-guanosine, 2-azido-guanosine, etc.

In some embodiments, the sensor RNA has one or more stop codons containing one or more bases that are mismatched with 1) a sequence within the stem-loop opposite the stop codon or 2) a sequence in the target RNA opposite the stop codon. The one or more bases that are mismatched are generally not more than 2 bases that are mismatched. In an embodiment, the sensor RNA has one or more stop codons containing a single base that is mismatched with 1) a sequence within the stem loop opposite the stop codon or 2) a sequence in the target RNA. In some embodiments, the sensor RNA does not have any mismatched bases.

In some instances, the target RNA has one or more base mismatches opposite the stem-loop sequence. There may be a range in the number of bases mismatched opposite the stem-loop sequence including, without limitation, one or more, two or more, three or more, four or more, five or more, six or more, seven or more, eight or more, nine or more, ten or more, or more than ten. In some embodiments, the target RNA has five or more base mismatches opposite the stem-loop sequence, such as ten or more base mismatches opposite the stem-loop sequence. Sensors that base pair in a way that results in the target RNA having mismatches (bulges or loops) opposite the stem-loop sequence of the sensor RNA have advantages over those that do not. The sensor and trigger form a three-way junction, and the presence of an extra bulge or loop at this junction can aid in increased ADAR binding or editing efficiency by providing greater flexibility or optimal positioning.

In some instances, the nucleotide sequence hybridizes to the target or predetermined RNA and the target or predetermined RNA have one or more base mismatches opposite the stem-loop sequence. The one or more base mismatches may be the result of a nucleotide that hybridizes to two discontinuous sequences. For instance, a nucleotide sequence 3′ of the stem-loop sequence hybridizes can hybridize to a 5′ sequence of the target or predetermined RNA and a sensor nucleotide sequence 5′ of a stem-loop sequence can hybridize to a 3′ sequence of the target or predetermined RNA. The 5′ sequence of the target or predetermined RNA and the 3′ sequence of the target or predetermined RNA may be separated by a varying number of nucleotides. For instance, the 5′ sequence of the target r predetermined RNA and the 3′ sequence of the target RNA may be separated by 1 or more, 2 or more, 3 or more, 4 or more, 5 or more, 6 or more, 7 or more, 8 or more, 9 or more, 10 or more, or more than 110 nucleotides.

In some instances, the nucleotide sequence that hybridizes to the target or predetermined RNA has one or more bases mismatched about 25 or more, about 30 or more, about 35 or more, about 40 or more, about 45 or more, about 50 or more, about 55 or more, about 60 or more, about 65 or more, about 70 or more, about 75 or more, about 80 or more, about 85 or more, about 90 or more, about 95 or more, or 100 or more base pairs upstream (e.g. 5′) or downstream (e.g. 3′) of the editable codon. There may be a range in the number of bases mismatched 25 or more base pairs upstream or downstream (e.g. 3′) of the editable codon including, without limitation, one or more, two or more, three or more, four or more, five or more, six or more, seven or more, eight or more, nine or more, ten or more, or more than ten. In some embodiments, the mismatched bases of the nucleotide sequence are 35 or more base pairs upstream or downstream (e.g. 3′) of the editable codon. Sensor sequences that base-pair with a target or predetermined RNA in a way that results in a mismatch 25 or more base pairs upstream or downstream (e.g. 3′) of the editable codon can have advantages over those that do not. Such distant mismatches have, in some instances, been shown to increase ADAR editing efficiency (Uzonyi et al., Molecular Cell 2021; Zambrano-Mila 2023).

In certain embodiments, the sensor RNA contains the first nucleotide sequence to the third, fourth or fifth nucleotide sequences in order (e.g., the fifth nucleotide sequence follows the fourth nucleotide sequence which follows the third nucleotide sequence which follows the second nucleotide sequence which follows the first nucleotide sequence). In some embodiments, the sensor RNA contains the first nucleotide sequence to the third, fourth or fifth nucleotide sequence that are not in order described above.

The sensor RNA of the present disclosure contains a nucleotide sequence that hybridizes to a target RNA or a stem-loop sequence containing one or more stop codons which is followed by a nucleotide sequence encoding an output protein. In some embodiments, the nucleotide sequence that hybridizes to a target RNA contains one or more stop codons that contain at least 1 base that is mismatched with the target or predetermined RNA or the sequence within the stem-loop. In some embodiments, the nucleotide sequence that hybridizes to a target RNA does not contain any mismatches with the target or predetermined RNA. In the presence of the target RNA, the nucleotide sequence that hybridizes to a target RNA can hybridize to the target or predetermined RNA thereby forming a double-stranded RNA molecule that can recruit an ADAR protein. The double-stranded RNA can contain a stop codon with or without mismatches, or a stop codon can be within the stem-loop of the sensor RNA. An ADAR protein can then edit the adenosine base within the stop codon(s) of the sensor RNA to an inosine base. This editing can removes the stop codon(s) which then can allow the output protein to be produced from the sensor RNA within the biological sample.

When the sensor RNA contains a nucleotide sequence containing a stem-loop sequence comprising a stop codon, any stem-loop sequence may be used. In some embodiments, the stem loop contains natural editing sites. Natural editing sites are sites within nucleotide sequences which are edited in nature. Natural editing sites have been described in, for example, Gabay et al. (Nat Commun. 2022 Mar. 4; 13(1):1184) which is specifically incorporated by reference herein. Examples of natural editing sites include, without limitation, editing sites found in GRIA2, GRIA3, IGFBP7, NEIL1, FLNA, GRIK2, CDK13, GABRA3, GLI1, SPEG, HTR2C, GRIA4, CYFIP2, CADPS, CADPS, RICTOR, COG3, GRIK1, COPA, HBE1, SON, FLNB, MAGEL2, NOVA1, PNMT, WASH1, LAT, DACT3, FXYD5, ZNF717, ZNF551 CAPS1, etc. In some embodiments, the stem-loop sequence is a GluR-B stem-loop or a modified variant thereof. In some embodiments, the stem contains a natural editing site while the loop is a synthetic sequence. In some embodiments, the sequence of the stem is altered compared to the natural editing site by the addition or removal of nucleotides in order to add or remove mismatches. In some embodiments, the sequence alteration adds or removes additional stop codons. In some embodiments, the stem-loop sequence contains a CAPS1 derived stem-loop according to a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, nucleotide sequence identity to the following nucleic acid sequence: CAAGGUCAAUGAGGAGAUGUACAUAGAAAUACAAUCCUGUGUACAUCUUCUAGCAU GACCCAC (SEQ ID NO: 1). In some embodiments, the stem-loop sequence contains a CAPS1 derived stem-loop according to a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, nucleotide sequence identity to the following nucleic acid sequence: CAAGGUCAAUGAGGAGAUGUACAUAAUACAAUGUGUACAUCUUCUAGCAUGACCCA C (SEQ ID NO: 2). In some embodiments, the stem-loop sequence contains a GLI1 derived stem-loop according to a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, nucleotide sequence identity to the following nucleic acid sequence: CCCAACCUCUGUCUACUCACCACAGCCCCCCAGCAUCACUGUGAAUGCUGCCAUGGA UGCUAGAGGGCUACAGGAAGAGCCAGAAGUUGG (SEQ ID NO: 3). In some embodiments, the stem-loop sequence contains a GLI1 derived stem-loop according to a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, nucleotide sequence identity to the following nucleic acid sequence: CUCACCACAGCCCCCCAGCAUCACUGUGAAUGCUGCCAUGGAUGCUAGAGGGCUACA GGA (SEQ ID NO: 4). In some embodiments, the stem-loop sequence contains a GABRA3 derived stem-loop according to a nucleic acid having at least 75%, at least 80%, at least 85%, at least 90%, at least 95%, at least 98%, at least 99%, or 100%, nucleotide sequence identity to the following nucleic acid sequence: AAGUGGCAUAUGCGACGGCCAUGGACUGGUUCAUAGCCGUCUGUUAUGCCU (SEQ ID NO: 5). In some embodiments, the stem-loop sequence contains a GABRA3 derived stem-loop according to a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, nucleotide sequence identity to the following nucleic acid sequence: UGGCAUAUGCGACGGCCAUGGACUGGUUCAUAGCCGUCUGUUAUG (SEQ ID NO: 6). In some embodiments, the stem-loop sequence contains a GLURB derived stem-loop according to a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, nucleotide sequence identity to the following nucleic acid sequence: CAUUAAGGUGGGUGGAAUAGUAUACAAAGUAUCCCACCUACCCUGAUG (SEQ ID NO: 7). In some embodiments, the stem-loop sequence contains a GLURB derived stem-loop according to a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, nucleotide sequence identity to the following nucleic acid sequence: CAUUAAGGUGGGUGGAAUAGUAUACAAAGUAUCCCACCUACCCCGAUG (SEQ ID NO: 8). In some embodiments, the stem-loop sequence comprises GLURB derived stem-loop according to a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, nucleotide sequence identity to the following nucleic acid sequence: UCCGUUUAGGUGGGUGGAAUAGUAAUACAAAGUAUCCCACCUACCCAGACG (SEQ ID NO: 9). In some embodiments, the stem-loop sequence is according to a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, nucleotide sequence identity to the following nucleic acid sequence: CAUUUAGGUGGGUGGGCUAACCACCUACCCAGAUG (SEQ ID NO: 10). In some embodiments, the stem-loop sequence is according to a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, nucleotide sequence identity to the following nucleic acid sequence: CAUUUAGGUGGGUGGAAUGCUAAAUCCCACCUACCCAGAUG (SEQ ID NO: 11). In some embodiments, the stem-loop sequence is according to a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, nucleotide sequence identity to the following nucleic acid sequence:

(SEQ ID NO: 12)
AUGCGACGGCCAUGGACUGGUUCAUAGCCGUCUGUU.

When the sensor RNA contains a nucleotide sequence containing a stem-loop sequence containing a stop codon, the length of the stem-loop may have a limit. For example, the stem-loop may be 50 bp or less, 45 bp or less, 40 bp or less, 35 bp or less, 30 bp or less, 25 bp or less, or 20 bp or less. In an embodiment, the length of the stem-loop is 18-50 bps.

In some embodiments, the stem-loop sequence, the sequence that hybridizes to the target or predetermined RNA, the sensor RNA (e.g. between any of the two aforementioned elements)comprises one or more stop codons that are out of frame of the editable codon. In some embodiments, the stem-loop sequence, the sensor RNA, or the sequence that hybridizes to the target or predetermined RNA comprises two or more stop codons that are out of frame of the editable codon. In some embodiments, the two or more stop codons that are out of frame are defined by CUAAAUAAA (SEQ ID NO: 13). Other sequences may be employed for the two or more stop codons out of frame with the editable codon. The other sequences abide by the following: 1) any base, 2) a stop codon (UAG, UGA, UAA), 3) any base, 4) a stop codon (UAG, UGA, UAA), and 5) any base. For instance, the sequence may be NUAGNUAG, NUAGNUGA, NUAGNUAA, NUGANUAG, NUGANUGA, NUGANUAA, NUAANUAG, NUAANUGA, or NUAANUAA where N is equivalent to any base. Another sequence may be chosen if the three amino acid peptide encoded by the sequence is better suitable for expression in the reading frame of the editable codon. Sensors containing out of frame stop codons have certain advantages relative to sensor RNAs that do not contain such codons, as these stop codons can halt translation when the ribosome has shifted frames which can lead to skipping of the editable codon in its correct frame and thus loss of translational control. Continuing translation in the wrong frame can also cause unwanted protein products that can have detrimental effects. Sensors containing out of frame stop codons within the stem-loop, particularly in the loop portion, have advantages relative to sensor RNAs containing out of frame stop codons elsewhere. RNA structures interact with ribosomes, and having a strong secondary structure in the form of a stem-loop can make the RNA structures more predictable. Placing the stop codons in the loop may result in more efficient reading of those stops, rather than readthrough. In some embodiments, the first cleavage domain contains out-of-frame stop codons. In some embodiments, the second cleavage domain contains out-of-frame stop codons. In some embodiments, the out-of-frame stop codons are in the +1 or +2 frame. In some embodiments, the out-of-frame stop codons are in the +1 frame. In some embodiments, the out-of-frame stop codons are in the +2 frame. In some embodiments, the first cleavage domain is a 2A cleavage sequence that is re-coded to contain one or more out-of-frame stop codons. By “re-coded” it is meant that the sequence encoding the 2A cleavage site is altered such that it contains an out-of-frame stop codon but still encodes a functional 2 cleavage sequence. In some embodiments, the second cleavage domain is a 2A cleavage sequence that is re-coded to contain one or more out-of-frame stop codons. In some embodiments, the 2A cleavage sequence is a T2A cleavage sequence. In some embodiments, the 2A cleavage sequence is a P2A cleavage sequence. In some embodiments, the 2A cleavage sequence is an E2A cleavage sequence. In some embodiments, the 2A cleavage sequence is a F2A cleavage sequence.

Sensor RNAs containing a nucleotide sequence containing a stem-loop sequence containing an editable codon can have certain advantages relative to sensor RNAs that do not contain such a stem-loop sequence. This is due to ADAR having separate domains for RNA editing (catalytic domain) and dsRNA binding. First, sensor RNAs containing a nucleotide sequence containing a stem-loop sequence containing an editable codon decouples the sequence that is being edited (e.g., a stop codon) from the sequence that recruits the ADAR protein (e.g., the dsRNA segment that is formed when the nucleotide sequence that hybridizes to the target RNA hybridizes to the target RNA). Generally, if the editable codon in the sensor RNA is a UAG (stop codon) and there is a single mismatch in the stop codon relative to the target RNA then the corresponding target RNA has a CCA sequence (or a sequence that hybridizes to a different stop codon having one mismatch with the stop codon). The presence of the CCA sequence (or an equivalent sequence for a different editable codon) potentially limits the number of possible target RNAs. Requiring a specific sequence (such as CCA or an equivalent sequence) to be present in the target RNA can may limit the subsequences a sensor can be utilized to detect; for example, a CCA or equivalent sequence may be present in highly structured parts of the target RNA, may be present in the coding sequence, or may be present in protein-bound sections of a target RNA, all of which may contribute to lower availability for sensor-target hybridization, reducing efficiency. With a sensor containing a stem-loop, the range of suitable subsequences is greatly increased, so problematic target RNA subsequences can be avoided and efficient ones utilized instead. In some embodiments where gene fusions or splice variants are to be distinguished, flexibility in target RNA subsequence can be employed, and is provided by the stem-loop design. Second, while ADAR editing is largely sequence-agnostic, there are some minor biases primarily driven by the catalytic domain which extend beyond the editable codon. Biases driven by the catalytic domain have been described by, for example, Kuttan et al. (Proc Natl Acad Sci USA. 2012 Nov. 27; 109(48):E3295-304) which is specifically incorporated by reference herein. Editing sites in the sensor RNA may be dictated by the target RNA which precludes optimization of the editing site (e.g., the stop or non-stop codons of the present disclosure). By separating out the editing site from the nucleotide sequence that hybridizes to the target RNA, to the editing site and the nucleotide sequence can be optimized separately.

In some embodiments, the sensor RNA contains a non-start codon in place of a stop codon. In these embodiments, the sensor RNA contains the following: (i) a first nucleotide sequence containing a nucleotide sequence that hybridizes to the target RNA, wherein the first nucleotide sequence contains a non-start codon (e.g. AUA) that contains at least 1 base that is mismatched with the target RNA sequence, (ii) a second nucleotide sequence encoding a second cleavage domain, and (iii) a third nucleotide sequence encoding an output protein. In the presence of the target RNA, the sensor RNA hybridizes to the target RNA thereby forming a double stranded RNA molecule containing one or more base mismatches within the non-start codon or elsewhere. An ADAR protein then edits the adenosine base within the non-start codon (e.g., AUA to AUI) of the sensor RNA to an inosine base. This editing converts the non-start codon to a start codon which then allows the output protein to be produced from the sensor RNA within the biological sample.

In some embodiments, the sensor RNA has a start codon in place of a stop codon. In these embodiments, the sensor RNA has the following: (i) a first nucleotide sequence having a nucleotide sequence that hybridizes to the target RNA, wherein the first nucleotide sequence has a start codon (e.g. AUG) that has at least 1 base that is mismatched with the target RNA sequence and (ii) a second nucleotide sequence encoding an output protein wherein the sequence encoding the output protein has a start codon. In the presence of the target RNA, the sensor RNA hybridizes to the target RNA thereby forming a double stranded RNA molecule having one or more base mismatches within the start codon or elsewhere. An ADAR protein then edits the adenosine base within the start codon (e.g., AUG to IUG) of the sensor RNA to an inosine base. This editing converts the start codon to a non-start codon which then allows the output protein to be produced from the sensor RNA within the biological sample. Prior to editing, the presence of the first start codon within the first nucleotide sequence can represent an upstream (e.g. 5′) reading frame which suppresses the expression of the downstream (e.g. 3′) reading frame. After editing, the upstream (e.g. 5′) reading frame can be removed allowing the downstream (e.g. 3′) reading frame to be expressed which produces the output protein.

When the sensor RNA contains a start codon in place of a stop codon, the upstream (e.g. 5′) reading frame, e.g., as described above, may have particular features. In some embodiments, the length of the upstream (e.g. 5′) reading frame is shorter than the downstream (e.g. 3′) reading frame. In some embodiments, the length of the upstream (e.g. 5′) reading frame is longer than the downstream (e.g. 3′) reading frame. In an embodiment, the length of the upstream (e.g. 5′) reading frame is about the same length as the length of the downstream (e.g. 3′) reading frame.

In some embodiments, the sensor RNA contains a start codon in place of a stop codon. In these embodiments, the sensor RNA contains the following: (i) a first nucleotide sequence containing a nucleotide sequence that hybridizes to the target RNA, wherein the first nucleotide sequence contains a start codon (e.g., AUG) that contains at least 1 base that is mismatched with the target RNA sequence and (ii) a second nucleotide sequence encoding an output protein. In the presence of the target RNA, the sensor RNA hybridizes to the target RNA thereby forming a double-stranded RNA molecule containing one or more base mismatches within the start codon or elsewhere. An ADAR protein then edits the adenosine base within the start codon (e.g., AUG to IUG) of the sensor RNA to an inosine base. This editing converts the start codon to a non-start codon which then prevents the production of the output protein. In this embodiment, the output protein is produced in the absence of the target RNA (e.g. is selectively produced in the absence of the target RNA).

In some embodiments, the sensor RNA includes splice sites upstream of (e.g. 5′ to) the output protein. In these embodiments, the ADAR protein edits a codon at the splice thereby removing the splice site leading to the production of the output protein. In some embodiments, the ADAR protein edits a non-splice site converting it into a splice site thereby inactivating the production of the output protein.

In some cases, it is advantageous to reduce the immunogenicity of the sensor RNA. Methods of reducing the immunogenicity of RNAs have been described by, for example, Starostina et al. (Vaccines (Basel). 2021 May 3; 9(5):452) which is specifically incorporated by reference herein. In general, methods of reducing the immunogenicity of a sensor RNA involve the incorporation of modified ribonucleic acids into the sensor RNA. Modified ribonucleic acids that find use in the present disclosure includes, without limitation, pyridin-4-one ribonucleoside, 5-aza-uridine, 2-thio-5-aza-uridine, 2-thiouridine, 4-thio-pseudouridine, 2-thio-pseudouridine, 5-hy-droxyuridine, 3-methyluridine, 5-carboxymethyluridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thiouridine, 1-tau-rinomethy1-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine. 2-thio-dihydrouridine, 2-thiodihydropseudouridine, 2-methoxyuridine, 2-methoxy-4-thiouridine, 4-methoxy-pseudouridine, 4-methoxy-2-thio-pseudouridine, 5-azacytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2, 6-diaminopurine, 1-methyladenosine, N6-methyladenosine, N6-isopentenyladenosine, N6-(cis-hydroxyisopentenyadenosine, 2-methylthio-N-6-(cis-hydroxyisopentenyl) adenosine, N6-glycinylcarbamoyladenosine, N6-threonyl-carbamoyladenosine, 2-methylthio-N6-threonyl carbamoyladenosine, N6.N6-dimethyladenosine, 7-methyladenine, 2-methylthio-adenine, 2-methoxy-adenine, inosine, 1-me-thyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylgua-nosine, N2,N2-dimethylguanosine, 8-oxo-guanosine, 7-me-thy1-8-oxo-guanosine, 1-methyl-6-thio-guanosine, N2-methyl-6-thio-guano sine, N2,N2-dimethyl-6-thio-guanosine, methylcytosine, pseudouridine, methyladenosine, etc. In some embodiments, the methylcytosine is 5-methylcytosine. In some embodiments, the pseudouridine is N1-methyl-pseudouridine. In some embodiments, the methyladenosine is a N6-methyladenosine. In some embodiments, the methyladenosine is a N1-methyladenosine. In some embodiments, a portion of the nucleotides present in the sensor RNA are composed of modified ribonucleic acids. For instance, a portion of the uridines in the sensor RNA are replaced with pseudouridines. When the uridines of the sensor RNA are replaced with pseudouridines, a certain percentage of the uridines are replaced with pseudouridines. For instance, about 1-10%, about 10-20%, about 20-30%, about 30-40%, about 40-50%, about 50-60%, about 60-70%, about 70-80%, 80-90% or greater than 90% of the uridines are replaced with pseudouridines. In an embodiment, 75% or less of the uridines in the sensor RNAs are replaced with pseudouridines. In an embodiment, the sensor sequence or parts of it do not have pseudouridines.

When a sensor RNA contains pseudouridines, the pseudouridine(s) may be in specific locations. In some embodiments, the pseudouridine(s) are not adjacent to adenosines that are the targets of ADAR editing. In some embodiments, the pseudouridine(s) are not contained in the sensor sequence that hybridizes with a target RNA. When a sensor RNA contains pseudouridines, the sensor may contain a particular stop codon. In some embodiments, the stop codon used is UGA. When the UGA stop codon is used, the adenosine in the UGA may be followed by a specific nucleotide. In some embodiments, the adenosine in the UGA is followed by guanosine such the nucleotide sequence is UGAG.

In some embodiments, the nucleotide sequence that hybridizes to the predetermined or target RNA includes bases that are mismatched with adenosine bases within the target or predetermined RNA that are not within a start or stop codon. In some embodiments, the mismatched bases prevent the editing of adenosines that are not within the stop or start codons.

In some embodiments, the nucleotide sequence that hybridizes to the predetermined or target RNA includes one or more editing inducing elements (EIEs). Suitable EIEs that find use in the present disclosure are disclosed within Uzonyi et al. (Mol Cell. 2021 Jun. 3; 81(11):2374-2387) and Danan-Gotthold et al. (Genome Biol. 2017 Oct. 23; 18(1):196).

A marker protein of the present disclosure may be any marker protein that is useful for the detection of the presence of a sensor mRNA within a biological sample. For instance, the marker protein may be a fluorescent protein or a luminescent protein. Non-limiting examples of useful fluorescent proteins include but are not limited to GFP, EBFP, Azurite, Cerulean, mCFP, Turquoise, ECFP, mKeima-Red, TagCFP, AmCyan, mTFP, TurboGFP, TagGFP, EGFP, TagYFP, EYFP, Topaz, Venus, mCitrine, TurboYFP, mOrange, TurboRFP, tdTomato, TagRFP, dsRed2, mRFP, mCherry, mPlum mRaspberry, mScarlet, etc. Examples of luminescent proteins include without limitation, Cypridinia luciferase, Gaussia luciferase, Renilla luciferase, Phontinus luciferase, Luciola luciferase, Pyrophorus luciferase, Phrixothrix luciferase, etc. In some embodiments, the marker protein may be the first half of the output protein. In these embodiments, the sequence encoding the marker protein produces the first half of the output which may be non-functional without the second half of the output protein in the absence of the target RNA. In the presence of the target RNA, the second half of the output protein is produced. When the second half of the output protein is produced in the presence of the first half of the output protein, the two halves are then able to form a functional output protein. In these embodiments, the first half of the output protein is the N-terminus of the output protein and the second half of the output protein is the C-terminus of the output protein.

In certain embodiments, the sensor RNA includes a nucleotide sequence that encodes a cleavage domain. Cleavage domains that find use in the present disclosure include without limitation, HIV-1 protease cleavage domain, TEV cleavage domain, preScission protease cleavage domain, HCV protease cleavage domain, RecA cleavage domain, self-cleaving domain, etc. When a self-cleaving domain is used then the self-cleaving domain may be a 2A self-cleaving domain. 2A self-cleaving domains that find use in the present disclosure include T2A, P2A, E2A and F2A which are described in Szymczak-Workman et al. (Cold Spring Harb Protoc. 2012 Feb. 1; 2012(2):199-204). In some embodiments, the sensor RNA includes a first and a second cleavage domain. When the sensor RNA includes a first and a second cleavage domain, the cleavage domains may be of the same type or they may be of a different type. For instance, the first cleavage domain may be a P2A self-cleaving domain and the second cleavage domain may also be a P2A self-cleaving domain or the first cleavage domain may be a P2A self-cleaving domain and the second cleavage domain may be a T2A self-cleaving domain or any combination thereof.

The nucleotide sequence of the present disclosure that hybridizes to the predetermined or target RNA may be hybridized to any region of the target or predetermined RNA. In certain embodiments, the nucleotide sequence hybridizes to the 3′ UTR of the target or predetermined RNA. In some embodiments, the nucleotide sequence hybridizes to the 5′ UTR of the target or predetermined RNA. In certain embodiments, the nucleotide sequence hybridizes to the coding sequence of the target or predetermined RNA. In some embodiments, the nucleotide sequence hybridizes to an exon of the target or predetermined RNA. In some embodiments, the nucleotide sequence hybridizes to an intron of the target or predetermined RNA. In some embodiments, the nucleotide sequence that hybridizes to the predetermined or target RNA hybridizes to two separate non-contiguous regions of the same target or predetermined RNA. For instance, the nucleotide sequence may hybridize to two separate regions of the 5′ UTR of the target RNA, to two separate regions of the coding sequence of the target RNA, to two separate regions of the 5′ UTR of the target RNA, to a region in the 5′ UTR and a region in the coding sequence of the target RNA, to a region in the coding sequence and a region in the 3′ UTR of the target RNA, or to a region in the 5′ UTR and a region in the 3′ UTR of the target RNA. In some embodiments, the nucleotide sequence that hybridizes to the predetermined or target RNA hybridizes to two or more distinct target or predetermined RNAs.

Sensor RNAs that have nucleotide sequences that hybridize to the 3′ or 5′ UTR can have certain advantages relative to sensor RNAs that hybridize to coding sequences (CDS). First, ADAR editing is more efficient in the UTR when compared CDS because translating ribosomes may destabilize dsRNA. Second, RADAR is less likely to interfere with the production of the protein encoded by the target RNA because 1) dsRNA formation in the UTR rather than the CDS will not affect the translation ribosome, and 2) any bystander editing that occur in the UTR of the target RNA is less likely to cause detrimental outcomes because this region is outside the coding sequence.

The region that hybridizes to the target or predetermined RNA within the sensor RNA of the present disclosure may be any length that provides specificity (e.g. of hybridization) to the target or predetermined RNA. For instance region that hybridizes to the target RNA can be less than about 50 nucleotides, from about 50 to 60, about 60 to 70, about 70 to 80, about 80 to 90, about 90 to 100, about 100 to 110, about 110 to 120, about 120 to 130, about 130 to 140, about 140 to 150, about 150 to 160, about 160 to 170, about 170 to 180, about 180 to 190, about 190 to 200, about 200 to 210, about 210 to 220, about 220 to 230, about 230 to 240, about 240 to 250, about 250 to 260, about 260 to 270, about 270 to 280, about 280 to 290, about 290 to 300, about 300 to 310, about 310 to 320, about 320 to 330, about 330 to 340, about 340 to 350, about 350 to 360, about 360 to 370, about 370 to 380, about 380 to 390, about 390 to 400, about 400 to 410, about 410 to 420, about 420 to 430, about 430 to 440, about 440 to 450, about 450 to 460, about 460 to 470, about 470 to 480, about 480 to 490, about 490 to 500 or greater than 500 nucleotides in length. In some cases, the region that hybridizes to the target RNA can be greater than or equal to about 30 nucleotides, about 35 nucleotides, about 40 nucleotides, about 50 nucleotides, about 55 nucleotides, about 60 nucleotides, about 65 nucleotides, about 70 nucleotides, about 75 nucleotides, about 80 nucleotides, about 85 nucleotides, about 90 nucleotides, about 95 nucleotides, about 100 nucleotides, about 102 nucleotides, about 105 nucleotides, about 110 nucleotides, about 115 nucleotides, about 120 nucleotides, about 125 nucleotides, about 130 nucleotides, about 135 nucleotides, about 140 nucleotides, about 145 nucleotides, about 150 nucleotides, about 155 nucleotides, about 160 nucleotides, about 165 nucleotides, about 170 nucleotides, about 180 nucleotides, about 185 nucleotides about 190 nucleotides, about 195 nucleotides, about 200 nucleotides, about 210 nucleotides, about 220 nucleotides, about 230 nucleotides, about 240 nucleotides, about 250 nucleotides, about 260 nucleotides, about 270 nucleotides, about 280 nucleotides, about 290 nucleotides, about 300 nucleotides, about 310 nucleotides, about 320 nucleotides, about 330 nucleotides, about 340 nucleotides, about 350 nucleotides, about 360 nucleotides, about 370 nucleotides, about 380 nucleotides, about 390 nucleotides, about 400 nucleotides, or any range between these values. In some cases, the region that hybridizes to the target RNA can be less than or than equal to about 35 nucleotides, about 40 nucleotides, about 50 nucleotides, about 55 nucleotides, about 60 nucleotides, about 65 nucleotides, about 70 nucleotides, about 75 nucleotides, about 80 nucleotides, about 85 nucleotides, about 90 nucleotides, about 95 nucleotides, about 100 nucleotides, about 102 nucleotides, about 105 nucleotides, about 110 nucleotides, about 115 nucleotides, about 120 nucleotides, about 125 nucleotides, about 130 nucleotides, about 135 nucleotides, about 140 nucleotides, about 145 nucleotides, about 150 nucleotides, about 155 nucleotides, about 160 nucleotides, about 165 nucleotides, about 170 nucleotides, about 180 nucleotides, about 185 nucleotides about 190 nucleotides, about 195 nucleotides, about 200 nucleotides, about 210 nucleotides, about 220 nucleotides, about 230 nucleotides, about 240 nucleotides, about 250 nucleotides, about 260 nucleotides, about 270 nucleotides, about 280 nucleotides, about 290 nucleotides, about 300 nucleotides, about 310 nucleotides, about 320 nucleotides, about 330 nucleotides, about 340 nucleotides, about 350 nucleotides, about 360 nucleotides, about 370 nucleotides, about 380 nucleotides, about 390 nucleotides, about 400 nucleotides, or any range between these values.

When the region that hybridizes to the target or predetermined RNA comprises two regions that hybridize to two non-contiguous regions within a target RNA, the distance between the two non-contiguous regions of the target may be any length. For instance, the distance between the two non-contiguous regions of the target may be less than about 50 nucleotides, from about 50 to 60, about 60 to 70, about 70 to 80, about 80 to 90, about 90 to 100, about 100 to 150, about 150 to 200, about 200 to 250, about 250 to 300, about 300 to 350, about 350 to 400, about 400 to 450, about 450 to 500 or greater than 500 nucleotides.

The region that hybridizes to the target or predetermined RNA within the sensor RNA of the present disclosure may be any percentage identity to the target or predetermined RNA that provides specificity (e.g. of hybridization) to the target or predetermined RNA. For instance, the region that hybridizes to the target or predetermined RNA can comprise a sequence having at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% nucleotide sequence identity

When the region that hybridizes to the target RNA comprises two regions that hybridize to two non-contiguous regions within the target RNA, the nucleotide sequence of the that hybridizes to the first region of the two non-contiguous regions with the target RNA may be any length. For instance, the region that hybridizes to the first region of the two non-contiguous regions may be less than about 20 nucleotides, from about 20 to 30, about 30 to 40, about 40 to 50, about 50 to 60, about 60 to 70, about 70 to 80, about 80 to 90, about 90 to 100, about 100 to 110, about 110 to 120, about 120 to 130, about 130 to 140, about 140 to 150, about 150 to 160, about 160 to 170, about 170 to 180, about 180 to 190, about 190 to 200, about 200 to 210, about 210 to 220, about 220 to 230, about 230 to 240, about 240 to 250, about 250 to 260, about 260 to 270, about 270 to 280, about 280 to 290, about 290 to 300, about 300 to 310, about 310 to 320, about 320 to 330, about 330 to 340, about 340 to 350, about 350 to 360, about 360 to 370, about 370 to 380, about 380 to 390, about 390 to 400, about 400 to 410, about 410 to 420, about 420 to 430, about 430 to 440, about 440 to 450, about 450 to 460, about 460 to 470, about 470 to 480, about 480 to 490, about 490 to 500 or greater than 500 nucleotides in length.

When the region that hybridizes to the target RNA comprises two regions that hybridize to two non-contiguous regions with the target RNA, the nucleotide sequence of the region that hybridizes that hybridizes to the second region of the two non-contiguous regions with the target RNA may be any length. For instance, the nucleotide sequence of the sensor nucleotide that hybridizes to the second region of the two non-contiguous regions may be less than about 20 nucleotides, from about 20 to 30, about 30 to 40, about 40 to 50, about 50 to 60, about 60 to 70, about 70 to 80, about 80 to 90, about 90 to 100, about 100 to 110, about 110 to 120, about 120 to 130, about 130 to 140, about 140 to 150, about 150 to 160, about 160 to 170, about 170 to 180, about 180 to 190, about 190 to 200, about 200 to 210, about 210 to 220, about 220 to 230, about 230 to 240, about 240 to 250, about 250 to 260, about 260 to 270, about 270 to 280, about 280 to 290, about 290 to 300, about 300 to 310, about 310 to 320, about 320 to 330, about 330 to 340, about 340 to 350, about 350 to 360, about 360 to 370, about 370 to 380, about 380 to 390, about 390 to 400, about 400 to 410, about 410 to 420, about 420 to 430, about 430 to 440, about 440 to 450, about 450 to 460, about 460 to 470, about 470 to 480, about 480 to 490, about 490 to 500 or greater than 500 nucleotides in length.

The sensor nucleotide sequence or the stem-loops of the present disclosure may include any stop or start codon including an adenosine residue. For example, the stop codon of the sensor RNA or the stem-loops may be UAG, UAA, or UGA. In general, the stop codons of the present disclosure can be in-frame with the coding sequence of the output protein such that the output protein is produced when the stop codon is edited.

The output protein of the present disclosure may be any predetermined output protein. Examples of the output protein of the present disclosure include, without limitation, a fluorescent protein, a genomic modification protein, a transcription factor, a killing factor, a toxin, an antigen, a T cell receptor, an enzyme, a therapeutic protein, a cytokine, a chemokine, a growth factor, a signaling peptide, a chimeric antigen receptor (CAR), etc. The output proteins may be secreted, transmembrane or membrane-tethered. When output proteins are to be trafficked to specific locations within the biological sample then the coding sequence of the output protein is preceded by a nucleotide sequence encoding the appropriate signal peptide such as those described in Owji et al. (Eur J Cell Biol. 2018 Aug; 97(6):422-441).

When the output protein is a genomic modification protein, the genomic modification proteins may include, without limitation, CRE recombinase or variants thereof, meganucleases or variants thereof, Zinc-finger nucleases or variants thereof, CRISPR/Cas-9 nuclease or variants thereof, a modified Cas9 nickase fused to a reverse-transcriptase (e.g., genomic modification protein used in prime editing), TAL effector nucleases or variants thereof, etc. Methods of prime editing have been described in, for example, Scholefield et al (Gene Ther. 2021 August; 28(7-8):396-401) which is specifically incorporated by reference herein.

When the output protein is a transcription factor, the transcription factor may include, without limitation, jun, fos, max, mad, serum response factor (SRF), AP-1, AP2, myb, MyoD, myogenin, ETS-box containing proteins, TFE3, E2F, ATF1, ATF2, ATF3, ATF4, ZF5, NFAT, CREB, 5 HNF4, C/EBP, SP1, CCAAT-box binding proteins, interferon regulation factor (IRF-1), Wilms tumor protein, ETS-binding protein, STAT, GATA-box binding proteins, e.g., GAT A-3, and the forkhead family of winged helix proteins.

When the output protein is a killing factor, the killing factor may include, without limitation, tumor necrosis factor alpha (TNFa), Fas ligand (FasL), a caspase such as caspase 1, caspase 2, caspase 3, caspase 4, caspase 5, caspase 6, caspase 7, caspase 8, caspase 9, caspase 10, caspase 11, caspase 12, caspase 13 or a variant thereof, etc.

When the output protein is a therapeutic protein, the therapeutic protein of may include, without limitation, hormones and growth and differentiation factors including, without limitation, insulin, glucagon, growth hormone (GH), parathyroid hormone (PTH), growth hormone releasing factor (GHRF), follicle stimulating hormone (FSH), luteinizing hormone (LH), human chorionic gonadotropin (hCG), vascular endothelial growth factor (VEGF), angioproteinetins, angiostatin, granulocyte colony stimulating factor (GCSF), erythroproteinetin (EPO), connective tissue growth factor (CTGF), basic fibroblast growth factor (bFGF), acidic fibroblast growth factor (aFGF), epidermal growth factor (EGF), transforming growth factor alpha. (TGFa), platelet-derived growth factor (PDGF), insulin growth factors I and II (IGF-1 and IGF-11), any one of the transforming growth factor 13-superfamily, including TGFI3, activins, inhibins, or any of the bone morphogenic proteins (BMP) including BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

When the output protein is a cytokine, the cytokine may include, without limitation IL-1-like, IL-1α, IL-1β, IL-1RA, IL-18, CD132, IL-2, IL-4, IL-7, IL-9, IL-13, CD1243, 132, IL-15, CD131, IL-3, IL-5, GM-CSF, IL-6-like, IL-6, IL-11, G-CSF, IL-12, LIF, OSM, IL-10-like, IL-10, IL-20, IL-14, IL-16, IL-17, IFN-α, IFN-β, IFN-γ, CD154, LT-β, TNF-α, TNF-β, 4-1BBL, APRIL, CD70, CD153, CD178, GITRL, LIGHT, OX40L, TALL-1, TRAIL, TWEAK, TRANCE, TGF-β1, TGF-β2, TGF-β3, Epo, Tpo, Flt-3L, SCF, M-CSF, MSP, etc.

When the output protein is a chemokine, the chemokine may include, without limitation XCL1, XCL2, CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CXCL1, CXCL2, CXCL3, CXCL4, CXCL5, CXCL6, CXCL7, CXCL8, CXCL9, CXCL10, CXCL11, CXCL12, CXCL13, CXCL14, CX3CL1, etc.

In certain embodiments, when the output polypeptide is a CAR, the extracellular binding domain of the CAR has a single chain antibody. The single-chain antibody may be a monoclonal single-chain antibody, a chimeric single-chain antibody, a humanized single-chain antibody, or a fully human single-chain antibody. In one non-limiting example, the single chain antibody is a single chain variable fragment (scFv). Suitable CAR extracellular binding domains include those described in Labanieh et al. (2018 Nature Biomedical Engineering 2:377-391) which is specifically incorporated by reference herein. In some embodiments, the extracellular binding domain of the CAR is a single-chain version (e.g., an scFv version) of an antibody approved by the United States Food and Drug Administration or the European Medicines Agency (EMA) for use as a therapeutic antibody, e.g., for inducing antibody-dependent cellular cytotoxicity (ADCC) of certain disease-associated cells in a patient, etc. Non-limiting examples of single-chain antibodies which may be employed when the protein of interest is a CAR include single-chain versions (e.g., scFv versions) of Adecatumumab, Ascrinvacumab, Cixutumumab, Conatumumab, Daratumumab, Drozitumab, Duligotumab, Durvalumab, Dusigitumab, Enfortumab, Enoticumab, Figitumumab, Ganitumab, Glembatumumab, Intetumumab, Ipilimumab, Iratumumab, Icrucumab, Lexatumumab, Lucatumumab, Mapatumumab, Narnatumab, Necitumumab, Nesvacumab, Ofatumumab, Olaratumab, Panitumumab, Patritumab, Pritumumab, Radretumab, Ramucirumab, Rilotumumab, Robatumumab, Seribantumab, Tarextumab, Teprotumumab, Tovetumab, Vantictumab, Vesencumab, Votumumab, Zalutumumab, Flanvotumab, Altumomab, Anatumomab, Arcitumomab, Bectumomab, Blinatumomab, Detumomab, Ibritumomab, Minretumomab, Mitumomab, Moxetumomab, Naptumomab, Nofetumomab, Pemtumomab, Pintumomab, Racotumomab, Satumomab, Solitomab, Taplitumomab, Tenatumomab, Tositumomab, Tremelimumab, Abagovomab, Igovomab, Oregovomab, Capromab, Edrecolomab, Nacolomab, Amatuximab, Bavituximab, Brentuximab, Cetuximab, Derlotuximab, Dinutuximab, Ensituximab, Futuximab, Girentuximab, Indatuximab, Isatuximab, Margetuximab, Rituximab, Siltuximab, Ublituximab, Ecromeximab, Abituzumab, Alemtuzumab, Bevacizumab, Bivatuzumab, Brontictuzumab, Cantuzumab, Cantuzumab, Citatuzumab, Clivatuzumab, Dacetuzumab, Demcizumab, Dalotuzumab, Denintuzumab, Elotuzumab, Emactuzumab, Emibetuzumab, Enoblituzumab, Etaracizumab, Farletuzumab, Ficlatuzumab, Gemtuzumab, Imgatuzumab, Inotuzumab, Labetuzumab, Lifastuzumab, Lintuzumab, Lorvotuzumab, Lumretuzumab, Matuzumab, Milatuzumab, Nimotuzumab, Obinutuzumab, Ocaratuzumab, Otlertuzumab, Onartuzumab, Oportuzumab, Parsatuzumab, Pertuzumab, Pinatuzumab, Polatuzumab, Sibrotuzumab, Simtuzumab, Tacatuzumab, Tigatuzumab, Trastuzumab, Tucotuzumab, Vandortuzumab, Vanucizumab, Veltuzumab, Vorsetuzumab, Sofituzumab, Catumaxomab, Ertumaxomab, Depatuxizumab, Ontuxizumab, Blontuvetmab, Tamtuvetmab, or an antigen-binding variant thereof.

The output protein may further include a tag to be used to detect the protein following its production. For instance, the tag may include, without limitation, a fluorescent protein, e.g., green fluorescent protein (GFP), YFP, RFP, CFP, mCherry, tdTomato, and the like; a histidine tag, e.g., a 6×His tag; a hemagglutinin (HA) tag; a FLAG tag; a Myc tag; and the like.

Aspects of this disclosure include assaying for the presence of the output protein in a biological sample. In some embodiments, the assaying for the output protein may contain using immunoblotting. In some embodiments, the assaying contains using microscopy. When the assaying contains microscopy the output protein may be conjugated to a fluorescent or luminescent protein or the output protein may be a fluorescent or luminescent protein. In some embodiments, the assaying for the presence of the output protein contains using flow cytometry. When the assaying includes flow cytometry, fluorescence-activating cell sorting may be used. In some embodiments, the assaying for the presence of the output protein comprises using a plate reader.

The methods of the present disclosure also contain combining the biological sample with the sensor RNA. The combining can be done using any convenient method. In some embodiments, the combining includes transfecting the biological sample with a recombinant vector containing the sensor RNA. When the biological sample is transfected with the recombinant vector, the recombinant vector includes, without limitation, a plasmid, a viral vector, a cosmid an artificial chromosome, etc. In some embodiments, the combining contains contacting the biological sample with a lipid nanoparticle containing the sensor RNA. Lipid nanoparticles have been described in the art such as Hou et al. (Nat Rev Mater. 2021; 6(12):1078-1094).

When transfection of a biological sample such as a cell is advantageous, vectors, such as plasmids viral vectors, cosmids or artificial chromosomes, may be employed to engineer the cell to express the sensor RNA. Protocols of interest include those described in published PCT application WO1999/041258, the disclosure of which protocols are herein incorporated by reference.

Depending on the nature of the cell or expression construct, protocols of interest may include electroporation, particle gun technology, calcium phosphate precipitation, direct microinjection, viral infection and the like. The choice of method is generally dependent on the type of cell being transformed and the circumstances under which the transformation is taking place (e.g., in vitro, ex vivo, or in vivo). A general discussion of these methods can be found in Ausubel, et al, Short Protocols in Molecular Biology, 3rd ed., Wiley & Sons, 1995. In some embodiments, lipofectamine and calcium mediated gene transfer technologies are used. After the subject nucleic acids have been introduced into a cell, the cell may be incubated, normally at 37° C., sometimes under selection, for a period of about 1-24 hours in order to allow for the expression of the sensor RNA. In mammalian target cells, a number of viral-based expression systems may be utilized to express the sensor RNA(s). In cases where an adenovirus is used as an expression vector, the sensor RNA sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the chimeric protein in infected hosts. (e.g., see Logan & Shenk, Proc. Natl. Acad. Sci. USA 81:355-359 (1984)). The efficiency of expression may be enhanced by the inclusion of appropriate transcription enhancer elements, transcription terminators, etc. (see Bittner et al., Methods in Enzymol. 153:51-544 (1987)).

In some embodiments, the viral vector is a recombinant adeno-associated virus (AAV) vector. AAV vectors are DNA viruses of relatively small size that can integrate, in a stable and site-specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

The application of AAV as a vector for gene therapy has been rapidly developed in recent years. Wild-type AAV can infect, with a comparatively high titer, dividing or non-dividing cells, or tissues of mammal, including human, and also can integrate into in human cells at specific site (on the long arm of chromosome 19) (Kotin et al, Proc. Natl. Acad. Sci. U.S.A., 1990. 87: 2211-2215; Samulski et al, EMBO J., 1991. 10: 3941-3950 the disclosures of which are hereby incorporated by reference herein in their entireties). AAV vector without the rep and cap genes loses specificity of site-specific integration, but may still mediate long-term stable expression of exogenous genes. AAV vector exists in cells in two forms, wherein one is episomic outside of the chromosome; another is integrated into the chromosome, with the former as the major form. Moreover, AAV has not been found to be associated with any human disease, nor any change of biological characteristics arising from the integration has been observed. There are sixteen serotypes of AAV reported in literature, respectively named AAV1, AAV2, AAV3, AAV4, AAV5, AAV6, AAV7, AAV8, AAV9, AAV10, AAV11, AAV12, AAV13, AAV14, AAV15, and AAV16, wherein AAV5 is originally isolated from humans (Bantel-Schaal, and H. zur Hausen. Virology, 1984. 134: 52-63), while AAV1-4 and AAV6 are all found in the study of adenovirus (Ursula Bantel-Schaal, Hajo Delius and Harald zur Hausen. J. Viral., 1999. 73: 939-947).

AAV vectors may be prepared using any convenient methods. Adeno-associated viruses of any serotype are suitable (See, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore. M E Bloom, R M Linden, C R Parrish, Eds.) p 5-14, Rudder Arnold, London, U K (2006); and D E Bowles, J E Rabinowitz, R J Samulski “The Genus Dependovirus” (J R Kerr, SF Cotmore. ME Bloom, R M Linden, C R Parrish, Eds.) p 15-23, Rudder Arnold, London, UK (2006), the disclosures of which are hereby incorporated by reference herein in their entireties). Methods for purifying for vectors may be found in, for example, U.S. Pat. Nos. 6,566,118, 6,989,264, and 6,995,006 and W0/1999/011764 titled “Methods for Generating High Titer Helper-free Preparation of Recombinant AAV Vectors”, the disclosures of which are herein incorporated by reference in their entirety. Preparation of hybrid vectors is described in, for example, PCT Application No. PCTIUS2005/027091, the disclosure of which is herein incorporated by reference in its entirety. The use of viral vectors derived from the AAVs for transferring genes in vitro and in vivo has been described (See e.g., International Patent Application Publication Nos: 91/18088 and WO 93/09239; U.S. Pat. Nos. 4,797,368, 6,596,535, and 5,139,941; and European Patent No: 0488528, all of which are herein incorporated by reference in their entirety). These publications describe various AAV-derived constructs in which the rep or cap genes are deleted and replaced by a gene of interest, and the use of these constructs for transferring the gene of interest in vitro (into cultured cells) or in vivo (directly into an organism). The replication defective recombinant AAVs according to the disclosure can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In some embodiments, the vector(s) for use in the methods of the disclosure are encapsulated into a virus particle (e.g., AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, and AAV16). Accordingly, the disclosure includes a recombinant virus particle (recombinant because it contains a recombinant polynucleotide) comprising any of the vectors described herein. Methods of producing such particles are described e.g. in U.S. Pat. No. 6,596,535.

When the biological sample is transfected with a recombinant vector including the sensor RNA, the sensor RNA is operably linked to a promoter. Suitable promoters of the present disclosure include, without limitation, a SFFV promoter, a hEF1a, a CMV promoter or a variant thereof, an inducible promoter, a CMV-tetO promoter, a tissue or cell specific promoter, etc. When the sensor is operably linked to a promoter, the promoter may be preceded by a 5′ UTR and the sensor RNA sequence may be followed by a 3′ UTR. In some embodiments, the 5′ and 3′ UTR are mmPeg10 UTRs. mmPeg10 UTRs have been described in the art by, for example, Segel et. al. Science. 2021 Aug. 20; 373(6557):882-889 which is specifically incorporated by reference herein. Additional 3′ and 5′ UTRs find use in the present disclosure and have been described in, for example, International Patent Application WO2021055855A1 which is specifically incorporated by reference herein. Sensors that are preceded and followed by specific UTRs have certain benefits over those that don't, for example by increasing expression levels, altering localization, or altering splicing patterns in a way that retains the editable codon in the correct frame (in cases when the promoter causes splicing patterns removing the editable codon from the output reading frame).

In some embodiments, the 3′ UTR and the 5′ UTR are selected from the group consisting of: a Hs Peg10 3′ and 5′ UTR, a mmPeg10 3′ and 5′ UTR, a HsPNMA1 3′ and 5′ UTR, a mmPNMA1 3′ and 5′ UTR, a HsPNMA3 3′ and 5′ UTR, a mmPNMA3 3′ and 5′ UTR, a HsMAOP1 3′ and 5′ UTR, a mmMAOP1 3′ and 5′ UTR, a HsPNMA5 3′ and 5′ UTR, a mmPNMA5 3′ and 5′ UTR, a HsRTL1 3′ and 5′ UTR, a mmRTL1 3′ and 5′ UTR, a HsZCCHC12 3′ and 5′ UTR, a mmZCCHC12 3′ and 5′ UTR, a HsASPRV1 3′ and 5′ UTR, a mmADPRV1 3′ and 5′ UTR, a HsARC1 3′ and 5′ UTR, and a mmARC1 3′ and 5′ UTR. The term “a portion of a nucleic acid” generally refers to a truncation of the nucleic acid sequence. The truncation may be a range of different truncations from the 3′end, the 5′ end, or the 3′ and the 5′ end. For instance, the truncation may be a 1-5, 1-10, 1-50, 1-100, 1-200, 1-300, 1-400, 1-500, 1-600, 1-1000, 1-1100, 1-1200, 1-1300, 1-1400, 1-1500, 1-1600, 1-1700, 1-1800, 1-1900, 1-2000, 10-50, 10-100, 10-200, 10-300, 10-400, 10-500, 10-600, 10-1000, 10-1100, 10-1200, 10-1300, 10-1400, 10-1500, 10-1600, 10-1700, 10-1800, 10-1900, 10-2000, 50-100, 50-200, 50-300, 50-400, 50-500, 50-600, 50-1000, 50-1100, 50-1200, 50-1300, 50-1400, 50-1500, 50-1600, 50-1700, 50-1800, 50-1900, 50-2000, 100-200, 100-300, 100-400, 100-500, 100-600, 100-1000, 100-1100, 100-1200, 100-1300, 100-1400, 100-1500, 100-1600, 100-1700, 100-1800, 100-1900, 100-2000, 200-300, 200-400, 200-500, 200-600, 200-1000, 200-1100, 200-1200, 200-1300, 200-1400, 200-1500, 200-1600, 200-1700, 200-1800, 200-1900, 200-2000, 500-600, 500-1000, 500-1100, 500-1200, 500-1300, 500-1400, 500-1500, 500-1600, 500-1700, 500-1800, 500-1900, or 500-2000 nucleotide truncation of the nucleic acid sequence from the 3′ end, the 5′ end or the 3′ and 5′ end.

In some embodiments, the 5′ UTR and the 3′ UTR are a HsPeg10 5′ UTR and a HsPeg10 3′ UTR. In some embodiments, the HsPeg10 5′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 14)
CTCCTCGGTGCAACCTATATAAGGCTCACAGTCTGCGCTCCTGGTACACGCGCTTCAAC
TTCGGTTGGTGTGTGTCGAAGAAACCTGACTGCGCCCTGAGGAGAACAGCGGAGAAGG
TCCACCGAGCCTGGCGAAAGGTCCGCTGAGCGGGCTGTCGTCCGGAGCCACTCCGGGC
TGCGGAGCACCCAGTGGAGACCGCGCCTGGCTCAGGTGTGGGACCCCATCCTTCCTGT
CTTCGCAGAGGAGTCCTCGCGTGAAATAAGCGGGTTTTGAAAACAAAAAAAAGAAGG
AGTGGAAGAGGGGGCCAGGATCCAGGCCTCCATCCCCACAGAAGTGAAGCTACAGCT
GGGAGGTCTCCTCCCACCCCAACCGTCACCCTGGGTCCCGACTGCCCACCTCCTCCTCC
TCCCCCTCCCCCCAACAACAACAACAACAACAACTCCAAGCACACCGGCCATAAGAGT
GCGTGTGTCCCCAAC.

In some embodiments, the HsPeg10 3′ UTR is according to all or a portion of a nucleic acid at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 15)
ATACCTGTCATGTCCTTCAGGATCTCTGCCCTCAAAATTTATTCCTGTTCAGCTTCTCAA
TCAGTGACTGTGTGCTAAATTTTAGGCTACTGTATCTTCAGGCCACCTGAGGCACATCC
TCTCTGAAACGGCTATGGAAGGTTAGGGCCACTCTGGACTGGCACACATCCTAAAGCA
CCAAAAGACCTTCAACATTTTCTGAGAGCAACAGAGTATTTGCCAATAAATGATCTCTC
ATTTTTCCACCTTGACTGCCAATCTAACTAAAATAATTAATAAGTTTACTTTCCAGCCA
GTCCTGGAAGTCTGGGTTTTACCTGCCAAAACCTCCATCACCATCTAAATTATAGGCTG
CCAAATTTGCTGTTTAACATTTACAGAGAAGCTGATACAAACGCAGGAAATGCTGATTT
CTTTATGGAGGGGGAGACGAGGAGGAGGAGGACATGACTTTTCTTGCGGTTTCGGTAC
CCTCTTTTTAAATCACTGGAGGACTGAGGCCTTATTAAGGAAGC.

In some embodiments, the 5′ UTR and the 3′ UTR are a mmPeg10 5′ UTR and a mmPeg10 3′ UTR. In some embodiments, the mmPeg10 5′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 16)
GGATTTGGCGCCCCCCCTCCTAGGATCTCTCTATATAAGGCAAGCAGTCCGGACTCCCG
ATACACGCGTTTCCAACTTGAGTTGGTGTGTGTCGAAGAATCCTGACCAACTACGACCT
GGGGAGAGCAGCCAACCGAGAAGGTCCACCGAGCCTCGCCTAGGTCTGCTGCGCGGGC
TGCGGTCCGGAGCCTTCTCCGGACCGCGGTCACCCAGTGGACCGGGCCGTCGCGGGAC
CCCTCATCCTTCGTGGCATCGCAGAGGAATCCTCGTGTGGAACAGGCGGGTTTTAAGA
ACCAAAAGACGCCAACCACGAGGGTCCCAGGATCCAGGGCTCCCTCCCCAGGGGAGTG
AAGCCCCTCTCACCGCAGCC.

In some embodiments, the mmPeg10 3′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 17)
CATCCTGCAGTGCTGCGTAGAACCTGTCATGTCCTTTGTAGTCTCTGCCCTCAACTTGAT
CCTGTGCAGCTTCTCAATCTATGACTGTGTGGTACTGGACCTTCAGAGGCGCACAGAGC
TCAAGTCAGTTTTCGTCTTGACTGCCACTTTATAAGTTGACAGGCCTGGGTTTTACTTGT
TAAAACCTCTCACCATCTCAATCACAGGCTGCCAAGTGTCTTTACAAAGAAGCTGATAC
AAACACAGGCCATGCTGATTTCTTACAGAGGGAGAGAAGAGGAAGAGAAGAAGAAAG
AGGAGGAAGAGGACATGACTTGCCCATATGCTGGGCACCTTATAAAGGAAGCCAGACT
TTTCGGTGCAGTATGGAAAGGCTTCCGTGATTCTCTTGCTGCACCCCACGAAACTTCAC
CACCTTCAAACTCCATTTTCACGGTTCCGTTAATTTTCAAGGAGCAGCAACTCGACTGG
TTCTCTGCTACATGAAACACCTCAGCTTGAAAAGGAAGTGCTCTCTCAGACTGACTTGT
GAGTGTGCCTTCACATTCTGGTGCAAATCATG.

In some embodiments, the 5′ UTR and the 3′ UTR are a HsPNMA1 5′ UTR and a HsPNMA1 3′ UTR. In some embodiments, the HsPNMA1 5′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 18)
AGCAGTAACGTCGCGGCGGGTTGCGGGTAGGACTGGACGCCAGAGCAGCCGCGCAGC
GCCTGAACCGCTGCGGGCCGCCGCGGCCGCCCCTTCCCACCCTCGCCTCTGCTGTCTCC
AGCCTCGCTTCTCCGACTTTCCTGCTCCTCTGCTGCCTTCGTTTCTGGTCCTCGGCCGTC
CTCGCCGCCCGCCCAGAGGAGTCCCCGCGCCCGCCAAGAAGCCGCTTTCCGCTGGCCC
GCAGCCGCCGCGACTTCGGCACAGTTTCTCCCTCTGGCTAGTCTCCCAAACGGCCGCTC
CTCGCCCGCGGGAAGACCAGGCTGCGACCGCGAACGCCCGATCCTCTCCAGGAGCCGC
AGCGAGCGCCCGGCGGCCACGCCCCGCGACCACACCCCGGCGGCTCTCGGCCCAGCGC
GCCTGCCTTCGCCGCCCGCCGTCGCTCCTCGCCCGCTGCACGACGACGCGACGCCCCTG
CTGCAGGCGGCGGACCCGACCGGACCCAGACCCAGACGCAAGATGGCGACGGCCGCG
TGACTGCCTCAGCGTCCCCGAGCTCGGCTCCGAGTGCACCTACGGACTGACTGTGGGG
GCAGAGAAGGGCGAGATCAGGACTCTGTCTTTGTTAATCGTGACTGCATGAAGGTCGC
CTCCCTCGGGCCTACTTGGTGGGAGTGTCTGGTATTGTTCTAAGGCCAGGAGCACGGTG
AGCCACAGTCTGTTGGTAGAATTTGGCGTCTTGATAGTTGAGAAA.

In some embodiments, the HsPNMA1 3′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 19)
GTGCCAGGAAAGGCAGCTTTAGTGCAGACCTAGATCACAGCTACTTTTCTTGTCCCTGT
GGGGTCTTACAGATGTGTCTCTGAGTAGTAAAGGCTTAGCCTTGTTCTGTTTTGTTGTTT
TTTGGAGGGGAAGGTTAGTCAGGCCTGAGTATTCATGTAACATTCTAAAATTGTGCCAG
CGAGCACCGTGAACGACTGCAATGCAAGCGGGTCTTGCTGGCTAAAATGCCAGGTAAA
GGGTTGGTTGGACACAGCGCTTAGTGCACGCTGTCATCATGGACATCATAATCAGTTGT
GAAAAACACGCGAACCTATGACACTTCTTATTCCACACTGAATGTGAAATTGCATGTTC
AGATGTTTACTACGAGGCCTGGCTCACAGGAAGTGTTCAGTAAAAGTATGCACTGTTA
GATTACTGATAACGCGGATAGATTTTTGTTTACCATAAATTGTTCCAGATTTATATTAAT
GGAAGGAAGTGTGCATTTATTAGCTATTACTCAACTTTACAATGCAAACATCTTATTTC
TCATCTTTAAACATGTCGACCAGTTTAATTGAAAAGTATTCTGAGACTGCAAAATGGGG
TGTTAAAAAATACTGCAGTTACGGAGCTGTGTAAACCAGTTTCTCATTGCATAAGATAC
AGATGTAAATTGCATGGAGAGGTTGATATGCACCTGTACAGTAATTCACTCCCCCATTT
CACATCTTTGTCAGAGAATAGTTCTTGTTCATACTGAGTGTTCTAAATTTGAAGTTATAT
ATACAAATTAAAATATTTTAAAAATTC.

In some embodiments, the 5′ UTR and the 3′ UTR are a mmPNMA1 5′ UTR and a mmPNMA1 3′ UTR. In some embodiments, the mmPNMA1 5′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 20)
AGCTGCACGCCGGCCGTTGCGGGAGCGCGCGGACATAAGCGCTAGGCAGAGCTGCCTC
CAGCCGAGTTTCTCCCCCATCCCGTGCTTCCTGCCGCTTGGGTTCCCGGTGCTCGGAGT
GCCGCCTCTCCCCACGTAGCCAAGCCACGGCCTCGCGTGACCCTCAGCCCGCCGGCGT
GCCGGGGAGAGGTGGGAGGAGCTGCCACGCCCCGCGACCGCACCCAGCCATCTCCCGC
CCGGAGTCATCAGTCTTGAGCCCACCTTGCTCCCTCTCTTTAAGCGGTGACGCCAGACA
CAGCTGCAGCCAGTTCAGCCCTCCCTGCCCGGACCAAAGGGGGCGACATTCGAGTGAT
AGGCGGAGTGTCCCGTGGCTCCCGACCCTGACTTGGGACCCGGAAGGGACAAGAAGG
AACTCTGGTGTCTTTTGGTAATTGTGGTTGCCTGAAGCTTGTCGCCCCCTGGCCTACCG
GGGGAGGAGAGCATGTTGTATTGTCCTAGGCCCTATAGCCTCGTGAGCCAGTCCAAGC
AGTGTCTGTAGAACTGGCACTTTTAATAGTTGGGTGAA.

In some embodiments, the mmPNMA1 3′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 21)
GTCCTAGGACTGGCCAGGCTGCTTTTGATATACCTGTGACGTTTTAGTAAGCCTGCCAC
TCAAAGCAAGCACCCAGCCTCCTCCTGGCTTGGTTTTGTTTCCTGAGGGATGGGATGGG
GAAGTCAGGCCCGCATATTCCTGTAACATTCACAGAAGTGTGTGATTGTCAGGAGCTG
CAACGGTAAAGGTGGTCCTTACTGCCTAAAGTGAATGCTTGATGCCAGCCCTCCTGGCG
ACATCTCTGACAGCTGTGCACAAACGTGAAGCTGTGACACTTCCTGTCAAGGTGGTTGC
GAAGCTGCAGGTTCTGATGCTAACCGCGCACCAGGTTCACAGACTGTACAGTACAAAC
TCTTGTGTTTGTTACTAACACTGTGGATGGTTTTACTGTAAATTGTTCCCAATTTGTATC
GATGGAAAGAATGTGATTCTATTAGAAGATATTACTCATCTTGAAAAATGCGGATTTCT
AATTCCTCACGGCTCAACGCTGGAATCAGTTTCCCTGAAAAGGATCCTCGGACTAAAA
GTGGCATGTTAGAAAAAACCAGGCCTTTACCGAGCTGCACAATCAGTTCCTCACGGTG
GAAGACAGAAGTCCTAGGGGGAGTGGCCGTGGCCAGTCTACACTTCCCCCACTTCAAA
GACTAGTCATTTAATAATTGGTCAGCGCTAAGGGTTCTAAGTTTGAAGTTATTGAATAG
ATGTATACAAATAAAAATTTGAAAATTCTCAA.

In some embodiments, the 5′ UTR and the 3′ UTR are a HsPNMA3 5′ UTR and a HsPNMA3 3′ UTR. In some embodiments, the HsPNMA3 5′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 22)
GCACTGGCCCACGTGCTGCGCGAGCGAGGGAGAGCCACAGTCTGAGCGAACGTCCGCG
CTGGGAGCCAGGGGTGCCCGACCCCCGTCCGCCGCCGCCGCCGCCGCCGCGCATAGCC
CCCGGAGAGCCCTCTGGGGACCCCGACCAGAAGGGACCTTGCCCTGGGAGAAGGCTGT
GGAGACCTGGGCCTTCTGCGATCACCCTAGGAGTTGATCCAGATATGTGCCTCACGCCC
TGATCACTCCCCCCAAATTAGTATCCGCAGAGATTCGAGGAC.

In some embodiments, the HsPNMA3 3′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 23)
GCTCGGGAGAACAGGGCAACATTTCCTACCACAGGCCAAGGAGACAAAAGAGATATT
GGAAGGAGGGGAAAGAGAAGCCCAGACAAACAGCAGATGAGTTGAGTGGGGCAGAG
GGACAGGGCAGCCAGACCAAGGCCAAGCCTTCTCACCCTTGGCCAGCTGGAAGGGACT
TCAGCAACCAAGACCACCTGGCAACAGGCTCAGTGGGGGTCAGGTCCAGGTCCCCGAA
GAGGTGCTGGAGAGGAAAGCAGGGAGCCACTGCATCCAGCACATGGGGTGCCTGGGC
CTCAGATGGGGACCCCAAAGAAGCAGAAGCTGAAGAAGGTACGGCTGGGGGTTCTGT
CCTGCTCATCCAACCACCCCTAAATACCCACCCTGTGGACTTTGAGCTGAACATGCCCA
CTGGCCCCCAGGCCACATGGGACCTGGAGGAGCCTACCTGGGGCCTGCCCCTGCCAGC
AGGTGCCAGGGCTGGTGAGGAAGAGCTGGGGGGCAGAGGTAAAGCCCTGCAGGGGAG
GCCACAGGGTCCATCCCGTCTTCAGGATCATCTACACTGCACTAGGGGAGCCCCAGGA
AGGCAGCACCCTGGAGGCCCTGTGCCAGTGAGGACAGGAGACCCTAAGGCCCCGGGA
GCCCAGTGCCAGCCAGAGGTTGTGCAGGCAAGGAGACCAAAGATTGATGAGAAGACC
CCCAGCAGGGGTACTGGGTACCCGGCAGGCCAGTGCCCTCACAGTTGACTTGGACCAG
GGTGGCTGTGAAGGGAAGTCTTTGTTGCAAAGGAGGAGGAAAAGGGAGGACTTGGTA
GGGTTTTGTTTCTTCTGCTTGTTTCTGTACAGGGCCACCAGACTCCTGGAGAGATCAAG
CAAGGAGAACCTGGGGCTGCCATGGCCAAAGCAACTCAACAGATGCCAATGCCAATTC
CAAGGCCAGCCACAACCCTGCCACCTTGGGGAATCCAGCCTGGAGGCATCCCCTAAGC
AGCCAGCCATGGCCTGGGTGGAGGCACCTGAAGACGTCTGTCCCAAACTCCCCCAGCC
CTGAGCTGGGAGATGACAGGGGGAAAGAGGCCCTCTCAAGGGTGCCAGATGCCTGGG
TCTCCCAAGAGGGGTCCCCCAACTCACTGTTCCCGGGACAGGCTGCCCCCTGTTCCAGG
AAGCTCATCCTCACCTGTGTAGGCCCCTGTAGTGACCCACGCGTCCAGCAGACGCCCAC
CCACCGCTAGCCGTTGTTCCTGTGCAAAGTAGTGTGCTATGCACCCACCCAGGTGGCCG
CCTCTGGGCCCAAGGCACATGCTGTGAGCTTCCTGTGAGCCCAGGCTCTGCTCACTGCT
GTCCCGCGTCATGAGCACCACCTCTGCTTTCCCTGTGTAGATCTAGGCCAGTGGCTGCT
TGTTCTTGTGGAGCTGTGTGTGTTCTTCTCTGAGCAGCTCCTCCCCGGAGTCCCCCAGCA
CAGTCCCAGGAGATGACAGGAAGGAAGCACCAGGGCAAGGCGGACGCTCACCCTGTG
ACCACGATGGTGACCGTGACTGTGGGAGGAAGAACTGGACCCAGGACGGAGCGGGGC
TGCCCTGCCTGAGGCTCCCGAGGAGCTTTGTGCTTTGGTGTTCCACCCCTGTTGTTACTC
ATGACTCAGTTTCCTTAACCTGGTAGGGTGTTCCCTGCTGTGTTTTCCAGTGTCCTGTGA
CTGTCCTGTGCGGGCCATAGGGCAGGGCCCTGCCCCAGCAGATGGGCTTGGGAGGGGA
CTCCCTAAAGCCAGTGGACACTGCCAGAGTCTACCTTCCTGGCAAGAGGCAGACCCCG
GGGCCCTCAGGAAGGAGGGAGTTGGCAGCGGGGGCTGCAGCAGGAGTAGGAGCAGAT
GAGGCGTCTTGCCAGGAACCTCAGGAGGAGGGGGCCCGGGACCTGTGTGGGACCTGTG
TCCTGTGGTGGCCGTTTGCAGTTTCTCTCTGTGTTGTGATTCCCTTCTCTTCAACGTTTTC
AGTACGTGTTTCTCTTCAATAAACTTCATTCAGTGTTCCA.

In some embodiments, the 5′ UTR and the 3′ UTR are a mmPNMA3 5′ UTR and a mmPNMA3 3′ UTR. In some embodiments, the mmPNMA3 5′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 24)
CTCCCCCCACATTAGAGTCTCTTGAAGTTGGGGCC.

In some embodiments, the mmPNMA3 3′ UTR is according to all or a portion of a nucleic acid having at least 75%, at least 80%, at least 85%, at least 90%, at least 95%, at least 98%, at least 99%, or 100%, nucleotide sequence identity to the following nucleic acid sequence:

(SEQ ID NO: 25)
GCTCCAGGGAACAGGGTAACCTTTGTTCCTGTAAGCCAAGGAGAAAAAAGAGATATTT
GAAGGAGGGGGAAGAACAGCTCAAAAAAATCGCAGAGAACCTTAGAAAGGCACAGG
GGCAATGTAATCAGTCAGAAGTCAAGGCCCCTAAGCCAATACCAGCTGGAGAAAATTC
TAACAAACAAAAATCCATTGCATTCAACTCAATAATGACCACACGCAAGACCTCTAAG
ATTCACCAGAGAAGGAAGCCTCAAGGAACTGCTACAGATGTGTGGGGCCACAAGAACT
CAGATGGGGGTGTCAGCAAAGTGAAGGCAACTTCAAGTCCATATGGGGTGTGGAGGA
GCCTGCCACAGGGTTGTCTTGGGCAGAGGACACCAGTGCCTGGGAACAAGCACGGCTA
GGCAGGGAGACAGTACCCAGGAGAGGAGGCCGCAGGATCCAGCCTGTCTTCAGGATC
ATCTACACAGCTCTAGGGGAGCCCCATGAGGGCAGCACCCTTGAGTCCTTTCGTGAGT
GAACTCCCCCCGCCCCTGTGAAACCTGCAAGCCTCAGGACCCAAAGGACCCGGGAGGC
AAGTGCCAACACAAGGCAGTTTCCTTCTTGACACCCTGACAGGACACTAGTGCCTGAG
AAAAGCCCCAGGCTGGGGCACATGGCACCACAGGGTTAATGCCCTCAGCTGGCCTGAG
AGCCTGGGAGTGTGCAGGACAGGTTTTGTGTGAGCAAACAGGAGCAGCAGAAAGAGG
ACTCTGTGTGTGGGGCGGGGGCAGGCAGGGGGCGGGGGGGGGGGGGAGAGTTTGTTC
TGCTCTCTTTTGTCCAGGGCTGCTGGAGAATTCCAGAGAGCAAGCAGGGACAGCCAGG
TGAATACAACTCTGCCAGGCAGCTGCCAAGGCTGGCCCCAAACCCTGCCGACCTGGAG
GGTGGCCTGGCAGCTTTGCCGAGGTGCCATCCTCCATCTGCCACCCATAACCAAACAG
GTGGACATCACAGAAAAGGCCATCTCAAGGGTTCTGCCAACTGCCTGGCTCTCCCAAC
ACAAATGCCCCTTCCTCTTCCCTTTCCCCTCTCCCCCTTCCCCTGCCACTGCTGTCCAGA
GGCAGGCTGCCCCCTGCTCTGGAACCCCGCCCCCTTATCTGTTAACCTCATAGTGACCC
AGCTGTCCAGTGAGTCCCTTCCCCCATCACGTGCTTACCCACCATCAGCTGCTCCTGTA
TAAAGCTGTGAGTGAGGGCAACTGGAGGCCCTGACTACCTACCTGGTCAGCCACTTCT
ACACCCTGGCACGTGTTGTGAGCTTCTGTGCCAGCCTAGGCTATGCTAGCTGCTCTCTT
ACCCACCTGGTGTTGTGAACTCCTTGTCAGCTTTCCTAGTGTACATCTAGGCCAGCTGC
CTGCTGCTTGTTCTCTTGCAGATGTGTGTGTGTGCGCGTGTGTTCTAACCTGATCAGATG
CTCCCCGGAGTTCCTGAGCTCCGTCTGGAAGCCAGCAGGAACCATGGATGTTGACAAG
AGACTGGCTGATGCTCATCTTACGATTGATTTCAGTCTGGGACAGGAAGACCTGAACTG
GGCATGGGTAGGGCCTGGGGACTGGGCTGTGCTGATCCCCTCCTGTTGTTCCTTGTGAC
TATCATTCCTTGGGTTGGTGGGGTATTCCTTGCTTTGCTTTGTAGTTTCTTGTTACTATCC
TGTGCCAGAATAGGGCAAGGCCACCCTACAGTGGTAATCCCTTGTGAGAGTTCCTAAA
AGAGTGAGGAAATTTTAAGAGGTAGCTCTTGGGCCTTTGGGAAGGGAAAAATAGCAGA
GGGATTCCATGGTGGTGGGCAGACAAGGCCCATCTGCGAAGCTCCAGGAGGGTGGCCC
AGAACCTGGTGCTTCCAGTGGCTGTGGAGAGGTCCTGTGCTGCTAACCCCTTCTCTTCA
ATAGTTTCAGCAAGTGTTTCTGTTCAATAAACTTCATAAATGTTCC.

In some embodiments, the 5′ UTR and the 3′ UTR are a HsMAOP1 5′ UTR and a HsMAOP1 3′ UTR. In some embodiments, the HsMAOP1 5′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 26)
GCGCAGCTTCCCCGAGCGAGACCAAAACAGGTGGAATCCGGGCTGGAGCCGGAGCTCC
GGCGGCGCGGGTGGCGGCACGTCCCTCCAGACAGTACCACAGGCACCTGGAGTACCGG
CATCGGTCGCTGTGGCCCCCGAGTGTCCGTCAGAGCCTAGGGGAGCCTGCCCTCCCGC
GCCTCGTCGGGGCCCGGCCAGGCACCTTGGCCGCCGGCGCACGGACGCGGGCACGAGC
ACTAGATCACGGCTGCTGGACCTCGGCACGTTGACAAGATTTCTCTGGGGTACCGCGG
AGGATTACTTTGAATTTCGGTGGTCGCCTGTGGTCTGGCATATTTAGAACTTAAGTCTA
TTATTTCGGGCACC.

In some embodiments, the HsMAOP1 3′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 27)
GGCAGCTGAGGAGGAGGAGGCCCTTCTCCAGGCAATATTGGAAGGTAATTTCACCTGA
GTCTCAGGGAACCACGAAGGGATATGGCAATGAGTAGAGCATGAAGGTAGAACAGTC
TATATACTCTTGTGACACATACAATCCCTACCTTGTGCTGCCAAGTAACTCATTTTTGTG
CAATTCTCAGTATAAGCCCTTTGTCGTTTCTGTGCCTATTTAAAGTCTCCTAAAGGTGTA
ATTGACTAGGAAGGATGTAGTTCTACACTGCCATTTACCTATTTAAATTCATCCTTGTG
AATATCTTTGTTGTTGTTGTTGAGACAGAGTCTCGCTCTGTCACCCAGGCTGGAGTGCA
GTGGCGTGATCTTGGCTCACTGCATCCTCCGCCTTCCAGGTTTAAGCTATTCTCCTGCCT
CAGTTGCCCGAGTAGCTGGGACTACAGGCATGTGCCACCACGCCCAGCTAAGTTTTGC
ATTATCAGTAGAGACGGGGTTTCACCATGTTGGCCAGGCTAGTTTCGAACTCCTGACCT
CAAGTGATCCACCTGCTTCGGCCTCTGAAAGTGCTGGGATTACAGGCGTGAGACACTG
CGCCCAGCCTCATCCCTGTGAATTTCTTATTGTACACAAGTTCTTTCACTATCTGTGTGC
AGTGCTCTGAGGGGACAGACAAGGCTTGGGTGTATATGCCAACCAGATCTCATTGAAG
TATCAGCTTGTTTGGTACTAGGTGCAAGTGTAGCATGTCACATGTGACCATGTTGGATC
ATTGACAATTTTTAGGTATGTACTGACCTACATTTATGATGAAGATCCTGAGCGGAGGT
TAAGATATTAAGTTATTTTCCATATGAATCAGAATTATATTGATTCTGTGCAATCAAAA
CAAAAGGCAGAATAGAATGCTGAGATTGGTTAAGTTTGCAATGACCATCTTGAACCAC
AGATTTCTGCTATGTGTCATCAAAACATCTAGTTCTGAGTAACATTTTCACGATTGTTAT
AAAATTATAGGTGTGAACTTCTAAAATAAAGGAATGCTAATAAAA.

In some embodiments, the 5′ UTR and the 3′ UTR are a mmMAOP1 5′ UTR and a mmMAOP1 3′ UTR. In some embodiments, the mmMAOP1 5′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 28)
GCTTCGCGCCGCCTCCAGAGGCGGTTGTCTGTTCCCTGAGGCGTTTGCTGCGGTCGTTC
TTCTGCCCTCGGCCGGGAGGCGGGCGCGGGGAGCCCGGGTCTCTCCTAAACCCCGCAA
AGGTCAGACGTCCTCTCCGGGACCCAAGCGATGTATCTACGGGCAGGCTCCCGGACCT
CTGCGTGTTAAAGAGACGAGCACGCACATCACTGTAAGCGGCGGCGGCGGCGGCGGC
GCCCTGGTCGAATTAGAATTTAAATACTCTGAGCACC.

In some embodiments, the mmMAOP1 3′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 29)
CCCAAGACCCAAGGCTGTCATGAGCAGAACAAGATGGAAGAGCTCATTGCAGAGATC
AGCCCATCCACTCTTTATGCTGCCAAAGTAACTCGCCCTTTGTCGTGTTGTTTACTGAAT
GTCTCCCCAATGTTTTACTGATAAAAATGATGTCATTCTACACTGATAATTATTTAAATC
AAAAGACCATTCTGCTAGTACAGTGTCCCAGCAAGTCAAAAGTTTATATGCATGTTTAA
TAGTCATATGTGATGGTGCTATCAGTTATCAATAGTTTTCAGGTGTTGCCCACATTTATG
CTGAAGACCCTGAGCCAAGGCTGGTTAAGATATTTAGTTTCTTTTATTAAAATCAGTTA
TCGATTCTGTGCAATCAAAAAGCACAACTGAATGCTGTGGTCAGTTCTACAACTAGTTT
TAGGTTTGTGACTATCTGATATGTTGCCAAAACGTCTGCTTCTAAATATCATTTTCTTGG
AGTAATAAAGAAATGATAATAAAATATTTGATCTTTGTATACATTGTAATACTACATTT
GAAGGAAAATAATTTTTTGGCTTTGTTTTTAAGACAGTTTCTCTGTGCAGCTTTAGCTGT
GCTGGAACCCACTCTATAGACCCAGGGTAGTCTCAAACTCAGAAATGTGCCTCCTGAG
TGCTGGAATTAAAGGCCTGCACAACTACACTAGGCTGAACAAAAGTTTTTATAGTGTG
AATCTGAAAGCCACTGAAATTACAGACTTAGTTCTTTGAACTCTTGGCACCTAGGGGAT
AGGAACTTGCTTGTTTGACAGGAACCAGACTATAAACAGATTGTGGCGAGTAGTAGGC
CCATCAATATTGAATTTTGAGGGAGAAAGGGAAGATGAAGGTTTGTGTTTTTCCAACCC
TGGTGCTACACACCTATAACCCCAGTGCTTAACAAGTAAAGCCAAAGTCAGGAAGACT
GGCCTCTCAAGGCCAGCCAGAACTACAGAGGGAGGCCCTACCTCCAAAGAGAAAACT
ACATACAGGGAAACTAAGATCACCTGGGAAATGGATGTATGCCCAAGACGAAGCAGG
CAGTAATGAGAGCTGTTCTGGTTCTTGCAAAGCTGCTGTTCTCTCTACTAAGGGAGGGA
ACTACACAAAGAATCAAGGCCAGACTGTCACCGTGTGTACATGTAGTCTTGGGAGTGT
GATAGATTACTCTGAGTTAGGAATACATGGAAAGTTTCCTTATGTTCTCCACTGCAGAA
TGGCATGTGGACTCTTGAGAGCTGTGATTCTGCTCATGTTTGTATAGTGGCTTAAGATA
CATTATGCACTGTCCTGCAGGTCACTCATTTGCTGCCTCGGTGAGAAACTTTACATTTTG
TATAGACACACAAGCATTAATAAATGCCACGCCTCTTCTGTATGTGAGACCCTAAGCTG
AGGGGGTAAACTCGACAGGATAATCCCTGCTTTGAAGGTTTATTCAGACTCAGTATTAA
TGGCTGACATTGGATGTATCCTTGGCCAGCGACTAAGTACTAGCTGAAGAAGAAGCTG
TCTCTTCCCACCTCCCATTTTATGAATAAGATGAGGCACAGAAAAATAACTTGTCTGTG
GTTTTACAGGTAGTAATGTGGCTAGCCCCTGGCTCCAGGTCTTTTTATTCTGGGTAGAT
CATAGGCTCAGAGATCTTTAGGTGCTGGTCTTTGACGCATATATATATATATATATCCC
ATAACATTTGGCAGTGGTGGCAACAGACAGGTGGAGAAGAGTGCCACCTAGTGTTATA
GCTCTAGGGGCCTAGGATGCTAGACCCTTAATTAAGCATTATAGATCTTGTGGAAGCCT
CTAGTAACCCCACCCTTTGCTCTAAAATACTAAGTTATTGGGTAGACCTCATTACAAGT
ATTATAGCTGTTGTATAGACTTGGGAAGTTATTATTCCTTCGCCTTGCCAGTGTTAAATC
GTACAGGTCTCGGGGTGTCTGAGACTTGTAAAAATCACAGAAGCTCTCTGAAACCTCC
AGTGACAAGGGCCTGCTGGTTTTCATTTCCAGCTCTTTACAGCCTTCTCTCCTTTATCTC
TGCCTCTTCGGTTGTCACAACAGCCAGCCACACAGCCATTTTCCAGCTCTGCCTGCCAC
TCCACTGTCTGCTGTTGCTGCCACTCTCCTGGCTTTATATTGCCATCTTAGCCTCACAAC
TAAGAAGTAAGCTAGAAATAGGAAAGGAGGGCTGGAGAGGTGGCTCCTCGGGTAAGA
GCACCGACTGCTCTTCCAGAGGTCCTGAGTTCAAATCCCAGCAACCACATGGTGGCTCA
CAACCATCTGTAATGGGATCTGATGCTGCCTTCTGGTGTGACTGAAAACAGTTACAGTG
TACTCATAAATAAAATAAATCTTTTTAAAAAATGGCAAGGAAACACT.

In some embodiments, the 5′ UTR and the 3′ UTR are a HsPNMA5 5′ UTR and a HsPNMA5 3′ UTR. In some embodiments, the HsPNMA5 5′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 30)
TCTCATTGCATTGCGCAGAGCTCAGCCATCTTTCTCATCCGTCAGGCATTCCTGAGAAA
GAGGGCCACTCTACCTCTCTTGCTGCCAGCCTTCACTCCAGCAAGGAGGGTGCTGGGTG
ACCTGAGCCCACATAGCCCCGAGTGAGCAGTGAGGCTGTCTCCTGCCCTCTTCTGCCTG
GAGGGCTCGTCAGTGTCCCCAGGTGTCAGGCCCTGCCTCCTGACGTTGGCCTTTTCACT
ACAGGCACCCGAAGCACACACAGCAGAGTCCTCTGGACTTTGAGGAAGAACCCACAGC
AGGAGGAAGTCAGCAGGGAGTGGCTGTGTGAAACCTGGGACCACTTCTGCCTTCCTAC
GTGGCAGTGGCTCAGAGTTATTTGAGTGCTGTCAAACTGAGCTGATTGCTGCCCTAGTA
TTAGATCAGTCCATAGAAAGTGGGAGCA.

In some embodiments, the HsPNMA5 3′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 31)
GCTCAGAAGACCGAAGCACTCCTTCCCCTGGCAGGCAGCAGGGACATTTGAACAAGGG
GAAGGGGCAGCTTACACTAACAGGGAACTTGACTGGGGCAGAGGGACAGGGAAGCCA
GGTCGAGATCAAGCCCCCTTACTTTCCACCAGCTGGAAGGGACTTCACCAACCAAGAC
CAAATAGCATCTGGTTCAGTGGGGGCCGGGCCCATAGCCCCTAACAGGTACAGGAGAC
ACAAGCGGGGAACCACTACACTTAGCACATGGGGTGCCTGGGCTTCAGATGGGGACCC
CAGCGAAGCAGGGGCTGAAGAAGGTGTGGCTGGGGGCTCTGGCCTGGTCCTCTGGACA
CCCCCAAATATCTACCCTGTGGACTTTGAGCTGAGCATGCCCACTGGCACCCAGGCCAC
GTGGGACCTGGAGGAGCCTGCCTGGGGCCTGCCTCTGCCAGAAGGAGCCAGGGCTGCT
GAGGAAGAGCTGTGGGGCAGAAGTGAAGCCCCGCAGGGGAGGCCACAGGGTCCAGCC
TGTCTTTAGGATCATCTACACTGCATGAGGGGAGCCCCAGGAAGGCAGCATTCATAAG
CCCCTGCACCAGTGAGGAAGAGAAAGAGGAGGAGAATTAAGAAGAGGATGATCGGGA
GGAGGAAGAGGAGAATAAGGAGGAGCAGGAGGAAGAGGGGGAGGAGAAGGAGGAA
GAGGGGGAGGAGGAAGAGGGGAAGGAGGAGGAAGAAGTGTAAGAAGAGAAGAGAG
GATGTGTTGGGGGGGGTAGTTGTTTTTGGTTTGCGTCATCATCAGGCCCGGGCTCTGC
TCACTGCTGTTCCGTGTCAAGAACTCCACCTCTGCTTTGCCGGTGTAGATCTCGGCCAG
TGGCTGCTTGTTCTCTTGGGGATGTGTGTGTTTTTATCTGAGCAGCTCCTTTCCAGAGTA
CCCCAGCGCAGTTCCAGGAGACGGTGGGAAGGAGGCATGGCAGACGCTCACCTTGTGA
CTGTGACCTCGACTGCAGGAGGACCAACCAGACACGGGAAGGAGCAGGGCTGCCTGA
GGCTCCCCAGGAAGCTTTGTGCTTTGGCGTTCCACCCCTGCTGTTACTCGTGACTCAGTT
TCCTCAACTTGGTGGGGTGTTCCCTGCTATGTTTTCCAGCATCCTGTGACTGTCCTGTGC
AGTCTATAGGGCAGGGCCCTGCCCCAGCGGGTGGGCTCAGGAGGGGGTTCCCTGAAGC
GAGTACACATTGCCAGAGCCCACCATCCTGGCAAGAGGTGGATCCTGGGGCCCTATGG
AAGGAGGGATGTGGTGGGGGGGGCCACACTGAGGGAGGGGCCGGGGACTTGTGACCT
GTAGTGGCTGTTTGCAGTTTCTCTCTGTGTTAGATTCCCTTTTCTTCAGCAGTTTCAGTT
CATGTTTCTCTTCAGTAAATTTTGTTCAGTGTTCCAAAAA.

In some embodiments, the 5′ UTR and the 3′ UTR are a mmPNMA5 5′ UTR and a mmPNMA5 3′ UTR. In some embodiments, the mmPNMA5 5′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 32)
GTTAGGTCTGCTGATAGAGGGAGGGAACA.

In some embodiments, the mmPNMA5 3′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 33)
ACTCCCCCCGCTCCTGTGAAACCTGCAAGCCTCAGGACCCAAAGGACCCGGGAGATGT
GTGTGCTAACCTGAGTTCCTGAGCCTGGTTCTGGAACCATGGATGTTGACAAGGCTGAT
GACTTCAACTTGGGACTATGTGTGATATTCCTCTCCTGTTATTACTTATAATTTTACTTT
CTAGGGTTGGTGGGGTGTTGCATGCTATGGTTCCACCAATTCCCTGTGCCTGGACTGTG
TATGCTATAAGGGAGAACTACACTGTAGTGAGTGATCTCTTGAGATAGCAGAAGGCCC
TGTGTGAGCAGAGCCTATGGCTTCTAGTGACCACTCAAAGTTTCTGTTATGTTTTTAAT
GGTTTTAATACAATTTTTGTTGAATAAATCTCATGAATGTTCTGGCTAAAA.

In some embodiments, the 5′ UTR and the 3′ UTR are a HsRTL1 5′ UTR and a HsRTL1 3′ UTR. In some embodiments, the HsRTL1 5′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 34)
ACACCCCCCCAGCACTCGGCACAGCCTCGCTGAGCAGCCTTCAGCAGCAGGGTGTGGT
GGGGAGCCTCGGAGAGTCTGGGGTTCCATCCTGGCCCAGCCTCACACCAGCTGAGAGA
CGACCGACTGAGCTTCGAGGACAGGAAGCCACCGGCATCACTAAGCCACCGGCATCAC
TTCATCCCCAGCCTCACGCTTGGGACTGGGCCCGGGGAAGCAAGCAGCGAGGATCGAG
TGGCACAGGACGGGAGGAGATCCACTTGAACGCTTGCAGCCAAGGTTCTGATCTCCAC
GGTCCCAGCTACTGACTGGACGCCATCACAACCTTACCAATCTTCAGAATACACTCCTT
TCCATCCGACGAA.

In some embodiments, the HsRTL1 3′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 35)
AGACGCCTCCCTGTCTCCAGCAAAGAGTCCCCTCTATTTCTCACCCCAACACCTCCCGA
CGTCCCCTGGCCTCCCCGCGCCTCAGCCTGCTTCGCTTCCCTTCCTGCCACTCCTGACCC
AAGAGGAACCCACTTGTGAAGGGCAACAACTTCTTCAACTCATCAACCAGCAATGCCA
CCTGTGCTAACGGACTACCCAGTCCTCAGAGACCCAGCGCCAGCCCTGGGCTACGAGT
TCTCCGTTCTCCAGGTCCCAAGCCCATGCCCGGGAGTGACATCCATGAGGGAGAAAGC
AGTGGTGAGCAGGAAGTGACCCCATGACGCGTGGCCAGATGAAGAAGCAGGCACAGC
AGGCGCAGCCAAGCAAGCAGGTGACGGGGCCCCCCACCCGGCCTGGTGATGGCCACCC
TGGGACACTGACCTCGGGTGCTCCACTGACTTCTTCTACCTCCACTGCCGGTGCTCCTG
CTCCCCTGAGCTCCCGCCCCCGCCCCCCAACCCCCATCCCCCAACCTCCCGACATCTGA
TGATGTGGTGCCTCCCTCACCCCCATCACACCCTGCAAGCAAACCCACCCCCACCCCCA
CCACATCTGCTTCAGCTTTTTTGACCCCGCCTTCCTCTTGCCCTGAGCTCTGGACTTACC
TGGACAAAGCCGCTCTTTGGGGCATCCGGACGTACCAGTGCCACTTACCAGACTTGCA
CAGCAAAGAGGGCCCCTGCATGCCTTGGGGCGCCTTTCCCTCCCAACACTGAACACTG
GACAGTGCAGGGGTGCAGGGGCCCCTCATACAGGGATGGAGTCCCAGAAGGCACACA
GACACCCAGGGGACCATGCCGAGTCACAGAGAGGCTCTGGCCACACCACCGCTTTGCA
AGGGGAGTAGACCCTTCCCCACCCCCCGAGCTTTCTCACCCCCCAATCTTTTTGCACTC
CTCAAATAAAGCAAACTAAAAGAC.

In some embodiments, the 5′ UTR and the 3′ UTR are a mmRTL1 5′ UTR and a mmRTL1 3′ UTR. In some embodiments, the mmRTL1 5′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 36)
GCTCAGAGGCAATCAAGGAGCTAACGTGACCAAGTCTCGCTCTCGGGCAGGCGCTAAC
AGTGGTTTTGGTCTCCACAGTTGCGGTCTCTGACCACACACATCACAATCTTACCAGTC
TTCAGAGCACACTCCTTTCCAACCGACGAC.

In some embodiments, the mmRTL1 3′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 37)
AAACACACCAAGTCACTGTCATTTTCCCTTGTGCCTCGGCCAGCTTCGCTCAGATCCAG
CACACCCTCTTGCTACTCCTAACCCAAGGGGAACCCACTTGTGAAGGGCCACAACTTCA
TCAACCAGCAACATCATCTGTGCTAAAGAACTAGCCAACTATCAGCGTCCCAGAACCA
GCCAAGACTGTGTAACCCTAACTGTCCTAACTGTCCGGGCCCCAGGCCCACAAAAGGG
AGTGAGACCAATGAGGAGAGAGCAGTGATGAGCAACAGTGACCCCGTCACCCCAGGC
CAGATGCAGAGCAGATACAGCAGGTGCAGCCAAGCAAGCAGATGCCAGTGACCCCAC
CACCACCCCCTGAGTGGCAGGCGGCCACCTGTGCATCCTGGGACACTGACCTTGGGTG
CTCCACGGACTTCTACCTCTACACCGGTGCTCCGACTCCCCTGGACACGCCCCTACTCC
CTCCCAGTCTCCCAAGGTGCTGTGGTTCCATGCTTTCCACAACCCCAGCCCACCCTGCA
AGCAAGTCCACCCCCCCCCCCACAACCCACCCACCCACTACCGGTCTGGTCCGGCTCCA
TTGACACCTCATCTGACCCTGAACTCTGATCTTACCTGGGCAGAACTGCTCCAAGGAGC
ATTCGACGTACCAGTGTGACTTACCAGATGGACTGATCCACCCTGAAGGTCCCTGAGA
CTCAGAGGACCCAAGAAAGGGTGGTAGCAACCAAGTGGGGCTCCCTTTTCCCTCCCCA
AATGGACATATGGACAGTGCAGGGGACAGAGACCTTCCATAAAGGAGGGAGCCCCCA
AGAGACACTGGTGAAGCCCCAGATACCCAGAGTACTGTGCCAAGGAGCCAAGAGTGCT
CTGACCAGACCCACTGCTCACTGCCACAGGAAGGAGAGGTCCTTCCATTGCTTTCCCAA
CTCATACCTGCTTTTGCCCCATGAATAAAGAGAAGAAAAGATTA.

In some embodiments, the 5′ UTR and the 3′ UTR are a HsZCCHC12 5′ UTR and a HsZCCHC12 3′ UTR. In some embodiments, the HsZCCHC12 5′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 38)
GGCGCTGCCTCGTCTCTGCTACCCCTGGTTGGGCGGCCCTGCGAAGCAGCTCCTTCGGG
CAGCCCCGGGTCGCTTAGCGGCCAAGGAGGCTTCAGTTCTTTGCCGCCTGCAAGGCGG
AGACCAGAAGGCGGAATCCACAGCTGGCGACGCGGGAGCATCTGCTGTCCACCAGCG
GAGCACAGGCCATCAAAGCCGCATCTGAACTTGAATTCTGTGCAGCTGATTGCAGAGC
TGGACCCGGATCTGCGACCCCCTGTGGACAGAGGTTGACCGTACCCCGGAGAGGAGCT
TTCTCACGGAGGGCACTGGTTGCAGAGGCTGGAAGTGAAATAAAGACGCGCTCTTGTT
TCAGAGTTCGTCCCCTGCTGAGATAGGAAGGCAGAGCCACCTCCTCTCCTCTCCCACCT
GCAGATTAAGCTTTTCTAAAAAGCCTAGGCATCTTCTTATATTCAGATACCCTATCGTC
GTCAGTC.

In some embodiments, the HsZCCHC12 3′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 39)
AGGGACCACCCCCAGGTTTCAGTGAACCCTTACCTATATTCAGCATCCAGTAGTGGGA
AAACTGGGGTGGGGGTGGGGGGGGACTTCTAACTGCATGAATTAATCCACAAAGCGG
CTATCTTTTGGGGTGGAGTAGAAAGGGTCTTGGATACCAGCACATTGGAGGGAGATAG
CCTGACCTCTGTCCTTGCTCCTTCTCCCTGCAGCCTACGGGTCTGTTTTCTGTGTGTGCC
CATTTCCTTGACAGCTTTATTCTTTGTGAAAGTGGTATAATTTATTGTTAAATATTTGAA
CAATAAAAAAGGTACAAAAAGTGAAGTACAAATTACCCAAATCTCTCCACCCTTATAT
AATCATTGTCAACCCTTTGATGAGTGATATTTCCCTATACCTATGTACCCAGATAGATA
TATGCATAGATAAAAGTGATGAAATATAAGTGCTGTTCTATCTGTATTTTTTCACCAAA
CAATATATGTTGTGAGCTTCTATGTCAATAAATATATATATCAGCA.

In some embodiments, the 5′ UTR and the 3′ UTR are a mmZCCHC12 5′ UTR and a mmZCCHC12 3′ UTR. In some embodiments, the mmZCCHC12 5′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 40)
TGATTGGCTCCGCTGGCCAGCTCGTCACACTCTTTTGTGTCAGTAGGCTGCTGATAAAA
GCTTTGCAGCTGCCTTGGAAACTGCGCTATTCGAATCCGGTTACCTGTGGGCAGAATCC
ATAGCTGAAGACACCACAGCAGCCTCTGGCTACCAGTGGATCACAGTAGCAGCCCATC
AAAGCGGAATCTGAACTTGAATTTGGTGCAGCTGGTTGTAGAGCTGGTCTTGAGCATC
AGGATCTGAGACCCCCAGTCGATCGAGGTCCATCAACTGGACCCTGGAGGCAGGCTTT
CTCACGGGAGGGAGGACACAGGTTGCAGAAGCTGGAAATACTCCAGTTTCCCAGCTGA
TCCCCCTACTGAGATTGGAGACTGCTGCACTGCTGAAGATAAAGCGTTTTTGTTTTGTT
TCTAAAACGCCTTGAACTTCGTGTATTAAGATATCCTATCACTGCTGTCAGAC.

In some embodiments, the mmZCCHC12 3′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 41)
CCAGCCTTAAATTAACCCTTACCCGTATTAAATGTTTAACGGTGGAAAAGTTGTGGGGA
CAGATTTCTAATTGCATGAACAAATCCACAAAGTGGCTTTCTTTGGGGGTGGAGAAAG
GGTCCCGAATACCAGTACAGTAAAGAGAGGTGGCCTGACCTATGATCGTGTTCCCATT
CCCCACTGCTTAACAGTCTATTTTCTGTGTGAACCTCTCTCCTTGGTGGCTTTGTTCTCTT
TGTGAAAGTGGTATTGTTCATTGTTCAATCTTCAAGTAAGAAAATTATTAAAAAGTACA
AATTACCCAGACCTCTCTACCTTGTGAAACAATTGTCAGCCCTTTGGTGCCTATCCTTCT
AAATATTTCTCTATATCTGTGTTCCTAGATTAGAAATATGTATAGACGAAAGTGATCAA
ATAGAAGTGTTGTTCTATATGCTGTATTTTTTCACCAAAACGTATGTTGTGGCCTTCTTT
GTCAATAAATATATACATATATGTCAGCATCTACTTT.

In some embodiments, the 5′ UTR and the 3′ UTR are a HsASPRV1 5′ UTR and a HsASPRV1 3′ UTR. In some embodiments, the HsASPRV1 5′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 42)
ACAGCACTGCCTGCCTCTGCCCCAGCAGTCAGCCAGCCGACCGCGCCTGCTCCCTCCTG
CTTGCCCAAGGCCGGGCAAGTCATCCCCACTCTGCTTCGAGAGGCCCCGTTTTCCAGCG
TGATTGCGCCGACACTGCTCTGTGGGTTTCTCTTCTTGGCGTGGGTTGCTGCTGAGGTTC
CAGAGGAGAGCAGCAGG.

In some embodiments, the HsASPRV1 3′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 43)
GAAGCCACCTTTTCTTTAACCTCCTAAATATTGGTGGGAAGACCCACCGCTGTGGGGGG
GGTTGCATATCCTCATGGGGGTCACTGGGCTTGGCCAGTCTGCTTATCAACTCTTGCTC
TTCTCTCCCCTTTGCCTCCCTCTGCAGGGGCCTTAATCTGCCCCTGGTAGGGGAGGCTTC
CACTGAACAGGCACAGGTGAGGGAGAGCAGGCTGGCTTAGAGGGACAGGGTCCCCAT
GGTCATCAAGCTGCTGTTGATGACAAAGACTCAAAGGCTGGAAGAGCTCCCAAGGAAG
CTAGAAATGCTTGTCTTTGAAAGAACTGTGGGACCCCTTCAGATTCCCTGAGGTATGGC
TTGGTCACTCTCAGGTCCTCAAAGCCTGTCTTAGTTGGGCTGGGTCCTAGCTGCAGGGT
CTTTGTGAGGGTCACAGTTGCTCTGGGACACCTCCCTGAAGAGCCTTTCCACCTGTACA
ATCGTATTTTCTTTCTGTCATTTGCTTTGAAGCCCATTGTGCCTTATGCCAATAATTCAA
TTGCTGCAAACACCAATAAAGATTGATTCATGG.

In some embodiments, the 5′ UTR and the 3′ UTR are a mmASPRV1 5′ UTR and a mmASPRV1 3′ UTR. In some embodiments, the HsASPRV1 5′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 44)
ACAGCCTGCCTCTGTGCCCAGCAGCCAGCCAGACACTTTGTACCGGCTCCCTTCAACTC
GTCCAGGCAGGGCAAGAACACGGCCCAGCCGACAGAGCCCTCGCTCTCCAGCGTGATT
GCGCCCACACTCTTCTGTGCGTTTCTTTACTTGGCTTGTGTTACTGCTGAACTTCCAGAG
GTGAGCAGAAGG.

In some embodiments, the mmASPRV1 3′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 45)
GAAACCCCATTTCTTGTTCCCAGCATTGGTAGGGGGACTTTGTGTTGGGGGGAGCAGAT
GTCCTGGGGGGTATCATCCGGCCTAGCCAGTCTTTACACCGGTTCTCAGTTTCCCTCCTT
CTACAGGGGCCTTGCTTTGCCTTTGTTTGGGGAGGGAGGCCAGCTTGGTGGCCTAAAGC
AGTGTCCCCAAGGTCTGCAAAGACTTCCAAGGCTGGCAGGAGCTTCTGAGGAAGCCAG
GAATGTCAATCTTGAGAGAGGACCCTTTTAGATCCCCTGAAGTATGGCTCAGTCACTTT
CACGTCCCCAAGCCTGCTGAGCTGAGCCTGGTCTTGGCTAAGACCCTCACAATCCAGAT
GCTTGGAGGAGACTGGCAGCTGCTCTGGGAGTCCTCCCTGAGTCCTCCCACCTGCACAA
GGATGCTCCCTCCTGTCCTGTCACTTGCCTTGAATCTCATGGAGCCTGTATCAATAATTC
AATTATTTCAAAACACCAATAAAGATCTGTTCATGG.

In some embodiments, the 5′ UTR and the 3′ UTR are a HsARC1 5′ UTR and a HsARC1 3′ UTR. In some embodiments, the HsARC1 5′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 46)
GCAGAGCTCGGGCGCGGCGTCCTCCCTCCGCAGCAGCCGAGCCGGACCTGCCTCCCCG
GGCGTGCTCCGCCGGCCCCGCCGCCGGCCCGCAGCGACAGACAGGCGCTCCCCGCAGC
TCCGCACGGGACCCAGGCCGCCGGACCCCAGCGCCGGACCACCCTCTGTCCGCCCCGA
GGAGTTTGCCGCCTGCCGGAGCACCTGCGCACAG.

In some embodiments, the HsARC1 3′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 47)
AGGGCATCCCGGAGCCCCCAGCCTGCCCACTACATCCAGCCTGTGGCTTTGCCCACCAG
GACTTTTGAGCTGGGGCTGACTCCTGCAGGGGAAGCCCTGGTCCAGCTGGGTGCCCCCT
CGAGCTCCGGGCGGACTCGCACACACTCGTGTCATCCAGATGTGAGCACCGCACCCAG
CGGCAAAGAGCCCTCCCCCCTGCAGGGCTCCACCCATCACCCTCCCTCCGTCTGTCTTT
CCGGCCTGGACCCCACCCTCCACACTCTCAGGCCATCACAGAACACCCCAGCTTCCTCA
TTCTGCTACAACACCCAGGCCCTCTGGACATCCAGAAAACCAAGTGTCCGGATGGCAG
GGGCCAGCGGCCACCAAGCTCATGGGACACCCAGAGCAGAAGCTAGGGCAGAGCCAA
TGCTGAGGGAGCCTCGACTTCCGGCGCCGCCGCCCTCTCCCGGCATCCGCAGAGCCAG
CTGACGCCCTCCCTGCCTCCCAGGGCAGCTGGCCAGCCTCGGGCAGCGCGGCCCCCTCC
TCCCAGGGGAGAGTAGAAGTCGCACACGCAGCAGAGCAGACCTGATGTCCCGGTGCTT
CCTGGCCCCTCAGCTCCAGTGATTCACGCCCGCCTGGAGAAGAATCAGAGCTCAGCTC
ATGACTCACCCATGGCAGGCGGAGGGTCCCAGAGGGGCTGAGTCCTCAAATCCGGCTG
AGGCAGCAGCTGGCACCATCAGAGCCAGGAGAGTGACAACAGGTAACGGAGCACCAC
CCTTCCACCCAGACCCCACCATCAGCTGTCCCGGCCAAATGAGCTCCTCCCCAGACCCC
AGACACCCTGCGGCCCAGGCCACTCCCCAGTGTTCGGGCTCGCTGGGAGGCTCTGACG
GGGTCGGGGGGCCTCTGCCTTGGAAGACCAGCGCCATCCACCGGCCCCAGCCCCTCCC
CATGGAGCCCTGGCTACTGTCCTGCGTGGTCCTTCACTGCCCACTCTCCTGTTCTTTCAG
GTCTCAAGGTTCCCACAAAGTCTTTGCTGCTGTGCTGGGCACCACCCACCCCTCACCTT
GCAGGCTGCCTGCGTGGGAGGCGAAGTCCCAGGACAGCCCAGAGGGGGGCTACAGAG
AGGAGTCGGCTGCAGCAGAGGGCAGGAGCCCCAGCTTAGCCCTGAGCGCCAGCGCGA
GGACCAGGGCCTGCCACTAAGCCCGCCCCGCTGGCCGCCAGCTGCCCGTCCCCAGAGC
CACTGCAGCAGGAGTCGGGCCCTGCCTCCCTCCCAGCAGGGAAACCCCGCCCGCTGCC
AGGCCATCCTCTCTGCCAGAGGCTTTCATGAGCCCCAAGGCTGGGGCCACAGCTCCTAC
CCCTGCCCAGCAGCCCTGAGCTCAGCTGCAGGAAGGACATCCCAGAAGCCATGGCTCC
TGGGGCGCTTCCAGGCATTCTGCCCTGCCCCGACACCAGAACCCTGGTGCTGGTGGGCC
ACTAGCGTCTGCAGCCTAAGCAGGTGCTGGCTCAGGGTTCATCGTTCTGCCTTGTCCAC
TGGGGGACCAGCCCTGCAGACCACTCTGACAAGTCTTCAGCCCACACCCTGCCAGCCC
CACAGATTTTATTTTTGCACATAAGCCATAACCAATCCTCAAGGCTGGCACAGGCTTTG
GGGAAGCCCTGGAGCCTGTGAAGACCCTGGAAACCTCATGAGGCTGTGGCCAACCCCT
GCCCCTTGCCCCACACAGACCAGGCCTTAAATGTCGGTCCAGGCCCTGTGCACCTTACC
CCAGAGACAGACTCTTTTTGTAAGATTTTGTTAATAAAACACTGAAACTTC.

In some embodiments, the 5′ UTR and the 3′ UTR are a mmARC1 5′ UTR and a mmARC1 3′ UTR. In some embodiments, the mmARC1 5′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 48)
GCAGAGCTCAAGCGAGTTCTCCCGCAGCCGCAGTCTCTGGGCCTCTCTAGCTTCAGCGG
CGACGAGCCTGCCACACTCGCTAAGCTCCTCCGGCACCGCACACCTGCCACTGCCGCTG
CAGCCGCCGGCTCTGCTCCCTTCCGGCTTCTGCCTCAGAGGAGTTCTTAGCCTGTTCGG
AGCCGCAGCACCGACGACCAG.

In some embodiments, the mmARC1 3′ UTR is according to all or a portion of a nucleic acid having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% nucleotide sequence identity to the following nucleic acid sequence:

(SEQ ID NO: 49)
AGGGGCCAGCCCAGGGTCCCCAGCCCGCCTGCCACACCCAGCCTGTGGCTTTTGCCAA
CTAGGACTTGAGCTGGGGCTGACTCCCAAAGGGATGCCCTGTCCAGCCAGACATCTAC
TCACCCACTGACCTGGCCTGACTCACAACTGCCACACAACCATGCTACATGGACTGAA
CATCAAGAAGCCCCTTTCCCATAGGGCTCCCACCTGCCGCCTACCCCTCATCTGTCTGC
CCTGGTCCTGGCCCCCAGCCCCATTGGCCTCACCCTCTACACTCTCAGACCATCACAGA
ACACGATCTGGCTTCCTCATTCTGCTCCAGTGTCCAGGGCTCTTTGGGTAATCAAGAAA
CCAAGTGTCTGAAAGGCAACAAAAAGTAGGCACCAAAACCCAGGGGACATCCTAGGG
CAGATGCTAAAGCAGAGATGCTGAGGGAACCTCAACTTCCGGGGATGCAGCCCACTCC
TGCAGACACAGCAGATCCAGCTGGTACCCTAGCTGCCGCCCAGGGCAACCGGCCAGTC
TTGGGCAGCATAGCTCCCCTCCCAGGGGTGAGCTGAAGCCACAAATGCAGCTGAAGCA
GCAGACCTGACATCCTGGCACCTCCTGGCCCCCAGCAGTGATTCATACCAGTGAAGAA
GAGCAGAGCTCAGCTCCGTGACTCAGCCATGGCAGGCGGAGGGTCCCCGAGGGGCTGA
GTCCTCACACCCAGCCGAGGCAGCGGCTGGAGCCTACAGAGCCAGGAGAATGACACC
AGGTCTCAAGGCTGCTGAGAAGTCTTTGCTGCCATGTCTGGAAAGGGCATCACCACAC
CACCAGCACCAGCACCATCCTCTCTTCTCCTGAAGCTGCCTACATGGGTTCCAAGACAC
TTTCAAGGCAGAGAAAACAAGATTACAGAGAGGAGGTGCCTGGCAGGGGGCAGCACC
CCAGCTCAGCCCAAGAGCTGAAGGTGAAGACAAGCCAGCATGAAACCACGGGTCTGC
CATGATGCCCGCCCCGCTGGCCGCTCACTAGCTGCCTGCCATTAGCCTCTGCAGCTTGA
GCAGGGTCTGTACCCTTGGTCCTCTTGGCACAGAGCCCAGCTCGCTGCATGGCCTTTGG
CTCCCCGACCAGACCCTTGCAGGAGCCTTAAGGCTTGGGCCCCTGCCCAGCCTGATCTT
TCCTGCTGTGCCCTGCCTGCCAGGTCAAGCCCAGTCCCAGGAGACCCCAGGCCTTGGCT
CCTAGGCTGTTCCATGAACCTCCCTGACCTGCCTGGTGATTGCCCAGCTGAAACCTCAG
CCAAGCCCCAGCTCCAATTACCTTGTGCTGGTAGCTGCTTGTGTCTGCAGTCTGAGTAG
GCCTTGTTGCGGTTCCTCCATCTGCCTGGTCTATTGGTGTTCTGAGACCAATTCCACTGA
TGTTCTGACAGATCCTCCACCCTGTGCCCCTGCCAGCCCCCACAAGTTTATTTTTGCACA
AAAGCCATGACCCATACTCATTTGGCTGGCATAGGGTGTGTAGGTAGGCCCTGGGGAC
TAGGGAGACCCTGGAGATCTCAAGAGAGTGTGGCTATCCCCTATTTTCACCAAGCCTTG
AATATCCAGCCAGGCTGTCTGCCCATACCATCTTACCTCAAAGACAGATATATATCTAT
ATATGATTTTGTTAATAAAACTATGAAACTTATT.

In some aspects of the present disclosure, the sensor RNA includes one or more MS2 hairpins. In some embodiments, the sensor RNA includes more than one MS2 hairpin. For example, the sensor RNA may include two or more, three or more, four or more, five or more, six or more, seven or more, eight or more, nine or more, ten more, or more than ten. In some aspects, the sensor RNA include one or more TAR RNA elements. For example, the sensor RNA may include two or more, three or more, four or more, five or more, six or more, seven or more, eight or more, nine or more, ten more, or more than ten. In some aspects, the sensor RNA include one or more BoxB stem-loop. For example, the sensor RNA may include two or more, three or more, four or more, five or more, six or more, seven or more, eight or more, nine or more, ten more, or more than ten. In some aspects, the sensor RNA includes MS2 hairpins and BoxB stem loops, MS2 hairpins and TAR RNA elements, or BoxB stem-loops and TAR RNA elements.

In some embodiments, the method of detecting a target RNA further contains combining the biological sample with an ADAR protein or a coding sequence thereof. The ADAR protein may be any ADAR protein from any species. For instance, the ADAR protein may include without limitation, an ADAR (ADAR1), an ADAR p110, an ADAR p150, an ADAR2, an engineered ADAR protein such as a protein containing a deaminase domain of ADAR2 or a variant thereof and a MS2 RNA binding protein (MCP), an engineered ADAR protein that lacks a nuclear localization sequence, an engineered ADAR protein containing a nuclear export sequence, an engineered ADAR protein containing one or more dsRNA binding domains from one or more distinct ADAR proteins, an engineered ADAR protein containing a TAR RNA binding protein, an engineered ADAR protein containing a Lambda N peptide, a split engineered ADAR protein wherein the N and C terminus of the deaminase domain are produced separately and the two halves binding to one another in the presence of the target RNA, etc. Suitable engineered ADAR proteins have been described in Katrekar et al. (Nat Methods. 2019 March; 16(3):239-242.), Biswas et al. (iScience. 2020 Jul. 24; 23(7):101318), Matthews et al. (Nat Struct Mol Biol. 2016 May; 23(5):426-33), Cox et al. (Science. 2017 Nov. 24; 358(6366):1019-1027) or Kuttan et al. (Proc Natl Acad Sci USA. 2012 Nov. 27; 109(48):E3295-304). Split engineered ADAR proteins are described in Katrekar et al. (Elife. 2022 Jan. 19; 11:e75555). When the sensor RNA contains a start codon in place of a stop codon, a particular ADAR protein may be used. In some embodiments, the ADAR protein is ADAR2 when the sensor RNA contains a start codon in place of a stop codon.

In some embodiments, RNA editing proteins other than ADARs are used. For instance, proteins of the apolipoprotein B mRNA editing enzyme, catalytic polypeptide-like (APOBEC) family may be used. Examples of suitable APOBEC proteins include, without limitation, APOBEC1, APOBEC2, APOBEC3A, APOBEC3B, APOBEC3C, APOBEC3D, APOBEC3F, APOBEC3G, APOBEC3H, APOBEC4, etc.

In some embodiments, the sensor RNA further contains a nucleotide sequence containing a cleavage domain followed by a nucleotide sequence encoding any of the ADAR proteins described above wherein the nucleotide sequence containing the cleavage domain is after the nucleotide sequence encoding the output protein. In some embodiments, an ADAR protein is used instead of a marker protein as the first nucleotide sequence.

In some embodiments, the sensor RNA further contains a nucleotide sequence encoding a region that hybridizes to a second target or predetermined RNA wherein the sensor RNA contains a second stop codon wherein the sequences of the first and second target or predetermined RNAs are different. In some embodiments, the stop codon that contains at least 1 base, 2 bases, 3 bases, 4 bases, 5 bases, 6 bases, 7 bases, 8 bases, 9 bases, or 10 bases that is/are mismatched with the second target or predetermined RNA sequence. In some embodiments, the stop codon that contains at least 1 consecutive base, 2 consecutive bases, 3 consecutive bases, 4 consecutive bases, 5 consecutive bases, 6 consecutive bases, 7 consecutive bases, 8 consecutive bases, 9 consecutive bases, or 10 consecutive bases that is/are mismatched with the second target or predetermined RNA sequence. In some embodiments, the sensor RNA further contains a nucleotide sequence encoding a second nucleotide sequence that hybridizes to a second target or predetermined RNA wherein the second nucleotide sequence contains a start codon. In some embodiments, the sensor RNA further contains a nucleotide sequence encoding a second nucleotide sequence that hybridizes to a second target RNA wherein the second nucleotide sequence contains a non-start codon that can be edited to a start codon. In some embodiments, the stop, start or non-start codon can contain at least 1 base that is mismatched with the second target RNA sequence. In some embodiments, the stop, start or non-start codon can be contained within a stem-loop sequence contained in the second nucleotide sequence that hybridizes to a second target or predetermined RNA. In some embodiments, the biological sample is combined with two or more sensor RNAs that detect two or more distinct target RNAs.

In some embodiments, expressing a protein in a target cell further contains combining the biological sample with a protein that specifically localizes the sensor RNA to the location of the target RNA. For examples, a protein that specifically localizes the sensor RNA to the location of the target RNA may be a dCas9 or a dCas13 protein that has a guide RNA directed to the genomic locus corresponding to the target RNA (in the case of dCas9) or the target RNA directly (in the case of dCas13). In some embodiments, the dCas9 or dCas13 is engineered to be linked to a MCP, a TAR RNA binding protein or a Lambda N peptide.

In some embodiments, the methods of the present disclosure can be used to produce a target protein in the absences of a cell. In these embodiments, a cell-free system includes the biological sample, the sensor RNA and the ADAR protein. The biological sample may include any target RNA. For example, the biological sample may be a sample including viral matter such as viral RNA wherein detection of the viral RNA leads to production of the output protein. Suitable cell-free systems include those described by Kuruma et al. (Nat Protoc. 2015 September; 10(9):1328-44) and Lavickova et al. (ACS Synth Biol. 2019 Feb. 15; 8(2):455-462).

Additional embodiments for using the sensor RNAs disclosed herein can be found in, for example, PCT application number US2023/063245 which is specifically incorporated by reference herein.

Methods are provided for generating a pseudouridine-containing sensor RNA, the method including: combining: (i) a first segment comprising: (ia) a first nucleotide sequence comprising a nucleotide sequence encoding a marker protein, and (ib) a second nucleotide sequence comprising a first cleavage domain, wherein the first segment contains one or more pseudouridines; and (ii) a second segment comprising: a third nucleotide sequence comprising a nucleotide sequence that hybridizes to the target RNA and a stem-loop sequence comprising one or more editable codons, wherein the second segment does not comprise a pseudouridine; (iii) a third segment comprising: (iiia) a fourth nucleotide sequence encoding a first cleavage domain, and (iiib) a fifth nucleotide sequence encoding an output protein, wherein the third segment contains one or more pseudouridines.

In some embodiments, the pseudouridine-containing sensor RNAs of the present disclosure do not contain pseudouridines in the sensor nucleotide sequence. Sensor RNAs that do not contain pseudouridines in the sensor nucleotide sequence can provide certain advantages. The incorporation of pseudouridines can reduce the immunogenicity of the sensor RNA, however, the pseudouridines also can increase stop codon readthrough and impair ADAR editing. Sensor RNAs that have pseudouridines in segments that do not have the sensor nucleotide sequence can have reduced immunogenicity but also allow for ADAR editing and the absence of stop codon readthrough.

The methods described herein include combining a first segment, a second segment, and a third segment together to form a single RNA molecule. In some embodiments, the first segment contains a first nucleotide sequence containing a nucleotide sequence encoding a marker protein and a second nucleotide sequence containing a nucleotide sequence encoding a first cleavage domain where the first segment contains one or more pseudouridines. In some embodiments, the first segment has all uridines replaced with pseudouridines. In some embodiments, the second segment contains a third nucleotide sequence containing a nucleotide sequence that hybridizes to the target RNA where the second segment does not contain pseudouridines. In some embodiments, the third segment contains a fourth nucleotide sequence encoding a first cleavage domain, and a fifth nucleotide sequence encoding an output protein where the third segment contains one or more pseudouridines. In some embodiments, the methods combine the second and third segments. In some embodiments, the first segment contains a sequence for the 5′RNA cap which is followed by a sequence encoding the 5′ UTR which is followed by the first nucleotide sequence. In some embodiments where the second and third segments are combined, the second segment contains a sequence for the 5′RNA cap which is followed by a sequence encoding the 5′ UTR which is followed by the third nucleotide sequence. In some embodiments, the third segment contains the fourth and fifth nucleotide sequence followed by a sequence encoding the 3′ UTR followed by a sequence for a poly A tail.

The first segment, second segment, and third segments may be combined using any method deemed useful. In some embodiments, the first segment, second segment, and third segment are combined through ligation. The ligation may be performed by any ligase that ligates two or more RNA segments together. Ligase of interest include, without limitation, T4 DNA ligase, T4 RNA ligase 1, T4 RNA ligase 2, T3 DNA ligase, RtcB ligase, etc. In some embodiments, the ligase is a T4 DNA ligase. In some embodiments, the ligation is DNA oligo-mediated splint ligation. In some embodiments, the ligation is ssRNA ligation. In some embodiments, the DNA oligo-mediated splint ligation includes annealing a first DNA oligo to the first segment and the second segment, annealing a second DNA oligo to the second and third segment, and ligating the first segment, second segment, and the third segment using a ligase. The first DNA oligo brings the 3′ end of the first segment in ligatable proximity to the 5′ end of the second segment. The second DNA oligo brings the 3′ end of the second segment in ligatable proximity to the 5′ end of the third segment. The first DNA oligo anneals to the 3′ end of the first segment and the 5′ end of the second segment. The second DNA oligo anneals to the 3′ end of the second segment and the 5′ end of the third segment.

Methods for expressing a protein in a target cell may also be used to treat an individual for a disease or a condition. In the methods disclosed herein, the protein for expression in a target cell may promote the survival of the target cell or may promote the death of the cell. For instance, if the disease or condition is associated with a cell that is infected by a pathogen or a cancer cell then it may be desirable for the promotion of the death of such cells. In embodiments in which the death of the target cell is advantageous, output protein encoded by the sensor RNA may be any output protein that promotes the death of the cell. Output proteins that promote the death of the cell include, without limitation, a toxin, tumor necrosis factor alpha (TNFa), Fas ligand (FasL), a caspase such as caspase 1, caspase 2, caspase 3, caspase 4, caspase 5, caspase 6, caspase 7, caspase 8, caspase 9, caspase 10, caspase 11, caspase 12, caspase 13 or a variant thereof, etc.

In addition, if the disease or condition is associated with a cell that is infected by a pathogen or a cancer cell it may also be desirable to activate an immune cell to target the infected cell or cancer cell. Immune cells generally include white blood cells (leukocytes) which are derived from hematopoietic stem cells (HSC) produced in the bone marrow. Immune cells also include, e.g., lymphocytes (T cells, B cells, natural killer (NK) cells) and myeloid-derived cells (neutrophil, eosinophil, basophil, monocyte, macrophage, dendritic cells). T cells include all types of immune cells expressing CD3 including T-helper cells (CD4⁺ cells), cytotoxic T-cells (CD8⁺ cells), T-regulatory cells (Treg) and gamma-delta T cells. Cytotoxic cells include CD8⁺ T cells, natural-killer (NK) cells, and neutrophils, which cells are capable of mediating cytotoxicity responses.

In embodiments in which the activation of immune cell is advantageous, the target RNA that the sensor RNA is directed to may be a target RNA that is specifically expressed in an immune cell. In embodiments in which the activation of immune cell is advantageous, the sensor RNA may contain a sequence that encodes an output protein that activates or modulates the activity of the immune cell. Non-limiting examples of output proteins that activate immune cells include a chimeric antigen receptor, such as those described above, or a cytokine such as IL-1-like, IL-1α, IL-1β, IL-1RA, IL-18, CD132, IL-2, IL-4, IL-7, IL-9, IL-13, CD1243, 132, IL-15, CD131, IL-3, IL-5, GM-CSF, IL-6-like, IL-6, IL-11, G-CSF, IL-12, LIF, OSM, IL-10-like, IL-10, IL-20, IL-14, IL-16, IL-17, IFN-α, IFN-β, IFN-γ, CD154, LT-α, TNF-α, TNF-β, 4-1BBL, APRIL, CD70, CD153, CD178, GITRL, LIGHT, OX40L, TALL-1, TRAIL, TWEAK, TRANCE, TGF-01, TGF-02, TGF-β3, Epo, Tpo, Flt-3L, SCF, M-CSF, MSP, etc.

If the disease or condition is associated with the expression of a non-functional protein, a reduced functioning protein or a protein that has an aberrant activity in a disease state relative to a non-disease state then it may be desirable to have a sensor RNA that is targeted to the diseased cells where, upon contact with the diseased cell that contains the target RNA, the cell produces the output protein where the output protein is a fully functional form of the protein that is non-functioning, has reduced functionality or has aberrant functions.

If the disease or condition is associated with the degradation of a tissue, it may be desirable to promote the growth or regrowth of said tissue. In embodiments in which the disease or condition is associated with tissue degradation it may be desirable to have a sensor RNA that is targeted to the diseased cells where, upon contact with the diseased cell that contains the target RNA, the cell produces the output protein that promotes the growth or regrowth of the tissue. Non-limiting examples of output proteins that promote the growth or regrowth of the tissue include hormones and growth and differentiation factors including, without limitation, insulin, glucagon, growth hormone (GH), parathyroid hormone (PTH), growth hormone releasing factor (GHRF), follicle stimulating hormone (FSH), luteinizing hormone (LH), human chorionic gonadotropin (hCG), vascular endothelial growth factor (VEGF), angioproteinetins, angiostatin, granulocyte colony stimulating factor (GCSF), erythroproteinetin (EPO), connective tissue growth factor (CTGF), basic fibroblast growth factor (bFGF), acidic fibroblast growth factor (aFGF), epidermal growth factor (EGF), transforming growth factor .alpha. (TGFa), platelet-derived growth factor (PDGF), insulin growth factors I and II (IGF-1 and IGF-11), any one of the transforming growth factor 13-superfamily, including TGFI3, activins, inhibins, or any of the bone morphogenic proteins (BMP) including BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Given the diversity of cellular activities that may be modulated through the use of the subject sensor RNA, the instant methods of treatment may be utilized for a variety of applications. As non-limiting examples, the instant methods may find use in a treatment directed to a variety of diseases including but not limited to e.g., Acanthamoeba infection, Acinetobacter infection, Adenovirus infection, ADHD (Attention Deficit/Hyperactivity Disorder), AIDS (Acquired Immune Deficiency Syndrome), ALS (Amyotrophic Lateral Sclerosis), Alzheimer's Disease, Amebiasis, Intestinal (Entamoeba histolytica infection), Anaplasmosis, Human, Anemia, Angiostrongylus Infection, Animal-Related Diseases, Anisakis Infection (Anisakiasis), Anthrax, Aortic Aneurysm, Aortic Dissection, Arenavirus Infection, Arthritis (e.g., Childhood Arthritis, Fibromyalgia, Gout, Lupus (SLE) (Systemic lupus erythematosus), Osteoarthritis, Rheumatoid Arthritis, etc.), Ascaris Infection (Ascariasis), Aspergillus Infection (Aspergillosis), Asthma, Attention Deficit/Hyperactivity Disorder, Autism, Avian Influenza, B virus Infection (Herpes B virus), B. cepacia infection (Burkholderia cepacia Infection), Babesiosis (Babesia Infection), Bacterial Meningitis, Bacterial Vaginosis (BV), Balamuthia infection (Balamuthia mandrillaris infection), Balamuthia mandrillaris infection, Balantidiasis, Balantidium Infection (Balantidiasis), Baylisascaris Infection, Bilharzia, Birth Defects, Black Lung (Coal Workers' Pneumoconioses), Blastocystis hominis Infection, Blastocystis Infection, Blastomycosis, Bleeding Disorders, Blood Disorders, Body Lice (Pediculus humanus corporis), Borrelia burgdorferi Infection, Botulism (Clostridium botulinim), Bovine Spongiform Encephalopathy (BSE), Brainerd Diarrhea, Breast Cancer, Bronchiolitis, Bronchitis, Brucella Infection (Brucellosis), Brucellosis, Burkholderia cepacia Infection (B. cepacia infection), Burkholderia mallei, Burkholderia pseudomallei Infection, Campylobacter Infection (Campylobacteriosis), Campylobacteriosis, Cancer (e.g., Colorectal (Colon) Cancer, Gynecologic Cancers, Lung Cancer, Prostate Cancer, Skin Cancer, etc.), Candida Infection (Candidiasis), Candidiasis, Canine Flu, Capillaria Infection (Capillariasis), Capillariasis, Carbapenem resistant Klebsiella pneumonia (CRKP), Cat Flea Tapeworm, Cercarial Dermatitis, Cerebral Palsy, Cervical Cancer, Chagas Disease (Trypanosoma cruzi Infection), Chickenpox (Varicella Disease), Chikungunya Fever (CHIKV), Childhood Arthritis, German Measles (Rubella Virus), Measles, Mumps, Rotavirus Infection, Chlamydia (Chlamydia trachomatis Disease), Chlamydia pneumoniae Infection, Chlamydia trachomatis Disease, Cholera (Vibrio cholerae Infection), Chronic Fatigue Syndrome (CFS), Chronic Obstructive Pulmonary Disease (COPD), Ciguatera Fish Poisoning, Ciguatoxin, Classic Creutzfeldt-Jakob Disease, Clonorchiasis, Clonorchis Infection (Clonorchiasis), Clostridium botulinim, Clostridium difficile Infection, Clostridium perfringens infection, Clostridium tetani Infection, Clotting Disorders, CMV (Cytomegalovirus Infection), Coal Workers' Pneumoconioses, Coccidioidomycosis, Colorectal (Colon) Cancer, Common Cold, Conjunctivitis, Cooleys Anemia, COPD (Chronic Obstructive Pulmonary Disease), Corynebacterium diphtheriae Infection, Coxiella burnetii Infection, Creutzfeldt-Jakob Disease, CRKP (Carbapenem resistant Klebsiella pneumonia), Crohn's Disease, Cryptococcosis, Cryptosporidiosis, Cryptosporidium Infection (Cryptosporidiosis), Cyclospora Infection (Cyclosporiasis), Cyclosporiasis, Cysticercosis, Cystoisospora Infection (Cystoisosporaiasis), Cystoisosporaiasis, Cytomegalovirus Infection (CMV), Dengue Fever (DF), Dengue Hemorrhagic Fever (DHF), Dermatophytes, Dermopathy, Diabetes, Diamond Blackfan Anemia (DBA), Dientamoeba fragilis Infection, Diphtheria (Corynebacterium diphtheriae Infection), Diphyllobothriasis, Diphyllobothrium Infection (Diphyllobothriasis), Dipylidium Infection, Dog Flea Tapeworm, Down Syndrome (Trisomy 21), Dracunculiasis, Dwarf Tapeworm (Hymenolepis Infection), E. coli Infection (Escherichia coli Infection), Ear Infection (Otitis Media), Eastern Equine Encephalitis (EEE), Ebola Hemorrhagic Fever, Echinococcosis, Ehrlichiosis, Elephantiasis, Encephalitis (Mosquito-Borne and Tick-Borne), Entamoeba histolytica infection, Enterobius vermicularis Infection, Enterovirus Infections (Non-Polio), Epidemic Typhus, Epilepsy, Epstein-Barr Virus Infection (EBV Infection), Escherichia coli Infection, Extensively Drug-Resistant TB (XDR TB), Fasciola Infection (Fascioliasis), Fasciolopsis Infection (Fasciolopsiasis), Fibromyalgia, Fifth Disease (Parvovirus B19 Infection), Flavorings-Related Lung Disease, Folliculitis, Food-Related Diseases, Clostridium perfringens infection, Fragile X Syndrome, Francisella tularensis Infection, Genital Candidiasis (Vulvovaginal Candidiasis (VVC)), Genital Herpes (Herpes Simplex Virus Infection), Genital Warts, German Measles (Rubella Virus), Giardia Infection (Giardiasis), Glanders (Burkholderia mallei), Gnathostoma Infection, Gnathostomiasis (Gnathostoma Infection), Gonorrhea (Neisseria gonorrhoeae Infection), Gout, Granulomatous amebic encephalitis (GAE), Group A Strep Infection (GAS) (Group A Streptococcal Infection), Group B Strep Infection (GBS) (Group B Streptococcal Infection), Guinea Worm Disease (Dracunculiasis), Gynecologic Cancers (e.g., Cervical Cancer, Ovarian Cancer, Uterine Cancer, Vaginal and Vulvar Cancers, etc.), H1N1 Flu, Haemophilus influenzae Infection (Hib Infection), Hand, Foot, and Mouth Disease (HFMD), Hansen's Disease, Hantavirus Pulmonary Syndrome (HPS), Head Lice (Pediculus humanus capitis), Heart Disease (Cardiovascular Health), Heat Stress, Hemochromatosis, Hemophilia, Hendra Virus Infection, Herpes B virus, Herpes Simplex Virus Infection, Heterophyes Infection (Heterophyiasis), Hib Infection (Haemophilus influenzae Infection), High Blood Pressure, Histoplasma capsulatum Disease, Histoplasmosis (Histoplasma capsulatum Disease), Hot Tub Rash (Pseudomonas dermatitis Infection), HPV Infection (Human Papillomavirus Infection), Human Ehrlichiosis, Human Immunodeficiency Virus, Human Papillomavirus Infection (HPV Infection), Hymenolepis Infection, Hypertension, Hyperthermia, Hypothermia, Impetigo, Infectious Mononucleosis, Inflammatory Bowel Disease (IBD), Influenza, Avian Influenza, H1N1 Flu, Pandemic Flu, Seasonal Flu, Swine Influenza, Invasive Candidiasis, Iron Overload (Hemochromatosis), Isospora Infection (Isosporiasis), Japanese Encephalitis, Jaundice, K. pneumoniae (Klebsiella pneumoniae), Kala-Azar, Kawasaki Syndrome (KS), Kernicterus, Klebsiella pneumoniae (K. pneumoniae), La Crosse Encephalitis (LAC), La Crosse Encephalitis virus (LACV), Lassa Fever, Latex Allergies, Lead Poisoning, Legionnaires' Disease (Legionellosis), Leishmania Infection (Leishmaniasis), Leprosy, Leptospira Infection (Leptospirosis), Leptospirosis, Leukemia, Lice, Listeria Infection (Listeriosis), Listeriosis, Liver Disease and Hepatitis, Loa Infection, Lockjaw, Lou Gehrig's Disease, Lung Cancer, Lupus (SLE) (Systemic lupus erythematosus), Lyme Disease (Borrelia burgdorferi Infection), Lymphatic Filariasis, Lymphedema, Lymphocytic Choriomeningitis (LCMV), Lymphogranuloma venereum Infection (LGV), Malaria, Marburg Hemorrhagic Fever, Measles, Melioidosis (Burkholderia pseudomallei Infection), Meningitis (Meningococcal Disease), Meningococcal Disease, Methicillin Resistant Staphylococcus aureus (MRSA), Micronutrient Malnutrition, Microsporidia Infection, Molluscum Contagiosum, Monkey B virus, Monkeypox, Morgellons, Mosquito-Borne Diseases, Mucormycosis, Multidrug-Resistant TB (MDR TB), Mumps, Mycobacterium abscessus Infection, Mycobacterium avium Complex (MAC), Mycoplasma pneumoniae Infection, Myiasis, Naegleria Infection (Primary Amebic Meningoencephalitis (PAM)), Necrotizing Fasciitis, Neglected Tropical Diseases (NTD), Neisseria gonorrhoeae Infection, Neurocysticercosis, New Variant Creutzfeldt-Jakob Disease, Newborn Jaundice (Kernicterus), Nipah Virus Encephalitis, Nocardiosis, Non-Polio Enterovirus Infections, Nonpathogenic (Harmless) Intestinal Protozoa, Norovirus Infection, Norwalk-like Viruses (NLV), Novel H1N1 Flu, Onchocerciasis, Opisthorchis Infection, Oral Cancer, Orf Virus, Oropharyngeal Candidiasis (OPC), Osteoarthritis (OA), Osteoporosis, Otitis Media, Ovarian Cancer, Pandemic Flu, Paragonimiasis, Paragonimus Infection (Paragonimiasis), Parasitic Diseases, Parvovirus B19 Infection, Pediculus humanus capitis, Pediculus humanus corporis, Pelvic Inflammatory Disease (PID), Peripheral Arterial Disease (PAD), Pertussis, Phthiriasis, Pink Eye (Conjunctivitis), Pinworm Infection (Enterobius vermicularis Infection), Plague (Yersinia pestis Infection), Pneumocystis jirovecii Pneumonia, Pneumonia, Polio Infection (Poliomyelitis Infection), Pontiac Fever, Prion Diseases (Transmissible spongiform encephalopathies (TSEs)), Prostate Cancer, Pseudomonas dermatitis Infection, Psittacosis, Pubic Lice (Phthiriasis), Pulmonary Hypertension, Q Fever (Coxiella burnetii Infection), Rabies, Raccoon Roundworm Infection (Baylisascaris Infection), Rat-Bite Fever (RBF) (Streptobacillus moniliformis Infection), Recreational Water Illness (RWI), Relapsing Fever, Respiratory Syncytial Virus Infection (RSV), Rheumatoid Arthritis (RA), Rickettsia rickettsii Infection, Rift Valley Fever (RVF), Ringworm (Dermatophytes), Ringworm in Animals, River Blindness (Onchocerciasis), Rocky Mountain Spotted Fever (RMSF) (Rickettsia rickettsii Infection), Rotavirus Infection, RVF (Rift Valley Fever), RWI (Recreational Water Illness), Salmonella Infection (Salmonellosis), Scabies, Scarlet Fever, Schistosomiasis (Schistosoma Infection), Seasonal Flu, Severe Acute Respiratory Syndrome, Sexually Transmitted Diseases (STDs) (e.g., Bacterial Vaginosis (BV), Chlamydia, Genital Herpes, Gonorrhea, Human Papillomavirus Infection, Pelvic Inflammatory Disease, Syphilis, Trichomoniasis, HIV/AIDS, etc.), Shigella Infection (Shigellosis), Shingles (Varicella Zoster Virus (VZV)), Sickle Cell Disease, Single Gene Disorders, Sinus Infection (Sinusitus), Skin Cancer, Sleeping Sickness (African Trypanosomiasis), Smallpox (Variola Major and Variola Minor), Sore Mouth Infection (Orf Virus), Southern Tick-Associated Rash Illness (STARI), Spina Bifida (Myelomeningocele), Sporotrichosis, Spotted Fever Group Rickettsia (SFGR), St. Louis Encephalitis, Staphylococcus aureus Infection, Streptobacillus moniliformis Infection, Streptococcal Diseases, Streptococcus pneumoniae Infection, Stroke, Strongyloides Infection (Strongyloidiasis), Sudden Infant Death Syndrome (SIDS), Swimmer's Itch (Cercarial Dermatitis), Swine Influenza, Syphilis (Treponema pallidum Infection), Systemic lupus erythematosus, Tapeworm Infection (Taenia Infection), Testicular Cancer, Tetanus Disease (Clostridium tetani Infection), Thrush (Oropharyngeal Candidiasis (OPC)), Tick-borne Relapsing Fever, Tickborne Diseases (e.g., Anaplasmosis, Babesiosis, Ehrlichiosis, Lyme Disease, Tourette Syndrome (TS), Toxic Shock Syndrome (TSS), Toxocariasis (Toxocara Infection), Toxoplasmosis (Toxoplasma Infection), Trachoma Infection, Transmissible spongiform encephalopathies (TSEs), Traumatic Brain Injury (TBI), Trichinellosis (Trichinosis), Trichomoniasis (Trichomonas Infection), Tuberculosis (TB) (Mycobacterium tuberculosis Infection), Tularemia (Francisella tularensis Infection), Typhoid Fever (Salmonella typhi Infection), Uterine Cancer, Vaginal and Vulvar Cancers, Vancomycin-Intermediate/Resistant Staphylococcus aureus Infections (VISA/VRSA), Vancomycin-resistant Enterococci Infection (VRE), Variant Creutzfeldt-Jakob Disease (vCJD), Varicella-Zoster Virus Infection, Variola Major and Variola Minor, Vibrio cholerae Infection, Vibrio parahaemolyticus Infection, Vibrio vulnificus Infection, Viral Gastroenteritis, Viral Hemorrhagic Fevers (VHF), Viral Hepatitis, Viral Meningitis (Aseptic Meningitis), Von Willebrand Disease, Vulvovaginal Candidiasis (VVC), West Nile Virus Infection, Western Equine Encephalitis Infection, Whipworm Infection (Trichuriasis), Whitmore's Disease, Whooping Cough, Xenotropic Murine Leukemia Virus-related Virus Infection, Yellow Fever, Yersinia pestis Infection, Yersiniosis (Yersinia enterocolitica Infection), Zoonotic Hookworm, Zygomycosis, and the like.

In some instances, methods of treatment utilizing one or more sensor RNAs of the instant disclosure may find use in treating a cancer. Cancers, the treatment of which may include the use of sensor RNAs of the instant disclosure, will vary and may include but are not limited to e.g., Acute Lymphoblastic Leukemia (ALL), Acute Myeloid Leukemia (AML), Adrenocortical Carcinoma, AIDS-Related Cancers (e.g., Kaposi Sarcoma, Lymphoma, etc.), Anal Cancer, Appendix Cancer, Astrocytomas, Atypical Teratoid/Rhabdoid Tumor, Basal Cell Carcinoma, Bile Duct Cancer (Extrahepatic), Bladder Cancer, Bone Cancer (e.g., Ewing Sarcoma, Osteosarcoma and Malignant Fibrous Histiocytoma, etc.), Brain Stem Glioma, Brain Tumors (e.g., Astrocytomas, Central Nervous System Embryonal Tumors, Central Nervous System Germ Cell Tumors, Craniopharyngioma, Ependymoma, etc.), Breast Cancer (e.g., female breast cancer, male breast cancer, childhood breast cancer, etc.), Bronchial Tumors, Burkitt Lymphoma, Carcinoid Tumor (e.g., Childhood, Gastrointestinal, etc.), Carcinoma of Unknown Primary, Cardiac (Heart) Tumors, Central Nervous System (e.g., Atypical Teratoid/Rhabdoid Tumor, Embryonal Tumors, Germ Cell Tumor, Lymphoma, etc.), Cervical Cancer, Childhood Cancers, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct (e.g., Bile Duct, Extrahepatic, etc.), Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer (e.g., Intraocular Melanoma, Retinoblastoma, etc.), Fibrous Histiocytoma of Bone (e.g., Malignant, Osteosarcoma, ect.), Gallbladder Cancer, Gastric (Stomach) Cancer, Gastrointestinal Carcinoid Tumor, Gastrointestinal Stromal Tumors (GIST), Germ Cell Tumor (e.g., Extracranial, Extragonadal, Ovarian, Testicular, etc.), Gestational Trophoblastic Disease, Glioma, Hairy Cell Leukemia, Head and Neck Cancer, Heart Cancer, Hepatocellular (Liver) Cancer, Histiocytosis (e.g., Langerhans Cell, etc.), Hodgkin Lymphoma, Hypopharyngeal Cancer, Intraocular Melanoma, Islet Cell Tumors (e.g., Pancreatic Neuroendocrine Tumors, etc.), Kaposi Sarcoma, Kidney Cancer (e.g., Renal Cell, Wilms Tumor, Childhood Kidney Tumors, etc.), Langerhans Cell Histiocytosis, Laryngeal Cancer, Leukemia (e.g., Acute Lymphoblastic (ALL), Acute Myeloid (AML), Chronic Lymphocytic (CLL), Chronic Myelogenous (CML), Hairy Cell, etc.), Lip and Oral Cavity Cancer, Liver Cancer (Primary), Lobular Carcinoma In Situ (LCIS), Lung Cancer (e.g., Non-Small Cell, Small Cell, etc.), Lymphoma (e.g., AIDS-Related, Burkitt, Cutaneous T-Cell, Hodgkin, Non-Hodgkin, Primary Central Nervous System (CNS), etc.), Macroglobulinemia (e.g., Waldenstrom, etc.), Male Breast Cancer, Malignant Fibrous Histiocytoma of Bone and Osteosarcoma, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia (e.g., Chronic (CML), etc.), Myeloid Leukemia (e.g., Acute (AML), etc.), Myeloproliferative Neoplasms (e.g., Chronic, etc.), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer (e.g., Lip, etc.), Oropharyngeal Cancer, Osteosarcoma and Malignant Fibrous Histiocytoma of Bone, Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (e.g., Ewing, Kaposi, Osteosarcoma, Rhabdomyosarcoma, Soft Tissue, Uterine, etc.), Sézary Syndrome, Skin Cancer (e.g., Childhood, Melanoma, Merkel Cell Carcinoma, Nonmelanoma, etc.), Small Cell Lung Cancer, Small Intestine Cancer, Soft Tissue Sarcoma, Squamous Cell Carcinoma, Squamous Neck Cancer (e.g., with Occult Primary, Metastatic, etc.), Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer (e.g., Endometrial, etc.), Uterine Sarcoma, Vaginal Cancer, Vulvar Cancer, Waldenstrom Macroglobulinemia, Wilms Tumor, and the like.

Also included are compositions for practicing the methods are described in the present disclosure. In general, subject compositions may have sensor RNA as described above in addition to a pharmaceutically acceptable excipient. In some embodiments, the subject compositions contain a secondary agent for treating any of the diseases or conditions described above.

Compositions of the present disclosure can be administered by any suitable means, including topical, oral, parenteral, intrapulmonary, and intranasal. Parenteral infusions include intramuscular, intravenous (bolus or slow drip), intraarterial, intraperitoneal, intrathecal or subcutaneous administration. An agent can be administered in any manner which is medically acceptable. This may include injections, by parenteral routes such as intravenous, intravascular, intraarterial, subcutaneous, intramuscular, intratumor, intraperitoneal, intraventricular, intraepidural, or others as well as oral, nasal, ophthalmic, rectal, or topical. Sustained release administration is also specifically included in the disclosure, by such means as depot injections or erodible implants.

As noted above, sensor RNA can be formulated with an a pharmaceutically acceptable carrier (one or more organic or inorganic ingredients, natural or synthetic, with which a subject agent can be combined to facilitate its application). A suitable carrier includes sterile saline although other aqueous and non-aqueous isotonic sterile solutions and sterile suspensions known to be pharmaceutically acceptable are known to those of ordinary skill in the art. An “effective amount” refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one of ordinary skill in the art employing such factors and using no more than routine experimentation.

The composition may be administered in a unit dosage form and may be prepared by any methods well known in the art. Such methods include combining agent with a pharmaceutically acceptable carrier or diluent which constitutes one or more accessory ingredients. A pharmaceutically acceptable carrier can be selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type can be generally chosen based on the type of administration being used.

Depending on the individual and condition being treated and on the administration route, the active agent may be administered in dosages of 0.01 mg to 500 mg/kg body weight per day, e.g. about 20 mg/day for an average person. Dosages will be appropriately adjusted for pediatric formulation.

In some embodiments, the composition can be formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the composition may further include a preservative. Suitable preservatives include, but are not limited to, a benzyl alcohol, phenol, chlorobutanol, benzalkonium chloride, and the like. In many cases, the composition can be stored at about 4° C. Pharmaceutical compositions may also be lyophilized, in which case they generally include cryoprotectants such as sucrose, trehalose, lactose, maltose, mannitol, and the like. Lyophilized formulations can be stored over extended periods of time, even at ambient temperatures.

Compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The compositions of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

As described above, the composition may also contain a secondary agent for treatment of any of the diseases or condition described above. When the disease or condition is cancer, the secondary agent may be a chemotherapeutic agent. Chemotherapeutic agents that find use in the present disclosure include, without limitation, Abitrexate (Methotrexate Injection), Abraxane (Paclitaxel Injection), Adcetris (Brentuximab Vedotin Injection), Adriamycin (Doxorubicin), Adrucil Injection (5-FU (fluorouracil)), Afinitor (Everolimus), Afinitor Disperz (Everolimus), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Bexxar (Tositumomab), BiCNU (Carmustine), Blenoxane (Bleomycin), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Dacogen (Decitabine), DaunoXome (Daunorubicin Lipid Complex Injection), Decadron (Dexamethasone), DepoCyt (Cytarabine Lipid Complex Injection), Dexamethasone Intensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Eligard (Leuprolide), Ellence (Ellence (epirubicin)), Eloxatin (Eloxatin (oxaliplatin)), Elspar (Asparaginase), Emcyt (Estramustine), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), lclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leukeran (Chlorambucil), Leukine (Sargramostim), Leustatin (Cladribine), Lupron (Leuprolide), Lupron Depot (Leuprolide), Lupron DepotPED (Leuprolide), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinolAQ (Cisplatin), PlatinolAQ (Cisplatin Injection), Pomalyst (Pomalidomide), Prednisone Intensol (Prednisone), Proleukin (Aldesleukin), Purinethol (Mercaptopurine), Reclast (Zoledronic acid), Revlimid (Lenalidomide), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Trelstar (Triptorelin Injection), Trexall (Methotrexate), Trisenox (Arsenic trioxide), Tykerb (lapatinib), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (Ibritumomab Tiuxetan), Zoladex (Goserelin), Zolinza (Vorinostat), Zometa (Zoledronic acid), Zortress (Everolimus), Zytiga (Abiraterone), Nimotuzumab and immune checkpoint inhibitors such as nivolumab, pembrolizumab/MK-3475, pidilizumab and AMP-224 targeting PD-1; and BMS-935559, MEDI4736, MPDL3280A and MSB0010718C targeting PD-L1 and those targeting CTLA-4 such as ipilimumab.

When the disease or condition is associated with an infection, the secondary agent may be an antibiotic. Antibiotics that find use in the present disclosure include, without limitation, antibiotics with the classes of aminoglycosides; carbapenems; and the like; penicillins, e.g. penicillin G, penicillin V, methicillin, oxacillin, carbenicillin, nafcillin, ampicillin, etc. penicillins in combination with β-lactamase inhibitors, cephalosporins, e.g. cefaclor, cefazolin, cefuroxime, moxalactam, etc:; tetracyclines; cephalosporins; quinolones; lincomycins; macrolides; sulfonamides; glycopeptides including the anti-infective antibiotics vancomycin, teicoplanin, telavancin, ramoplanin and decaplanin. Derivatives of vancomycin include, for example, oritavancin and dalbavancin (both lipoglycopeptides). Telavancin is a semi-synthetic lipoglycopeptide derivative of vancomycin (approved by FDA in 2009). Other vancomycin analogs are disclosed, for example, in WO 2015022335 A1 and Chen et al. (2003) PNAS 100(10): 5658-5663, each herein specifically incorporated by reference. Non-limiting examples of antibiotics include vancomycin, linezolid, azithromycin, daptomycin, colistin, eperezolid, fusidic acid, rifampicin, tetracyclin, fidaxomicin, clindamycin, lincomycin, rifalazil, and clarithromycin.

In some embodiments, an RNA sensor as described herein can comprise any of the sequences in Table A below, or portions thereof.

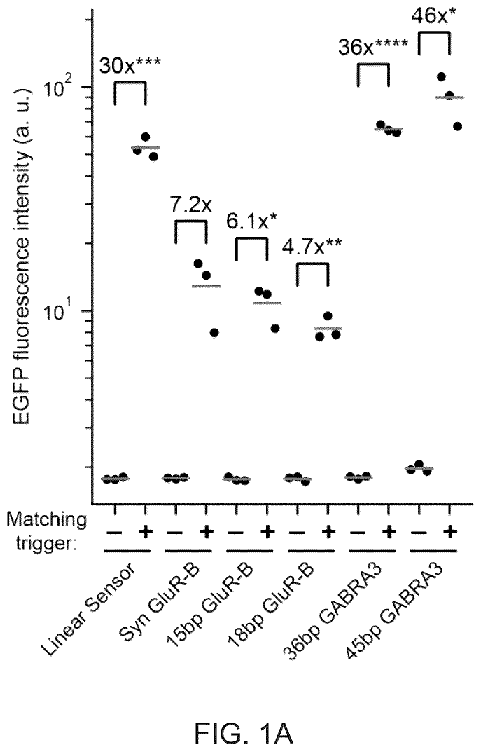

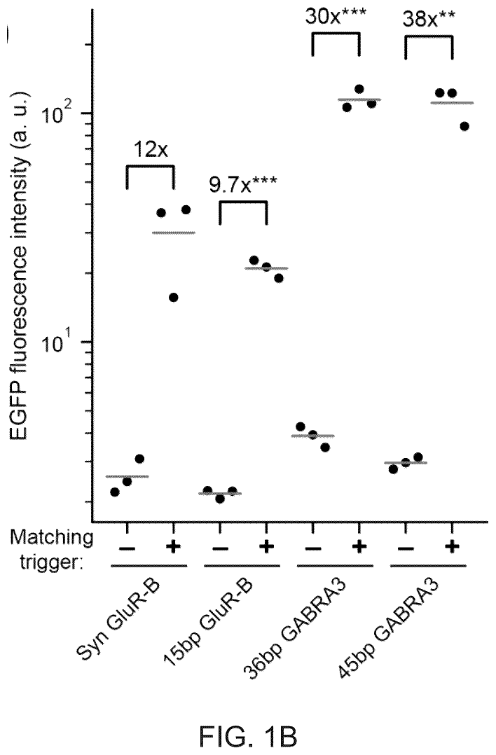

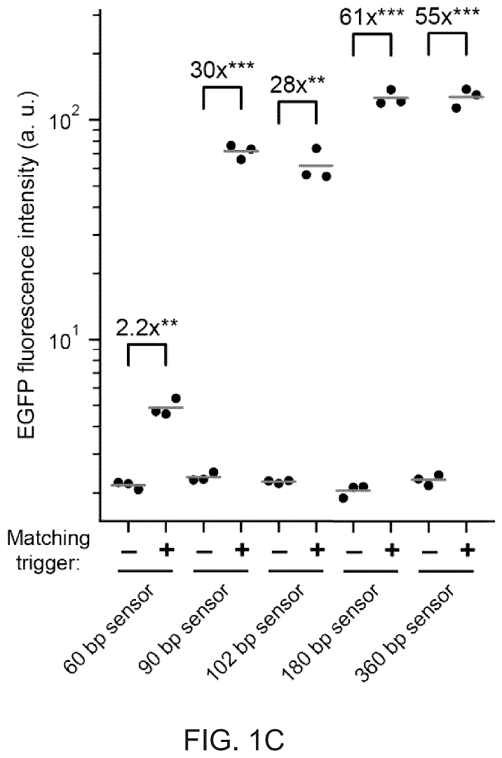

TABLE A
Sequences usable in RNA sensors described herein.

| | | SEQ ID |
|---|---|---|
| Description | Sequence | NO: |
| FIG. 1A Trigger | CCCUGCCUUGUCUACCAAACCCUCAGGCAGGAUUUGCGCGCCGA | 50 |
| sequence: | AGUGCUCCGCGGAGAGCAGGUGUAGCGGGAAAUAAGUAACUGA | |
| | UCUCGUCGACAUGAUCGUUAAGGGUGGGCGCGGUUGCGUAAUG | |
| | CUAAGAUAGCUCUCUAUUUGCACCAGUGGUCUAUCGGUACGUA | |
| | AGGCCCAACAAUGACGCGGAUAGAAGGAAACACUCAUCCGUGU | |
| | CGACUUGGUCCACACGCGUACUCCGCGGAACAUACUUCGGUGUC | |
| | GAGUGGUAAUAGAUGCCGGUUGUGGCAGAGUCUGAGGAAUACU | |
| | CACGUAGCAACAGCUUUGAAAGUAGAAGAAACCUGCCUGACUG | |
| | UCGGCGAGUGAACC | |
| FIG. 1A Linear | ACCAAGUCGACACGGAUGAGUGUUUCCUUCUAUCCGCGUCAUU | 51 |
| sensor: | GUUaGGCCUUACGUACCGAUAGACCACUGGUGCAAAUAGAGAG | |
| | CUAU | |
| FIG. 1A Syn GlurR- | ACCAAGUCGACACGGAUGAGUGUUUCCUUCUAUCCGCGUCAUU | 52 |
| B sensor | GUUCCGUUUAGGUGGGUGGAAUAGUAAUACAAAGUAUCCCACC | |
| | UACCCAGACGUgGGCCUUACGUACCGAUAGACCACUGGUGCAAA | |
| | UAGAGAGCUAU | |
| FIG. 1A 15 bp GluR- | ACCAAGUCGACACGGAUGAGUGUUUCCUUCUAUCCGCGUCAUU | 53 |
| B sensor | GUacCAUUUAGGUGGGUGGGCUAACCACCUACCCaGAUGccUgGG | |
| | CCUUACGUACCGAUAGACCACUGGUGCAAAUAGAGAGCUAU | |
| FIG. 1A 18 bp GluR- | ACCAAGUCGACACGGAUGAGUGUUUCCUUCUAUCCGCGUCAUU | 54 |
| B sensor | GUacCAUUUAGGUGGGUGGAAUGCUAAAUCCCACCUACCCaGAU | |
| | GccUgGGCCUUACGUACCGAUAGACCACUGGUGCAAAUAGAGAG | |
| | CUAU | |
| FIG. 1A 36 bp | ACCAAGUCGACACGGAUGAGUGUUUCCUUCUAUCCGCGUCAUU | 55 |
| GABRA3 sensor | GUAUGCGACGGCCAUGGACUGGUUCAUAGCCGUCUGUUUgGGC | |
| | CUUACGUACCGAUAGACCACUGGUGCAAAUAGAGAGCUAU | |
| FIG. 1A 45 bp | ACCAAGUCGACACGGAUGAGUGUUUCCUUCUAUCCGCGUCAUU | 56 |
| GABRA3 sensor | GUUGGCAUAUGCGACGGCCAUGGACUGGUUCAUAGCCGUCUGU | |
| | UAUGUgGGCCUUACGUACCGAUAGACCACUGGUGCAAAUAGAG | |
| | AGCUAU | |
| FIG. 1B Trigger | UGGAUUUAUGUUGUAUAGAUUAUAUUGAGACAAAAUUAUCUA | 57 |
| sequence: | UUUGUAUAUAUACAUAACAGGGUAAAUUAUUCAGUUAAGAAA | |
| | AAAUAAUUUUAUGAACUGCAUGUAUAAAUGAAGUUUAUACAG | |
| | UACAGUGGUUCUACAAUCUAUUUAUUGGACAUAUCCAUGACCU | |
| | GAAAGGAAACAGUCAUUUGCGCACAACUUUAAAAGUCUGCAUU | |
| | ACAUUCCUCGAUAAUGUUGUGGUUUGUUGCCGUUGCCAAGAAU | |
| | UGAAAACAAAAAGUUUAAAAAAAAUAAUAAUAAAUUGCAUGC | |
| | UGCUUUAAUUGUGAAUUGAUAAUAAACUGUCCCUCUUUCAGAA | |
| | AACAGAUUAAAAAAACAAAAAACAAAAAAAAAAAAACAAAAAA | |
| | CAAAAACAAAAAUUGGAACCAAAACAUUCCGUUUACAUUUUAG | |
| | ACACUAAGUAUCUUCGUUCUUGUUAGUACUCUGUUUUACUGCU | |
| | UUCGACUUCUGAUAGCGUUGGAAUUAAAACAAUGUCAAGGUGC | |
| | UGUUGUCAUUGCUUUACUGGCGUAAGGGACGGGGAAUGGGAGG | |
| | GGUAGAUUUCUGUUUGUUUUGUGUUUUAUUUUGUUUGUUUGU | |
| | UUGUUUUGUUUUUUAGUUCCACCCGGAGUAGGGAUGGAGAAAA | |
| | UUUCUUCACUAUCCAUUCUGGUUGAUAAAGCGUUACAUUUGUA | |
| | UGUUGUAAAGAUGUUUGCAAAAUCCAAUCAGAUGACUGGAAAA | |
| | CAAAUAAAAAUUAAGGCAACUGAAUAAAAUGCUCACACUCCAC | |
| | UGCCCAUGAUGUAUCUCCCUGGUCCCCCUCAGCUCACUCUUCUG | |
| | GCAUGGGUCAGGGAAAAUUGCUUUUAUUGGAAAGACCAGCAUU | |
| | UGUUCAAAGCAUACUCUUUCCCUCCCUCCUCCCAUUUUGGUCCC | |
| | UUCUUUUUGUUUUGUUUUAAGAAAGAAAAUUAAGUUGCGCGCU | |
| | UUAAAAUAUUUUACUACUGCUACAAACAGAUGAACAAUGUGUG | |
| | UCAUUUUAUGACACUCAUGGAAAACAGUGAUUUUUUUUUACCC | |
| | UAAAGAAAAACAAAUAAAAAUAACCCAAAAUAUUCUUUUUUUA | |
| | AAAGGCAUAAAUAUUGGGUAAAUUGUAAUAUGGCCUAACAGUG | |
| | UUUGCAGAUAAAAGUUAUUGUAUACACCCAGAUACUUAGAUAA | |
| | GAGCAGGGAUCCACACUGCCAUUGAAAUAGGACUGAAUGGCCC | |
| | UGCGGAGGCUAAGUGGAGCUGACAUACUAUUUCCUGGCAGUGC | |
| | AGGAGGAAUUUCUGAGUGGCCAUCCUAAGGUCUAGGAUGGAGG | |
| | UGGGGAAUGGUACUUGAGACAUUCCUAAAGGAAGGCUCGGAAG | |
| | CACCCUUCAGAGCAGGCUCUGGAAUGAUGUGUCAAGUUGCUUA | |
| | GGCCUUCUGCUUUAAGUGCCUACAUUACCUAACAGUGCUCAAG | |
| | AGGUUCUCGAUUGGAGAACCACACUCAAAUCCAUUUAUAGCCU | |
| | CCAUCCCAUUUCUAAAUAAUUGUGUAUAAAGUUGGAUUAACCU | |
| | GGAGCAACUUUGGAUCCAAAUAUGGCACAGCAAUAAUGAUAUU | |
| | AAUGCAGCAUGAUGGGAAAUGUUUGCUGUGAAGAGAAUUGAU | |
| | UUGCUUUGAGCUUAGACUUCAGGAAGCCUAGGUUUUUUAUUUU | |
| | UUUAUUUUUGAGACAUUUUGGUAAAAGGAAAAAAAGAAAACA | |
| | AACAAACAAACAAACAAAACCAGAAAAAGCAUCAAAACUCAGG | |
| | CAGAAUGAGCAAUGUCUGAAAGGGCUAGAAAAACAAGACAUAG | |
| | CAAGGUGCUUUCACUGUGAAAGAGACAAGAACACAGGAGGAAA | |
| | UAUUGCUUCAGUGAAGAGCACAGACGGCUCCUGCCAAUUUAUU | |
| | ACAAGAGUCCCGUCUGUACUUUACCCUUUGGGGUUAGAAGUCA | |
| | AGUUGGAAGCCUGAAUGAAUGGACCCAAUGAGAACUAGUGUUA | |
| | AGCCCAUUUCCCUAGUCAGGUUUUUUUCAAGCGUGAAUGUGUU | |
| | AGUGGUUACUCUCCUGGGUUCCUGAGCAUCAGAAAAAAAAAAA | |
| | AAAAGAGGCAAACAAUCGCUUCAUCUUAGGAGUGGAAAGGAAA | |
| | CAGAAGUGGACGUCCGCUGUGACUCAGGGAGUGAAGAUACCAU | |
| | CAGCAAAUAGUUUCUUUUUUGUUCAUUCGUUCCUUUCGAGUUA | |
| | GCCUGUCUUUUGGAAUACCACUGAAUAUGCUGUUUUUGAAAGA | |
| | CUUCAUGUAGCAUAGAUUGUUUUGUGCCGUUUACCAAAUUAAC | |
| | CUUUGUCAUCGUUUUUUAACCUAUUCAGGAAUGCUUGGAAUAU | |
| | CUGCUCUAUGUUAACUUUUUGCAGCUUCAUUCUGAGAGACAUU | |
| | AGUCAAACAAACAAAAGGAUCCCCAUCACAAUCUUACAGUACU | |
| | GCAAGGGCCAGGUCUGUUAAUCGGCUUCACAGGAGACAUCAGC | |
| | AAUUGUGUGGUCAGUGGCUGGCUCUCUUACCCACUAAGAUACA | |
| | UCAUAGCUACAUGUUGGUGGUUUAUGUUGACCUGAGAUUUAUU | |
| | UGUUAAAAUCUCUUCUUCGUUUCUGUUCGUUCUGGUUCUGUUC | |
| | UGUUCUGUUCUGUUCUGUUUUGGUUUUAAAGUCUUGCUGUGGU | |
| | CUCUUGUUGGCAGAAAUGUUUUAUGCAUGGCAGCAGGCCUGUU | |
| | GCUUUUUUAUAGUGAUUCCCAUUGAAACUGUAAGUAAAUGUCU | |
| | GUGGCCUUGUUCUCUCUAUGGUAAAGAUAUUAUUCACCAUGUA | |
| | AAACAAGAAAAAAUAUUUAUUGUAUUUUAGUAUAUUUAUAUA | |
| | AUUAUGUUAUUGAAAAAAUUGGCAUUAAAACUUAACCACAUCA | |
| | GAAGCCUAUUGUAAAUACAGGUUCUAUUUAAGUGUACCAAUUA | |
| | ACAUAUAAUAUAUGUUUUAAAUA | |
| FIG. 1B Syn GlurR- | CAAACAUCUUUACAACAUACAAAUGUAACGCUUUAUCAACCAG | 58 |
| B sensor | AAUCCGUUUAGGUGGGUGGAAUAGUAAUACAAAGUAUCCCACC | |
| | UACCCAGACGUGGAUAGUGAAGAAAUUUUCUCCAUCCCUACUC | |
| | CGGGUGGAACUA | |
| FIG. 1B 15 bp GluR- | CAAACAUCUUUACAACAUACAAAUGUAACGCUUUAUCAACCAG | 59 |
| B sensor | AAacCAUUUAGGUGGGUGGGCUAACCACCUACCCaGAUGccUGGA | |
| | UAGUGAAGAAAUUUUCUCCAUCCCUACUCCGGGUGGAACUA | |
| FIG. 1B 36 bp | CAAACAUCUUUACAACAUACAAAUGUAACGCUUUAUCAACCAG | 60 |
| GABRA3 sensor | AAAUGCGACGGCCAUGGACUGGUUCAUAGCCGUCUGUUUGGAU | |
| | AGUGAAGAAAUUUUCUCCAUCCCUACUCCGGGUGGAACUA | |
| FIG. 1B 45 bp | CAAACAUCUUUACAACAUACAAAUGUAACGCUUUAUCAACCAG | 61 |
| GABRA3 sensor | AAUGGCAUAUGCGACGGCCAUGGACUGGUUCAUAGCCGUCUGU | |
| | UAUGUGGAUAGUGAAGAAAUUUUCUCCAUCCCUACUCCGGGUG | |
| | GAACUA | |

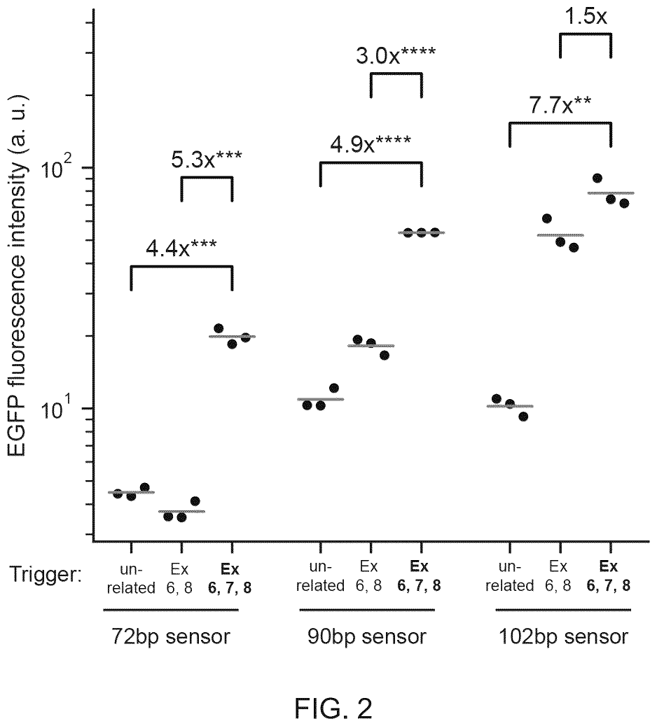

FIG. 1c

| | | |
|---|---|---|
| FIG. 1C Trigger | CCCUGCCUUGUCUACCAAACCCUCAGGCAGGAUUUGCGCGCCGA | 62 |
| sequence: | AGUGCUCCGCGGAGAGCAGGUGUAGCGGGAAAUAAGUAACUGA | |
| | UCUCGUCGACAUGAUCGUUAAGGGUGGGCGCGGUUGCGUAAUG | |
| | CUAAGAUAGCUCUCUAUUUGCACCAGUGGUCUAUCGGUACGUA | |
| | AGGCCCAACAAUGACGCGGAUAGAAGGAAACACUCAUCCGUGU | |
| | CGACUUGGUCCACACGCGUACUCCGCGGAACAUACUUCGGUGUC | |
| | GAGUGGUAAUAGAUGCCGGUUGUGGCAGAGUCUGAGGAAUACU | |
| | CACGUAGCAACAGCUUUGAAAGUAGAAGAAACCUGCCUGACUG | |
| | UCGGCGAGUGAACC | |
| FIG. 1C 60 bp sensor | AUGAGUGUUUCCUUCUAUCCGCGUCAUUGUUGGCAUAUGCGAC | 63 |
| | GGCCAUGGACUGGUUCAUAGCCGUCUGUUAUGUgGGCCUUACG | |
| | UACCGAUAGACCACUGGUG | |
| FIG. 1C 90 bp sensor | ACCAAGUCGACACGGAUGAGUGUUUCCUUCUAUCCGCGUCAUU | 64 |
| | GUUGGCAUAUGCGACGGCCAUGGACUGGUUCAUAGCCGUCUGU | |
| | UAUGUgGGCCUUACGUACCGAUAGACCACUGGUGCAAAUAGAG | |
| | AGCUAU | |
| FIG. 1C 102 bp | GUGUgGACCAAGUCGACACGGAUGAGUGUUUCCUUCUAUCCGC | 65 |
| sensor | GUCAUUGUUGGCAUAUGCGACGGCCAUGGACUGGUUCAUAGCC | |
| | GUCUGUUAUGUgGGCCUUACGUACCGAUAGACCACUGGUGCAA | |
| | AUAGAGAGCUAUCUUAGC | |
| FIG. 1C 180 bp | AUUACCACUCGACACCGAAGUAUGUUCCGCGGAGUACGCGUGU | 66 |
| sensor | gGACCAAGUCGACACGGAUGAGUGUUUCCUUCUAUCCGCGUCA | |
| | UUGUUGGCAUAUGCGACGGCCAUGGACUGGUUCAUAGCCGUCU | |
| | GUUAUGUgGGCCUUACGUACCGAUAGACCACUGGUGCAAAUAG | |
| | AGAGCUAUCUUAGCAUUACGCAACCGCGCCCACCCUUAACGAU | |
| | CAUGUCGACG | |
| FIG. 1C 360 bp | GGUUCACUCGCCGACAGUCAGGCAGGUUUCUUCUACUUUCAAA | 67 |
| sensor | GCUGUUGCUACGUGAGUAUUCCUCAGACUCUGCCACAACCGGC | |
| | AUCUAUUACCACUCGACACCGAAGUAUGUUCCGCGGAGUACGC | |
| | GUGUgGACCAAGUCGACACGGAUGAGUGUUUCCUUCUAUCCGC | |
| | GUCAUUGUUGGCAUAUGCGACGGCCAUGGACUGGUUCAUAGCC | |
| | GUCUGUUAUGUgGGCCUUACGUACCGAUAGACCACUGGUGCAA | |
| | AUAGAGAGCUAUCUUAGCAUUACGCAACCGCGCCCACCCUUAA | |
| | CGAUCAUGUCGACGAGAUCAGUUACUUAUUUCCCGCUACACCU | |
| | GCUCUCCGCGGAGCACUUCGGCGCGCAAAUCCUGCCUGAGGGU | |
| | UUGGUAGACAAGGCAGGG | |
| FIG. 2 Exon 6-8 | AUGGGAUCCAUAAUUCCCCCACCACCUCCCAUAUGUCCAGAUUC | 68 |
| trigger | UCUUGAUGAUGCUGAUGCUUUGGGAAGUAUGUUAAUUUCAUGG | |
| | UACAUGAGUGGCUAUCAUACUGGCUAUUAUAUGGAAAUGCUGG | |
| | CAUAGAGCAGCAC | |
| FIG. 2 Exon 6-7-8 | AUGGGAUCCAUAAUUCCCCCACCACCUCCCAUAUGUCCAGAUUC | 69 |
| trigger | UCUUGAUGAUGCUGAUGCUUUGGGAAGUAUGUUAAUUUCAUGG | |
| | UACAUGAGUGGCUAUCAUACUGGCUAUUAUAUGGGUUUUAGAC | |
| | AAAAUCAAAAAGAAGGAAGGUGCUCACAUUCCUUAAAUAUAAG | |
| | GAGAAAUGCUGGCAUAGAGCAGCAC | |
| FIG. 2 72 bp sensor | AGCAUUUCUCCUUAUAUUGAAGGAAUGCGAGCACCUUGGCAUA | 70 |
| | UGCGACGGCCAUGGACUGGUUCAUAGCCGUCUGUUAUGUCCUU | |
| | CUUUUUGAUUUUGUCUAAAACCCAUAUAAUA | |
| FIG. 2 90 bp sensor | CUCUAUGCCAGCAUUUCUCCUUAUAUUGAAGGAAUGCGAGCAC | 71 |
| | CUUGGCAUAUGCGACGGCCAUGGACUGGUUCAUAGCCGUCUGU | |
| | UAUGUCCUUCUUUUUGAUUUUGUCUAAAACCCAUAUAAUAGCC | |
| | AGUAUG | |
| FIG. 2 102 bp sensor | GUGCUGCUCUAUGCCAGCAUUUCUCCUUAUAUUGAAGGAAUGC | 72 |
| | GAGCACCUUGGCAUAUGCGACGGCCAUGGACUGGUUCAUAGCC | |
| | GUCUGUUAUGUCCUUCUUUUUGAUUUUGUCUAAAACCCAUAUA | |
| | AUAGCCAGUAUGAUAGCC | |
| SP019 sensor stem- | ATGCGACGGCCATGGACTGGTTCATAGCCGTCTGTT | 73 |
| loop element (DNA | | |
| cognate of RNA | | |
| sequence) | | |
| SP019 sensor element | [45 OR 90 CONSECUTIVE NUCLEOTIDES REVERSE | 74 |
| containing | COMPLEMENTARY TO TDP043 TRIGGER SEQUENCE]-- | |
| stem-loop | ATGCGACGGCCATGGACTGGTTCATAGCCGTCTGTT--[45 OR 90 | |
| | CONSECUTIVE NUCLEOTIDES REVERSE COMPLEMENTARY TO | |
| | TDP043 TRIGGER SEQUENCE] | |
| SP127 out of frame | AGTGATAATTTGATAACG | 75 |
| sequence containing | | |
| stop codons (DNA | | |
| cognate of RNA | | |
| sequence) | | |
| SP478 RNA sensor | AGCATTTATTGTAGCAAAGAGTGGGTAGGGACAGGAGCTCTAGG | 76 |
| element containing | ACTGGCCAGTGGGTGTTCTAGAGGCCAGCTGGGGTTGGAAGACA | |
| 90 bp APOA2 hy- | ATATGCGACGGCCATGGACTGGTTCATAGCCGTCTGTTGGTCTGG | |
| bridization region, | ACACTTCACTGGGTGGCAGGCTGTGTTCCAAGTTCCACGAAATAG | |
| stem loop, and 90 bp | CTCAAGAAGTTAACCAGTTCCGTTCCAGCCTTCTTGAT | |
| APOA3 hybridization | | |
| region (DNA cognate | | |
| of RNA sequence) | | |
| TP043 trigger se- | AGGCACAGACACCAAGGACAGAGACGCTGGCTAGGCCGCCCTCC | 77 |
| quence/APOA2 full | CCACTGTTACCAACATGAAGCTGCTCGCAGCAACTGTGCTACTCC | |
| transcript (DNA | TCACCATCTGCAGCCTTGAAGGAGCTTTGGTTCGGAGACAGGCAA | |
| cognate of RNA | AGGAGCCATGTGTGGAGAGCCTGGTTTCTCAGTACTTCCAGACCG | |
| sequence) | TGACTGACTATGGCAAGGACCTGATGGAGAAGGTCAAGAGCCCA | |
| | GAGCTTCAGGCCGAGGCCAAGTCTTACTTTGAAAAGTCAAAGGA | |
| | GCAGCTGACACCCCTGATCAAGAAGGCTGGAACGGAACTGGTTA | |
| | ACTTCTTGAGCTATTTCGTGGAACTTGGAACACAGCCTGCCACCC | |
| | AGTGAAGTGTCCAGACCATTGTCTTCCAACCCCAGCTGGCCTCTA | |
| | GAACACCCACTGGCCAGTCCTAGAGCTCCTGTCCCTACCCACTCT | |
| | TTGCTACAATAAATGCTGAATGAATCCA | |
| Negative control | CCAGGAGGCACCATAAGTGCCTGAGACTGAGGTGCCCCAGTAAT | 78 |
| trigger sequence | CGCCGGGTAGGGAGCCCGCTGGTTGAGTGGTGACCTTTACTCGTC | |
| | TTCTCACATTATGGAAGCCCTTCCGGGAATGGTGGACGATACACT | |
| | GTCTATCTGCAGTTGCCCATCTATGGCCACGGATGGGGATAGATT | |
| | GGCGCAATAGGGTCCGCGTCTCGCTAAAAGTTACCCGAGGCGTTA | |
| | ACATTAAACTATAGCCGATAGTTATCACAGGCAAGCGGGTAGTTG | |
| | GTTGAGAGATAGCGAGGCCTGCCGTCCGGAAAGATAAGCTCCCG | |
| | TGTACAACCAACATCAATAGACTCGATGAGCGCGCCAAGAATTT | |
| | AAG | |

Also provided are kits for practicing the methods described in the present disclosure. In general, subject kits may contain a sensor RNA as described above. The sensor RNA may be contained in a lipid nanoparticle or the sensor RNA may be within a recombinant vector as described above, e.g., an AAV vector. In some cases, the kit further contains an ADAR protein or a coding sequence thereof. When the kit contains a coding sequence of the ADAR it may be in a recombinant vector as described above. When the kit contains the ADAR protein or the coding sequence thereof, the ADAR protein may be any ADAR protein described above. The sensor RNA and the coding sequence of the ADAR protein may be contained on the same recombinant vector or different recombinant vectors.

In some cases, the kit may further contain a positive or negative control. The positive control may be in the form of a biological sample containing the target RNA, a sensor RNA containing an edited codon (e.g., a stop codon that has been edited to be a non-stop codon or a start codon edited to be a non-start codon or a non-start codon edited to be a start codon) or a sensor RNA containing the nucleotide sequence of the target RNA. The negative control may be in the form of a biological sample that does not contain the target RNA.

A subject kit can include any combination of components for performing the methods of the present disclosure. The components of a subject kit can be present as a mixture or can be separate entities. In some cases, components are present as a lyophilized mixture. In some cases, the components are present as a liquid mixture. Components of a subject kit can be in the same or separate containers, in any combination.

The subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a remote site.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations are to be accounted for. Unless indicated otherwise, parts are parts by weight, molecular weight is weight average molecular weight, temperature is in degrees Centigrade, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### Example 1

### Introduction

The abundance of available single-cell transcriptomics data allows a cell's type and state to be inferred from its RNA signature. However, methods to detect and act upon RNA transcripts in living mammalian cells have historically been lacking. Such a tool can enable the identification and manipulation of specific cell types in living organisms as research tools, the selective ablation of pathogenic cells in human patients (e.g., cancer or autoimmune cells), and the cell type-specific delivery of in vivo gene therapies. Towards this goal, systems for RNA sensing have been described that leverage the RNA-editing abilities of adenosine deaminases acting on RNA (ADARs). These sensors take advantage of ADAR's ability to edit adenosines (A) to inosines (I), altering a stop codon upstream (e.g. 5′) of a payload and allowing for the translation of a downstream (e.g. 3′) payload conditioned upon the expression of a specific RNA transcript (referred to as “trigger”, “input”, or “target” hereafter).

In some embodiments, these designs display selectivity for specific sequence motifs in the trigger, analogous to CRISPR-Cas enzymes' requirements for a protospacer-adjacent motif (PAM) and here termed stop codon removal by ADAR motifs (“SCRAM”). When 5′-UAG-3′ is used as the stop codon, an appropriate SCRAM in the trigger is 5′-CCA-3′. In the presence of a trigger, dsRNA forms around the stop codon, forming a C:A mismatch in the UAG:CCA pairing that increases editing efficiency. The dsRNA recruits ADAR, and the catalytic domain localizes to the site of the mismatch, editing the A in the UAG stop codon to I (read as guanine so that the codon UIG is read as UGG, encoding tryptophan instead of a stop) enabling translation of the payload.

Generally, a CCA subsequence within a transcript functions as the SCRAM, which base-pairs with UAG except for a C:A mismatch. Other subsequences may function as SCRAMs with reduced efficiency. Similarly to a PAM, the SCRAM requirement limits our ability to fully optimize sensor design (e.g., by prioritizing trigger regions with features such as low secondary structure or by empirically testing sensor candidates via tiling the trigger) and can prohibit the sensing of short transcripts or sub-sequences that lack this motif.

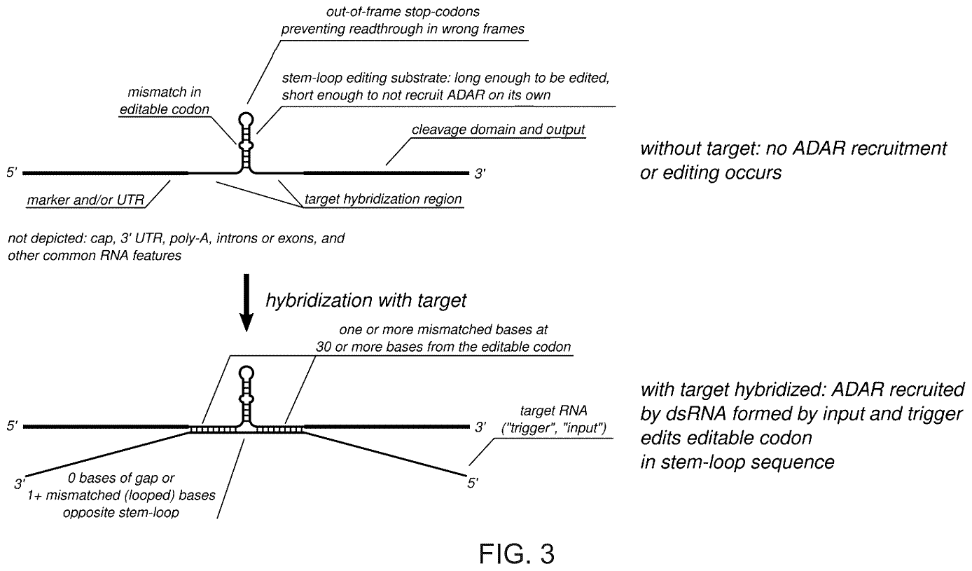

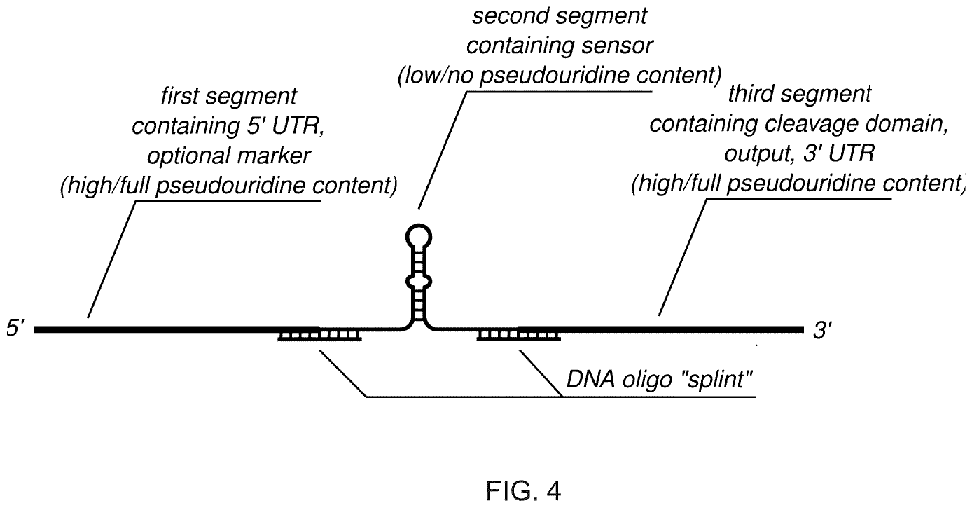

Here, SCRAM-less RNA sensors that are completely unconstrained by a particular sequence requirement are engineered. Unlike linear sensors, this more modular system for RNA sensing using adenosine deaminases acting on RNA (“ModulADAR”) instead relies on ADAR editing of a stem-loop sequence derived from natural ADAR substrates. In ModulADAR, ADAR recruitment and editing are separated into two modules: dsRNA is formed with an input and trigger to recruit ADAR, but editing occurs in a stem-loop of the sensor, which alone is not enough to recruit ADAR (FIG. 3). Stem-loops are screened and identified that achieve similar signal as linear sensors, showing that the choice of stem-loop can be orthogonal to the sensor sequence. Finally, ModulADAR is applied towards screening for better, unconstrained sensor sequences and sensing otherwise indiscernible splice isoforms. Overall, ModulADAR will empower more sensitive and broadly useful RNA sensors for basic science and therapeutic applications.

### Results

### Development and Characterization of Stem-Loop Sensors

Experiments on linear sensors (e.g. sensors having x base pairs require a specific SCRAM motif (e.g., CCA, GCA, UCA, or UUA) in the endogenous transcript of interest. The SCRAM was thoroughly mapped and it was determined it is approximated with the rule (U/G/C)NA.

However, it was posited that using substrates optimized through evolution are a better starting point better RNA sensors. To achieve SCRAM-less RNA sensors, ADAR's two functionalities were separated (RNA binding via the dsRNA binding domain, RBD, and editing by the catalytic deaminase domain, DD). Here, ADAR is recruited by dsRNA formed by a trigger and sensor, but editing occurs in the stem-loop which either natively contains a stop codon or has been modified to contain one. Sensors were designed to be reverse complementary to a synthetic trigger sequence, except for a central stop-containing step-loop. Stem-loops were screened for those that are capable of mediating sensor activation, including stem-loops of varying length as natural editing sites contain RBD-binding stems that were contemplated to cause baseline signaling in the context of ModulADAR. Utilized stem-loop sequences were derived from endogenous ADAR substrates that are natively edited by the DD with efficiencies over 99%, and then shortened them so that ADAR does not edit them unless the trigger was present to form dsRNA with the sensor.

It was found that a GluR-B stem-loop modified to enhance editing (“syn GluR-B”) as well as native GluR-B and GABRA3-derived stem-loops of various lengths functioned in ModulADAR sensors (FIG. 1A). Consistent with our hypothesis, it was generally found that shorter stem-loops perform better: these are long enough to be bound by the ADAR DD domain, yet short enough to avoid ADAR recruitment independent of sensor:trigger dsRNA. Critically, sensors using GABRA-3 derived stem-loops achieve signals similar to linear sensors (FIG. 1B).

It was considered utilizing these stem-loops to sense an unrelated transcript. Sensors were created for the 3′ UTR of mouse Bdnf transcripts. The relative signal-to-noise ratio is similar between this transcript and the previously tested synthetic transcript (FIG. 1B). This suggests that the choice of stem-loop can be orthogonal to the sensor sequence and that the sensor sequence and stem-loop can be optimized independently of one another. To better inform such engineering efforts, rules for sensor length were explored. A minimal sensor length of −90 bp was identified, consistent with ADAR's documented activity dimerizing with a footprint of roughly 50 bases (FIG. 1C). Superior activation was achieved with longer sensors of 180-360 bp.

The lack of a SCRAM requirement affords ModulADAR significantly more flexibility in choice of target sequence than linear sensors. This is important given observations that sensor sequences are currently difficult to predict a priori: for example, it was observed that out of three linear sensors designed around different SCRAMs in the mouse Bdnf 3′ UTR, one was functional. ModulADAR was taken advantage of to design additional sensor candidates optimized either for regions of low secondary structure or (since regions of low structure are more likely to be AT-rich) GC-content (40-60%).

ModulADAR also has a unique advantage in sensing short transcripts or subsequences such as exons, enabling the discrimination of many splice isoforms inaccessible through linear RNA sensors. While 95% of genes undergo some form of alternative splicing, the short length of exons (on average <200 bp) makes it unlikely that a given exon will contain a SCRAM. One such exon is the 54 bp exon 7 of SMN2 (survival of motor neuron 2). Alternative splicing of SMN2 to include exon 7 is of great clinical interest, as this isoform can rescue defects in SMN1 (survival of motor neuron 1) that cause spinal muscular atrophy (SMA). Sensors were designed for exon 7, finding that a shorter sensor (72 bp) allowed for optimal discrimination between isoforms (FIG. 2). This is likely because longer sensors can bind to longer regions in adjacent exons present in all splice isoforms and thus not contributing to discrimination.

TABLE 1
Sequences used in FIG. 1

| | | |
|---|---|---|
| | | SEQ ID |
| Name | Sequence | NO: |

FIG. 1A

| | | |
|---|---|---|
| Trigger sequence: | CCCUGCCUUGUCUACCAAACCCUCAGGCAGGAUUUGCGCGCCGA | 50 |
| | AGUGCUCCGCGGAGAGCAGGUGUAGCGGGAAAUAAGUAACUGA | |
| | UCUCGUCGACAUGAUCGUUAAGGGUGGGCGCGGUUGCGUAAUG | |
| | CUAAGAUAGCUCUCUAUUUGCACCAGUGGUCUAUCGGUACGUA | |
| | AGGCCCAACAAUGACGCGGAUAGAAGGAAACACUCAUCCGUGU | |
| | CGACUUGGUCCACACGCGUACUCCGCGGAACAUACUUCGGUGUC | |
| | GAGUGGUAAUAGAUGCCGGUUGUGGCAGAGUCUGAGGAAUACU | |
| | CACGUAGCAACAGCUUUGAAAGUAGAAGAAACCUGCCUGACUG | |
| | UCGGCGAGUGAACC | |
| Linear sensor: | ACCAAGUCGACACGGAUGAGUGUUUCCUUCUAUCCGCGUCAUU | 51 |
| | GUUaGGCCUUACGUACCGAUAGACCACUGGUGCAAAUAGAGAG | |
| | CUAU | |
| Syn GlurR-B sensor | ACCAAGUCGACACGGAUGAGUGUUUCCUUCUAUCCGCGUCAUU | 52 |
| | GUUCCGUUUAGGUGGGUGGAAUAGUAAUACAAAGUAUCCCACC | |
| | UACCCAGACGUgGGCCUUACGUACCGAUAGACCACUGGUGCAAA | |
| | UAGAGAGCUAU | |
| 15 bp GluR-B sensor | ACCAAGUCGACACGGAUGAGUGUUUCCUUCUAUCCGCGUCAUU | 53 |
| | GUacCAUUUAGGUGGGUGGGCUAACCACCUACCCaGAUGccUgGG | |
| | CCUUACGUACCGAUAGACCACUGGUGCAAAUAGAGAGCUAU | |
| 18 bp GluR-B sensor | ACCAAGUCGACACGGAUGAGUGUUUCCUUCUAUCCGCGUCAUU | 54 |
| | GUacCAUUUAGGUGGGUGGAAUGCUAAAUCCCACCUACCCaGAU | |
| | GccUgGGCCUUACGUACCGAUAGACCACUGGUGCAAAUAGAGAG | |
| | CUAU | |
| 36 bp GABRA3 sensor | ACCAAGUCGACACGGAUGAGUGUUUCCUUCUAUCCGCGUCAUU | 55 |
| | GUAUGCGACGGCCAUGGACUGGUUCAUAGCCGUCUGUUUgGGC | |
| | CUUACGUACCGAUAGACCACUGGUGCAAAUAGAGAGCUAU | |
| 45 bp GABRA3 sensor | ACCAAGUCGACACGGAUGAGUGUUUCCUUCUAUCCGCGUCAUU | 56 |
| | GUUGGCAUAUGCGACGGCCAUGGACUGGUUCAUAGCCGUCUGU | |
| | UAUGUgGGCCUUACGUACCGAUAGACCACUGGUGCAAAUAGAG | |
| | AGCUAU | |

FIG. 1B

| | | |
|---|---|---|
| Trigger sequence: | UGGAUUUAUGUUGUAUAGAUUAUAUUGAGACAAAAUUAUCUA | 57 |
| | UUUGUAUAUAUACAUAACAGGGUAAAUUAUUCAGUUAAGAAA | |
| | AAAUAAUUUUAUGAACUGCAUGUAUAAAUGAAGUUUAUACAG | |
| | UACAGUGGUUCUACAAUCUAUUUAUUGGACAUAUCCAUGACCU | |
| | GAAAGGAAACAGUCAUUUGCGCACAACUUUAAAAGUCUGCAUU | |
| | ACAUUCCUCGAUAAUGUUGUGGUUUGUUGCCGUUGCCAAGAAU | |
| | UGAAAACAAAAAGUUUAAAAAAAAUAAUAAUAAAUUGCAUGC | |
| | UGCUUUAAUUGUGAAUUGAUAAUAAACUGUCCCUCUUUCAGAA | |
| | AACAGAUUAAAAAAACAAAAAACAAAAAAAAAAAAACAAAAAA | |
| | CAAAAACAAAAAUUGGAACCAAAACAUUCCGUUUACAUUUUAG | |
| | ACACUAAGUAUCUUCGUUCUUGUUAGUACUCUGUUUUACUGCU | |
| | UUCGACUUCUGAUAGCGUUGGAAUUAAAACAAUGUCAAGGUGC | |
| | UGUUGUCAUUGCUUUACUGGCGUAAGGGACGGGGAAUGGGAGG | |
| | GGUAGAUUUCUGUUUGUUUUGUGUUUUAUUUUGUUUGUUUGU | |
| | UUGUUUUGUUUUUUAGUUCCACCCGGAGUAGGGAUGGAGAAAA | |
| | UUUCUUCACUAUCCAUUCUGGUUGAUAAAGCGUUACAUUUGUA | |
| | UGUUGUAAAGAUGUUUGCAAAAUCCAAUCAGAUGACUGGAAAA | |
| | CAAAUAAAAAUUAAGGCAACUGAAUAAAAUGCUCACACUCCAC | |
| | UGCCCAUGAUGUAUCUCCCUGGUCCCCCUCAGCUCACUCUUCUG | |
| | GCAUGGGUCAGGGAAAAUUGCUUUUAUUGGAAAGACCAGCAUU | |
| | UGUUCAAAGCAUACUCUUUCCCUCCCUCCUCCCAUUUUGGUCCC | |
| | UUCUUUUUGUUUUGUUUUAAGAAAGAAAAUUAAGUUGCGCGCU | |
| | UUAAAAUAUUUUACUACUGCUACAAACAGAUGAACAAUGUGUG | |
| | UCAUUUUAUGACACUCAUGGAAAACAGUGAUUUUUUUUUACCC | |
| | UAAAGAAAAACAAAUAAAAAUAACCCAAAAUAUUCUUUUUUUA | |
| | AAAGGCAUAAAUAUUGGGUAAAUUGUAAUAUGGCCUAACAGUG | |
| | UUUGCAGAUAAAAGUUAUUGUAUACACCCAGAUACUUAGAUAA | |
| | GAGCAGGGAUCCACACUGCCAUUGAAAUAGGACUGAAUGGCCC | |
| | UGCGGAGGCUAAGUGGAGCUGACAUACUAUUUCCUGGCAGUGC | |
| | AGGAGGAAUUUCUGAGUGGCCAUCCUAAGGUCUAGGAUGGAGG | |
| | UGGGGAAUGGUACUUGAGACAUUCCUAAAGGAAGGCUCGGAAG | |
| | CACCCUUCAGAGCAGGCUCUGGAAUGAUGUGUCAAGUUGCUUA | |
| | GGCCUUCUGCUUUAAGUGCCUACAUUACCUAACAGUGCUCAAG | |
| | AGGUUCUCGAUUGGAGAACCACACUCAAAUCCAUUUAUAGCCU | |
| | CCAUCCCAUUUCUAAAUAAUUGUGUAUAAAGUUGGAUUAACCU | |
| | GGAGCAACUUUGGAUCCAAAUAUGGCACAGCAAUAAUGAUAUU | |
| | AAUGCAGCAUGAUGGGAAAUGUUUGCUGUGAAGAGAAUUGAU | |
| | UUGCUUUGAGCUUAGACUUCAGGAAGCCUAGGUUUUUUAUUUU | |
| | UUUAUUUUUGAGACAUUUUGGUAAAAGGAAAAAAAGAAAACA | |
| | AACAAACAAACAAACAAAACCAGAAAAAGCAUCAAAACUCAGG | |
| | CAGAAUGAGCAAUGUCUGAAAGGGCUAGAAAAACAAGACAUAG | |
| | CAAGGUGCUUUCACUGUGAAAGAGACAAGAACACAGGAGGAAA | |
| | UAUUGCUUCAGUGAAGAGCACAGACGGCUCCUGCCAAUUUAUU | |
| | ACAAGAGUCCCGUCUGUACUUUACCCUUUGGGGUUAGAAGUCA | |
| | AGUUGGAAGCCUGAAUGAAUGGACCCAAUGAGAACUAGUGUUA | |
| | AGCCCAUUUCCCUAGUCAGGUUUUUUUCAAGCGUGAAUGUGUU | |
| | AGUGGUUACUCUCCUGGGUUCCUGAGCAUCAGAAAAAAAAAAA | |
| | AAAAGAGGCAAACAAUCGCUUCAUCUUAGGAGUGGAAAGGAAA | |
| | CAGAAGUGGACGUCCGCUGUGACUCAGGGAGUGAAGAUACCAU | |
| | CAGCAAAUAGUUUCUUUUUUGUUCAUUCGUUCCUUUCGAGUUA | |
| | GCCUGUCUUUUGGAAUACCACUGAAUAUGCUGUUUUUGAAAGA | |
| | CUUCAUGUAGCAUAGAUUGUUUUGUGCCGUUUACCAAAUUAAC | |
| | CUUUGUCAUCGUUUUUUAACCUAUUCAGGAAUGCUUGGAAUAU | |
| | CUGCUCUAUGUUAACUUUUUGCAGCUUCAUUCUGAGAGACAUU | |
| | AGUCAAACAAACAAAAGGAUCCCCAUCACAAUCUUACAGUACU | |
| | GCAAGGGCCAGGUCUGUUAAUCGGCUUCACAGGAGACAUCAGC | |
| | AAUUGUGUGGUCAGUGGCUGGCUCUCUUACCCACUAAGAUACA | |
| | UCAUAGCUACAUGUUGGUGGUUUAUGUUGACCUGAGAUUUAUU | |
| | UGUUAAAAUCUCUUCUUCGUUUCUGUUCGUUCUGGUUCUGUUC | |
| | UGUUCUGUUCUGUUCUGUUUUGGUUUUAAAGUCUUGCUGUGGU | |
| | CUCUUGUUGGCAGAAAUGUUUUAUGCAUGGCAGCAGGCCUGUU | |
| | GCUUUUUUAUAGUGAUUCCCAUUGAAACUGUAAGUAAAUGUCU | |
| | GUGGCCUUGUUCUCUCUAUGGUAAAGAUAUUAUUCACCAUGUA | |
| | AAACAAGAAAAAAUAUUUAUUGUAUUUUAGUAUAUUUAUAUA | |
| | AUUAUGUUAUUGAAAAAAUUGGCAUUAAAACUUAACCACAUCA | |
| | GAAGCCUAUUGUAAAUACAGGUUCUAUUUAAGUGUACCAAUUA | |
| | ACAUAUAAUAUAUGUUUUAAAUA | |
| Syn GlurR-B sensor | CAAACAUCUUUACAACAUACAAAUGUAACGCUUUAUCAACCAG | 58 |
| | AAUCCGUUUAGGUGGGUGGAAUAGUAAUACAAAGUAUCCCACC | |
| | UACCCAGACGUGGAUAGUGAAGAAAUUUUCUCCAUCCCUACUC | |
| | CGGGUGGAACUA | |
| 15 bp GluR-B sensor | CAAACAUCUUUACAACAUACAAAUGUAACGCUUUAUCAACCAG | 59 |
| | AAacCAUUUAGGUGGGUGGGCUAACCACCUACCCaGAUGccUGGA | |
| | UAGUGAAGAAAUUUUCUCCAUCCCUACUCCGGGUGGAACUA | |
| 36 bp GABRA3 sensor | CAAACAUCUUUACAACAUACAAAUGUAACGCUUUAUCAACCAG | 60 |
| | AAAUGCGACGGCCAUGGACUGGUUCAUAGCCGUCUGUUUGGAU | |
| | AGUGAAGAAAUUUUCUCCAUCCCUACUCCGGGUGGAACUA | |
| 45 bp GABRA3 sensor | CAAACAUCUUUACAACAUACAAAUGUAACGCUUUAUCAACCAG | 61 |
| | AAUGGCAUAUGCGACGGCCAUGGACUGGUUCAUAGCCGUCUGU | |
| | UAUGUGGAUAGUGAAGAAAUUUUCUCCAUCCCUACUCCGGGUG | |
| | GAACUA | |

FIG. 1C

| | | |
|---|---|---|
| Trigger sequence: | CCCUGCCUUGUCUACCAAACCCUCAGGCAGGAUUUGCGCGCCGA | 62 |
| | AGUGCUCCGCGGAGAGCAGGUGUAGCGGGAAAUAAGUAACUGA | |
| | UCUCGUCGACAUGAUCGUUAAGGGUGGGCGCGGUUGCGUAAUG | |
| | CUAAGAUAGCUCUCUAUUUGCACCAGUGGUCUAUCGGUACGUA | |
| | AGGCCCAACAAUGACGCGGAUAGAAGGAAACACUCAUCCGUGU | |
| | CGACUUGGUCCACACGCGUACUCCGCGGAACAUACUUCGGUGUC | |
| | GAGUGGUAAUAGAUGCCGGUUGUGGCAGAGUCUGAGGAAUACU | |
| | CACGUAGCAACAGCUUUGAAAGUAGAAGAAACCUGCCUGACUG | |
| | UCGGCGAGUGAACC | |
| 60 bp sensor | AUGAGUGUUUCCUUCUAUCCGCGUCAUUGUUGGCAUAUGCGAC | 63 |
| | GGCCAUGGACUGGUUCAUAGCCGUCUGUUAUGUgGGCCUUACG | |
| | UACCGAUAGACCACUGGUG | |
| 90 bp sensor | ACCAAGUCGACACGGAUGAGUGUUUCCUUCUAUCCGCGUCAUU | 64 |
| | GUUGGCAUAUGCGACGGCCAUGGACUGGUUCAUAGCCGUCUGU | |
| | UAUGUgGGCCUUACGUACCGAUAGACCACUGGUGCAAAUAGAG | |
| | AGCUAU | |
| 102 bp sensor | GUGUgGACCAAGUCGACACGGAUGAGUGUUUCCUUCUAUCCGC | 65 |
| | GUCAUUGUUGGCAUAUGCGACGGCCAUGGACUGGUUCAUAGCC | |
| | GUCUGUUAUGUgGGCCUUACGUACCGAUAGACCACUGGUGCAA | |
| | AUAGAGAGCUAUCUUAGC | |
| 180 bp sensor | AUUACCACUCGACACCGAAGUAUGUUCCGCGGAGUACGCGUGU | 66 |
| | gGACCAAGUCGACACGGAUGAGUGUUUCCUUCUAUCCGCGUCA | |
| | UUGUUGGCAUAUGCGACGGCCAUGGACUGGUUCAUAGCCGUCU | |
| | GUUAUGUgGGCCUUACGUACCGAUAGACCACUGGUGCAAAUAG | |
| | AGAGCUAUCUUAGCAUUACGCAACCGCGCCCACCCUUAACGAU | |
| | CAUGUCGACG | |
| 360 bp sensor | GGUUCACUCGCCGACAGUCAGGCAGGUUUCUUCUACUUUCAAA | 67 |
| | GCUGUUGCUACGUGAGUAUUCCUCAGACUCUGCCACAACCGGC | |
| | AUCUAUUACCACUCGACACCGAAGUAUGUUCCGCGGAGUACGC | |
| | GUGUgGACCAAGUCGACACGGAUGAGUGUUUCCUUCUAUCCGC | |
| | GUCAUUGUUGGCAUAUGCGACGGCCAUGGACUGGUUCAUAGCC | |
| | GUCUGUUAUGUgGGCCUUACGUACCGAUAGACCACUGGUGCAA | |
| | AUAGAGAGCUAUCUUAGCAUUACGCAACCGCGCCCACCCUUAA | |
| | CGAUCAUGUCGACGAGAUCAGUUACUUAUUUCCCGCUACACCU | |
| | GCUCUCCGCGGAGCACUUCGGCGCGCAAAUCCUGCCUGAGGGU | |
| | UUGGUAGACAAGGCAGGG | |

TABLE 2
Sequences used in FIG. 2

| | | SEQ ID |
|---|---|---|
| Name | Sequence | NO: |
| Exon 6-8 | AUGGGAUCCAUAAUUCCCCCACCACCUCCCAUAUGUCCAGAUUCU- | 68 |
| trigger | CUUGAU- | |
| | GAUGCUGAUGCUUUGGGAAGUAUGUUAAUUUCAUGGUACAUGAGUG | |
| | GCUAUCAUACUGGCUAUUAUAUGGAAAUGCUGGCAUAGAGCAGCAC | |
| Exon 6-7-8 | AUGGGAUCCAUAAUUCCCCCACCACCUCCCAUAUGUCCAGAUUCU- | 69 |
| trigger | CUUGAU- | |
| | GAUGCUGAUGCUUUGGGAAGUAUGUUAAUUUCAUGGUACAUGAGUG | |
| | GCUAUCAUACUGGCUAUUAUAUGGGUUUUAGACAAAAUCAAAAA- | |
| | GAAGGAAGGUGCUCACAU- | |
| | UCCUUAAAUAUAAGGAGAAAUGCUGGCAUAGAGCAGCAC | |
| 72 bp sensor | AGCAUUUCUCCUUAUAUUGAAGGAAUGCGAGCAC- | 70 |
| | CUUGGCAUAUGCGACGGCCAUG- | |
| | GACUGGUUCAUAGCCGUCUGUUAUGUCCUUCUUUUUGAUUUUGUCU | |
| | AAAACCCAUAUAAUA | |
| 90 bp sensor | CUCUAUGCCAGCAUUUCUCCUUAUAUUGAAGGAAUGCGAGCAC- | 71 |
| | CUUGGCAUAUGCGAC- | |
| | GGCCAUGGACUGGUUCAUAGCCGUCUGUUAUGUCCUUCUUUUUGAU | |
| | UUUGUCUAAAACCCAUAUAAUAGCCAGUAUG | |
| 102 bp sensor | GUGCUGCUCUAUGCCAGCAUUUCUCCUUAUAUUGAAGGAAUGCGAG- | 72 |
| | CAC- | |
| | CUUGGCAUAUGCGACGGCCAUGGACUGGUUCAUAGCCGUCUGUUAU | |
| | GUCCUUCUUUUUGAUUUUGUCU- | |
| | AAAACCCAUAUAAUAGCCAGUAUGAUAGCC | |

### Discussion

Here, RNA sensors with improved, sequence-unconstrained ModulADAR sensors were described. It was shown that such sensors allow for similar signal-to-noise as prior-generation, linear sensors, and that they can exceed the performance of linear sensors by allowing for better optimization of the sensor sequence. We observed that designing sensors for transiently transfected targets is easier than designing sensors for endogenous genes, as a subset of such sensors are functional in this context. However, the ability to rationally design sensors or empirically tile sensors along a transcript of interest enables the better design of sensors for endogenous genes. Additionally, the described sensors were tested using ADAR overexpression unless otherwise stated, as it was observed with much lower activation without ADAR overexpression. However, since it was observed that ModulADAR can be less dependent on ADAR overexpression than linear sensors, the development of stem-loops better at recruiting ADAR allow for functional with endogenous ADAR (e.g. in the absence of ADAR supplementation).

ModulADAR's ability to effectively tile a transcript in search of suitable subsequences to detect provides for designing sensors based on features such as presence or absence of secondary structure, local level of GC-content, sites of RNA binding-proteins, sites of RNA:RNA interactions.

In further aspects or embodiments, RNA sensors may enable the development of new research tools as well as “smart” gene therapies that allow for cell type-specific expression of therapeutic cargo. We anticipate that ModulADAR may better enables these tools due to its lack of a SCRAM requirement. Furthermore, ModulADAR is uniquely suited for developing tools targeting short transcripts, including highly structured viral RNAs (which may have short regions suitable for sensor design) and ncRNAs such as those associated with cancer. This may better enable the identification and tracking of such cells in a research setting, as well as the development of genetic therapies that selectively ablate virus-infected or cancer cells. ModulADAR is similarly suited for detecting alternative splicing, which regulates a wide variety of cellular processes including immune cell differentiation and cancer metastasis. As even ubiquitously expressed genes undergo cell type-specific splicing, this allows for the discrimination of cell types based on isoform expression.

Notably, the ability of splice isoforms to both cause and ameliorate disease has motivated the development of splice-switching antisense-oligonucleotide (ASO) that work by selectively interfering with the splicing machinery. These include nusinersen (for the treatment of SMA) and several ASOs for the treatment of Duchenne muscular dystrophy (e.g., eteplisersen, golodirsen). With the development of more recent small molecules that can also selectively affect splicing (e.g., Risdiplam for the treatment of SMA), it has become apparent that more convenient and inexpensive small molecules can also selectively affect splicing. While risdiplam was discovered via the overexpression of an intron-exon cassette fused to a reporter gene (a strategy widely used for investigating alternative splicing), ModulADAR is better suited for screening both small molecules and ASOs as it allows for isoform sensing in the context of the native pre-mRNA. ModulADAR may also be better suited for developing patient-specific, “N of 1” therapies in patient-derived cells, as it does not require the overexpression of an exon-intron reporter. This approach cancan be extended to the wide range of cancers and hereditary disorders caused by or potentially rescued by alternative splicing, including Dravet syndrome (caused by the inclusion of a “poison” exon in the sodium channel SCN1A) and Hutchinson-Gilford progeria syndrome (caused by defective splicing in the lamin gene LMNA).

### Example 2

It was contemplated that introducing out of frame stop codons into RNA sensors as described herein would decrease non-specific activation (e.g. activation by nonspecific sequences) of the RNA sensor.

Accordingly, vectors encoding the RNA sensor sequences SP019 (comprising SEQ ID NO: 74, containing a 45 or 90 bp nucleotide sequence hybridizing to a trigger RNA, followed by a stem-loop sequence, followed by a 45 or 90 bp nucleotide sequence hybridizing to a trigger RNA, followed by a luciferase coding sequence), SP047 (containing UAG flanked on either side by 45 or 90 bp nucleotide sequences hybridizing to a trigger RNA followed by a luciferase coding sequence), and SP127 (which comprises the same sequence as SP019 except having the out of frame stop codon sequence of SEQ ID NO: 75 inserted before the luciferase coding sequence) were constructed, alongside vectors encoding the trigger sequence (SEQ ID NO: 77) and a negative control sequence (SEQ ID NO: 78).

To assess on-target and off-target activation of the RNA sensors, each of SP019, SP047, and SP127 vectors were co-transfected into cultured cell lines (HEK293 cells, 293FT cells, and HEK293-JumpIn cells) alongside either a vector encoding the trigger sequence or a vector encoding the negative control sequence using conventional transient transfection procedures, the transfected cells were incubated, and the production of luciferase in the cultured cell lines was assessed by luminometry.

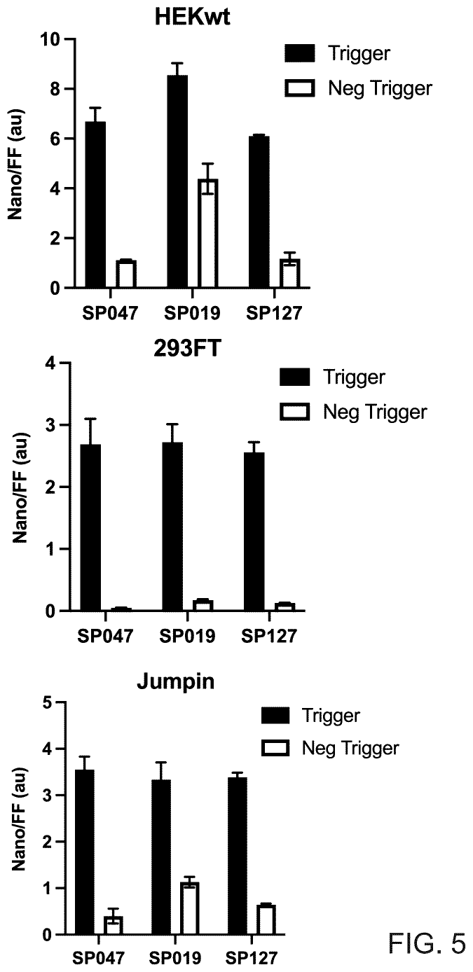

The results are presented in FIG. 5, which shows graphs of luminescence versus each transfection condition where either SP047, SP019, or SP127 vector was co-transfected with the trigger sequence (“trigger”) or the negative control sequence (“neg trigger”) into either HEK293 cells (“HEKwt”), 293FT cells (“293FT”), and HEK293-JumpIn cells (“Jumpin”). In each cell line measured, the non-specific activation of the sensor (“negative trigger”) was decreased between the SP019 and SP127 conditions, indicating that the introduction of the out of frame stop codon in SP127 reduced non-specific activation of the sensor.

### Example 3

It was contemplated that mismatches might be tolerated or beneficial when introduced to regions that hybridize to target or predetermined RNAs of sensor RNAs as described herein.

Accordingly, a pooled experiment was devised to test the editing efficiency of editable codons within sensor RNAs as described herein when mismatches were introduced to the regions that hybridize to target or predetermined RNAs of the sensor RNAs. For this experiment, a vector encoding the RNA sensor sequence SP478 (comprising SEQ ID NO: 76, containing a 90 nucleotide sequence hybridizing to a trigger RNA, followed by a stem-loop sequence, followed by a 90 nucleotide sequence hybridizing to a trigger RNA) was constructed, alongside a vector encoding the trigger sequence (SEQ ID NO: 77). A vector encoding a negative control RNA sensor sequence (comprising the stem loop in SEQ ID NO: 76 flanked on each side by 90 nucleotide random sequence) was also constructed. Two pools of sequences were derived from each of the vector encoding the SP478 RNA sensor sequence and the vector encoding the negative-control RNA sensor sequence: (1) a first pool, wherein 3 nucleotide long tiled mismatches were introduced throughout the length of the upstream (e.g. 5′) and downstream (e.g. 3′) nucleotide sequences flanking the stem-loop by inverting the sequence of 3 nucleotides at a time (e.g. converting a first sequence 5′ GCA 3′ that normally base-pairs with 3′ CGU 5′ to 5′ CGU 3′); and (2) a second pool, wherein 3 nucleotide long tiled inserts (“bulges”) comprising UUC (or TTC for the DNA cognate of the RNA sequence) were introduced throughout the length of the upstream (e.g. 5′) and downstream (e.g. 3′) nucleotide sequences flanking the stem-loops. The four resultant pools was then transfected into HEK293 cells using conventional transient transfection procedures alongside a vector encoding the trigger sequence, the transfected cells were incubated, and the editing at the editable codons of each of the sensor sequences encoded by the vectors was assessed by next-generation sequencing.

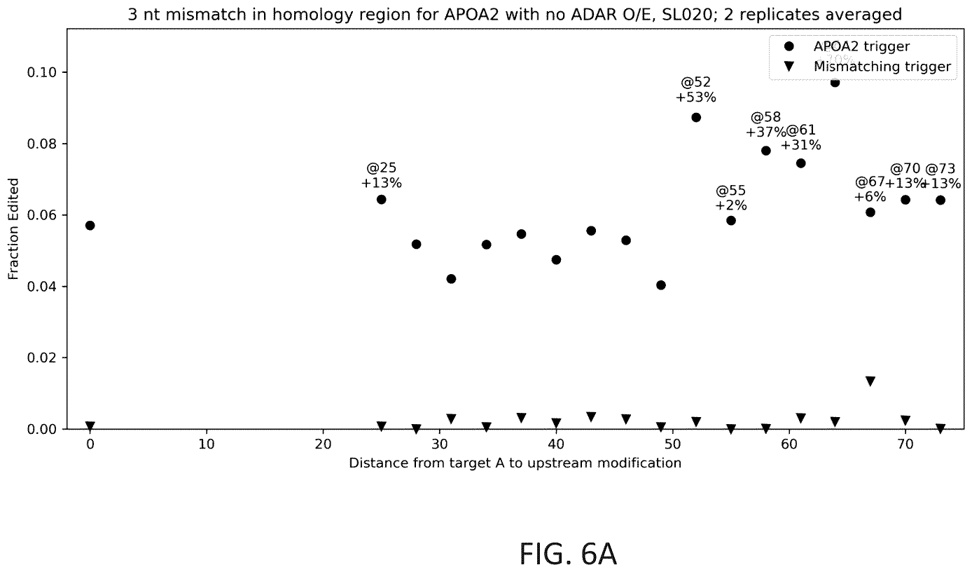

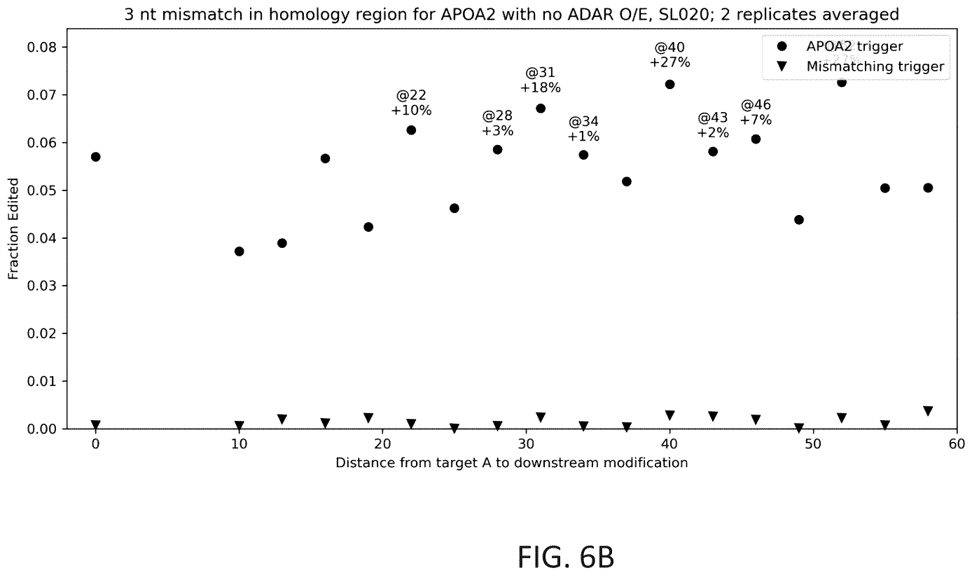

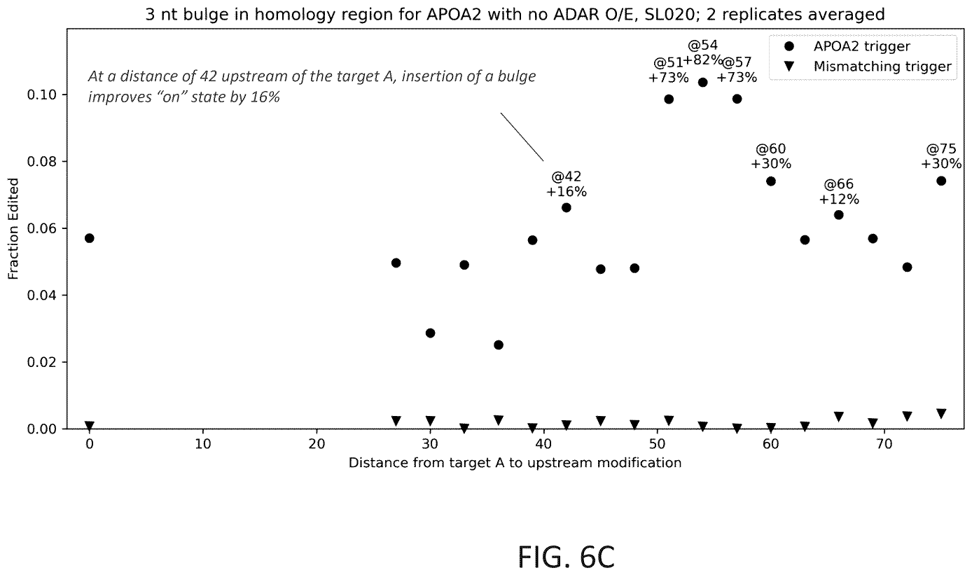

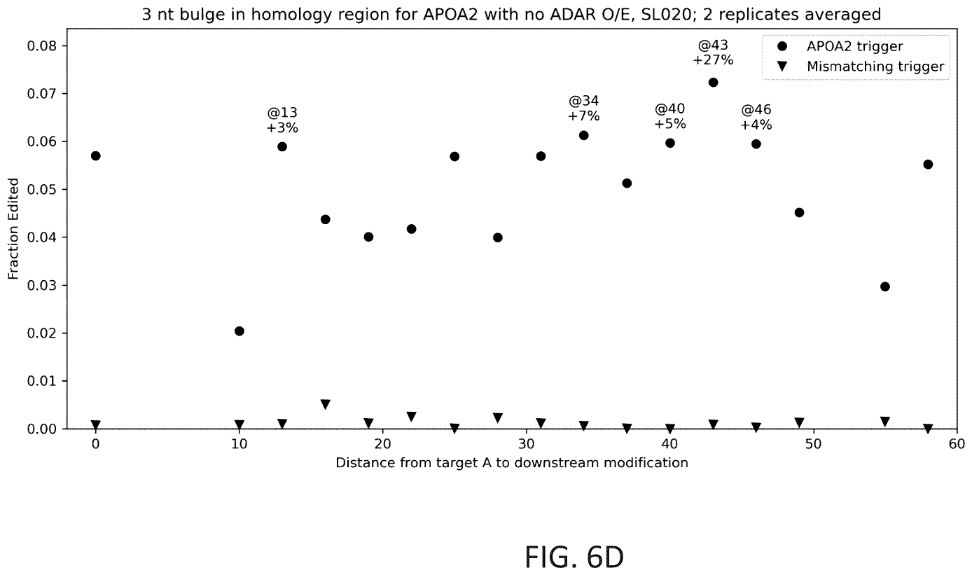

The results of this experiment are shown in FIGS. 6A, 6B, 6C, and 6D. FIGS. 6A and 6B show the results where 3 nucleotide long tiled mismatches were introduced throughout the length of the upstream (e.g. 5′) and downstream (e.g. 3′) trigger-hybridizing or non-targeting nucleotide sequences flanking the stem-loops, with FIG. 6A representing the results for mismatches introduced in the upstream trigger-hybridizing nucleotide sequences and FIG. 6B representing the results for mismatches introduced in the downstream trigger-hybridizing nucleotide sequences. FIGS. 6C and 6D show the results where 3 nucleotide long tiled inserts (“bulges”) were introduced throughout the length of the upstream (e.g. 5′) and downstream (e.g. 3′) trigger-hybridizing or non-targeting nucleotide sequences flanking the stem-loops, with FIG. 6C representing the results for inserts introduced in the upstream trigger-hybridizing nucleotide sequences and FIG. 6D representing the results for inserts introduced in the downstream trigger-hybridizing nucleotide sequences. For each of FIGS. 6A, 6B, 6C, and 6D, editing efficiency of the editable codon within the sensor construct is shown on the y-axis as fraction edited out of all sequences detected, wherein the x-axis indicates distance in nucleotides of the mismatch or insert upstream or downstream from the edited A of the editable codon of the sensor RNA. Also for each of FIGS. 6A, 6B, 6C, and 6D, editing efficiency of the editable codon for the SP478-derived RNA sensor sequence is shown in the circular data points when paired with the matching trigger (“APOA2 trigger”), whereas editing efficiency of the editable codon when co-transfected with a control is shown in the triangular data points (“mismatching trigger”). FIGS. 6A, 6B, 6C, and 6D show that mismatches and inserts are tolerated throughout the length of both the upstream and downstream sequences flanking the stem-loops of the sensor (as editing is not abrogated at any of the data points), and that in some instances (see e.g. FIG. 6C, which shows that inserts at about 40 or 42 nucleotides upstream of the stem-loop improve editing efficiency), the mismatches or inserts improve efficiency of editing at the editable codon.

In at least some of the described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” is to be interpreted as “including but not limited to,” the term “having” is to be interpreted as “having at least,” the term “includes” is to be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases need not be construed to imply that the introduction of a claim recitation by the indefinite articles “a” or “an” limits any particular claim containing such introduced claim recitation to embodiments containing solely one such recitation, even when the same claim includes the introductory phrases “one or more” or “at least one” and indefinite articles such as “a” or “an” (e.g., “a” or “an” is to be interpreted to mean “at least one” or “one or more”); the same holds true for the use of definite articles used to introduce claim recitations. In addition, even if a specific number of an introduced claim recitation is explicitly recited, those skilled in the art will recognize that such recitation is to be interpreted to mean at least the recited number (e.g., the bare recitation of “two recitations,” without other modifiers, generally signifies at least two recitations, or two or more recitations). Furthermore, in those instances where a convention analogous to “at least one of A, B, and C, etc.” is used, in general such a construction is intended in the sense one having skill in the art may understand the convention (e.g., “a system having at least one of A, B, and C” may include but not be limited to systems that have A alone, B alone, C alone, A and B together, A and C together, B and C together, or A, B, and C together, etc.). In those instances where a convention analogous to “at least one of A, B, or C, etc.” is used, in general such a construction is intended in the sense one having skill in the art may understand the convention (e.g., “a system having at least one of A, B, or C” may include but not be limited to systems that have A alone, B alone, C alone, A and B together, A and C together, B and C together, or A, B, and C together, etc.).

It will be further understood by those within the art that virtually any disjunctive word and/or phrase presenting two or more alternative terms, whether in the description, claims, or drawings, is to be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, e.g., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

Notwithstanding the appended claims, the disclosure set forth herein is also described by the following clauses:
1. A method for expressing a protein in a target cell, the method comprising:
   contacting the target cell with a sensor RNA or a vector encoding a sensor RNA comprising:
      (i) a first nucleotide sequence comprising a nucleotide sequence that hybridizes to the target RNA and a stem-loop sequence comprising one or more editable codons,
      (ii) a second nucleotide sequence encoding a first cleavage domain, and
      (iii) a third nucleotide sequence encoding an output protein;
   wherein:
   the target RNA is present in the target cell, and
   the stem-loop sequence is defined by a sequence selected from the group consisting of SEQ ID NO:10, SEQ ID NO: 11, and SEQ ID NO: 12.
2. The method of clause 1, wherein the editable codon is a stop codon, a start codon, or an AUA codon.
3. The method of clauses 1 or 2, wherein the editable codon comprises one or more bases that is mismatched with a sequence within the stem-loop opposite the one or more editable codons.
4. The method of any one of clauses 1-3, wherein the cleavage domain is a 2A self-cleaving domain
5. The method of clause 4, wherein the 2A self-cleaving domain is selected from the group of T2A, P2A, E2A, and F2A.
6. The method of any one of clauses 1-5, wherein the output protein is selected from a fluorescent protein, a genomic modification protein, a transcription factor, a killing factor, a toxin, an antigen, a T cell receptor, a chimeric antigen receptor, a therapeutic protein, and an enzyme.
7. The method of any one of clauses 1-6, wherein the target RNA is associated with a disease, condition, cell type, or tissue.
8. The method of any one of clauses 1-7, wherein the target RNA is encoded by a gene fusion, a splice variant, a gene variant comprising a single nucleotide polymorphism, or a multi-nucleotide variant.
9. The method of clause 7, wherein the output protein treats the disease, or condition.
10. The method of any one of clauses 1-9, wherein the nucleotide sequence that hybridizes to the target RNA hybridizes to two or more non-contiguous sequences within a single target RNA.
11. The method of any one of clauses 1-10, wherein the combining with the target cell comprises contacting the target cell with a lipid nanoparticle comprising the sensor RNA.
12. The method of any one of clauses 1-11 wherein the combining with the target cell comprises contacting the target cell with an adeno-associated virus (AAV) comprising the sensor RNA wherein the sensor RNA is contained in an AAV vector.
13. The method of any one of clauses 1-12, wherein the sensor RNA further comprises a nucleotide sequence encoding a second nucleotide sequence that hybridizes to a second target RNA wherein the sequences of the first and second target RNAs are different.
14. The method of any one of clauses 1-13, further comprising:
   (i) a fourth nucleotide sequence comprising a second cleavage domain wherein the fourth nucleotide sequence precedes the first nucleotide sequence and
   (ii) a fifth nucleotide sequence comprising a nucleotide sequence encoding a marker protein wherein the fifth nucleotide sequence precedes the fourth nucleotide sequence.
15. The method of any one of clauses 1-13, further comprising combining the target cell with an adenosine deaminase acting on RNA (ADAR) protein or a coding sequence thereof.
16. The method of clause 15, wherein the ADAR protein is selected from the group consisting of ADAR2, ADAR1p110, ADAR1p150, a modified ADAR comprising an ADAR deaminase domain, and an RNA motif binding domain.
17. The method of any one of clauses 1-16, further comprising assaying for the presence of the output protein.
18. The method of clause 17, wherein the assaying comprises using microscopy, flow cytometry, immunoblotting, plate reader, or a combination thereof.
19. The method of any one of clauses 1-18, wherein combining comprises administering to a patient.
20. The method of any one of clauses 1-19, wherein the target RNA comprises one or more bases mismatched opposite of the stem-loop sequence.
21. The method of any one of clauses 1-20, wherein the target RNA comprises five or more base mismatches opposite of the stem-loop sequence.
22. The method of any one of clauses 1-21, wherein the target RNA comprises ten or more base mismatches opposite of the stem-loop sequence.
23. The method of any one of clauses 1-22, wherein the nucleotide sequence that hybridizes to the target RNA comprises one or more bases mismatch 25 or more nucleotides upstream (e.g. 5′) or downstream (e.g. 3′) of the editable codon.
24. The method of any one of clauses 1-23, wherein the nucleotide sequence that hybridizes to the target RNA comprises three or more base mismatches 25 or more nucleotides upstream (e.g. 5′) or downstream (e.g. 3′) of the editable codon.
25. The method of any one of clauses 1-24, wherein the nucleotide sequence that hybridizes to the target RNA comprises ten or more base mismatches 25 or more nucleotides upstream (e.g. 5′) or downstream (e.g. 3′) of the editable codon.
26. A method for expressing a protein in a target cell, the method comprising:
   combining the target cell with a sensor RNA comprising:
      (i) a first nucleotide sequence comprising a sensor nucleotide sequence that hybridizes to the target RNA, and a stem-loop sequence comprising one or more editable codons,
      (ii) a second nucleotide sequence encoding a first cleavage domain, and
      (iii) a third nucleotide sequence encoding an output protein;
   wherein:
   the target RNA is present in the target cell, and
   the target RNA comprises one or more base mismatch opposite of the stem-loop sequence.
27. The method of clause 26, wherein the target RNA comprises five or more base mismatches opposite of the stem-loop sequence.
28. The method of clauses 26 or 27, wherein the target RNA comprises ten or more base mismatches opposite of the stem-loop sequence.
29. The method of any one of clauses 26-28, wherein the editable codon is a stop codon, a start codon, or an AUA codon.
30. The method of any one of clauses 26-29, wherein the editable codon comprises one or more bases that are mismatched with a sequence within the stem-loop opposite the one or more editable codons.
31. The method of any one of clauses 26-30, wherein the cleavage domain is a 2A self-cleaving domain.
32. The method of clause 31, wherein the 2A self-cleaving domain is selected from the group of T2A, P2A, E2A, and F2A.
33. The method of any one of clauses 26-32, wherein the output protein is selected from a fluorescent protein, a genomic modification protein, a transcription factor, a killing factor, a toxin, an antigen, a T cell receptor, a chimeric antigen receptor, a therapeutic protein, and an enzyme.
34. The method of any one of clauses 26-33, wherein the target RNA is associated with a disease, condition, cell type, or tissue.
35. The method of any one of clauses 26-34, wherein the target RNA is encoded by a gene fusion, a splice variant, a gene variant comprising a single nucleotide polymorphism, or a multi-nucleotide variant.
36. The method of clause 34, wherein the output protein treats the disease or condition.
37. The method of any one of clauses 26-36, wherein the nucleotide sequence that hybridizes to the target RNA hybridizes to two or more non-contiguous sequences within a single target RNA.
38. The method of any one of clauses 26-33, wherein the combining with the target cell comprises contacting the target cell with a lipid nanoparticle comprising the sensor RNA.
39. The method of any one of clauses 26-37, wherein the combining with the target cell comprises contacting the target cell with an adeno-associated virus (AAV) comprising the sensor RNA wherein the sensor RNA is contained in an AAV vector.
40. The method of any one of clauses 26-39, wherein the sensor RNA further comprises a nucleotide sequence encoding a second nucleotide sequence that hybridizes to a second target RNA wherein the sequences of the first and second target RNAs are different.
41. The method of any one of clauses 26-40, further comprising:
   (i) a fourth nucleotide sequence comprising a second cleavage domain wherein the fourth nucleotide sequence precedes the first nucleotide sequence and
   (ii) a fifth nucleotide sequence comprising a nucleotide sequence encoding a marker protein wherein the fifth nucleotide sequence precedes the fourth nucleotide sequence.
42. The method of any one of clauses 26-41, further comprising combining the target cell with an adenosine deaminase acting on RNA (ADAR) protein or a coding sequence thereof.
43. The method of clause 42, wherein the ADAR protein is selected from the group consisting of ADAR2, ADAR1p110, ADAR1p150, a modified ADAR comprising an ADAR deaminase domain and an RNA motif binding domain.
44. The method of any one of clauses 26-43, further comprising assaying for the presence of the output protein.
45. The method of clause 43, wherein the assaying comprises using microscopy, flow cytometry, immunoblotting, a plate reader, or a combination thereof
46. The method of any one of clauses 26-45, wherein combining comprises administering to a patient.
47. The method of any one of clauses 26-46, wherein the nucleotide sequence that hybridizes to the target RNA comprises one or more base mismatches 25 or more nucleotides upstream (e.g. 5′) or downstream (e.g. 3′) of the editable codon.
48. The method of any one of clauses 26-47, wherein the nucleotide sequence that hybridizes to the target RNA comprises three or more base mismatches 25 or more nucleotides upstream (e.g. 5′) or downstream (e.g. 3′) of the editable codon.
49. The method of any one of clauses 26-48, wherein the nucleotide sequence that hybridizes to the target RNA comprises ten or more base mismatches 25 or more nucleotides upstream (e.g. 5′) or downstream (e.g. 3′) of the editable codon.
50. The method of any one of clauses 26-49, wherein the sensor RNA, the stem-loop sequence, or the region that hybridizes to the target RNA comprises one or more stop codons that are out of frame of the editable codon.
51. The method of any one of clauses 26-50, wherein the stem-loop sequence comprises two or more stop codons that are out of frame of the editable codon.
52. The method of clause 51, wherein the two or more stop codons that are out of frame are defined by CUAAAUAAA (SEQ ID NO: 13).
53. A method for expressing a protein in a target cell, the method comprising:
   contacting the target cell with a sensor RNA comprising:
      (66) a first nucleotide sequence comprising a sensor nucleotide sequence that hybridizes to the target RNA and a stem-loop sequence comprising one or more editable codons,
      (ii) a second nucleotide sequence encoding a first cleavage domain, and
      (iii) a third nucleotide sequence encoding an output protein;
   wherein:
   the target RNA is present in the target cell, and
   the nucleotide sequence that hybridizes to the target RNA comprises one or more mismatch 25 or more nucleotides upstream (e.g. 5′) or downstream (e.g. 3′) of the editable codon.
54. The method of clause 53, wherein the nucleotide sequence that hybridizes to the target RNA comprises three or more base mismatches 25 or more nucleotides upstream (e.g. 5′) or downstream (e.g. 3′) of the editable codon.
55. The method of clause 53 or 54, wherein the nucleotide sequence that hybridizes to the target RNA comprises ten or more base mismatches 25 or more nucleotides upstream (e.g. 5′) or downstream (e.g. 3′) of the editable codon.
56. The method of any one of clauses 53-55, wherein the editable codon is a stop codon, a start codon, or an AUA codon.
57. The method of any one of clauses 53-56, wherein the editable codon comprises one or more bases that are mismatched with a sequence within the stem-loop opposite the one or more editable codons.
58. The method of any one of clauses 53-57, wherein the cleavage domain is a 2A self-cleaving domain
59. The method of clause 58, wherein the 2A self-cleaving domain is selected from the group of T2A, P2A, E2A, and F2A.
60. The method of any one of clauses 53-59, wherein the output protein is selected from a fluorescent protein, a genomic modification protein, a transcription factor, a killing factor, a toxin, an antigen, a T cell receptor, a chimeric antigen receptor, a therapeutic protein, and an enzyme.
61. The method of any one of clauses 53-60, wherein the target RNA is associated with a disease, condition, cell type, or tissue.
62. The method of any one of clauses 53-61, wherein the target RNA is encoded by a gene fusion, a splice variant, a gene variant comprising a single nucleotide polymorphism, or a multi-nucleotide variant.
63. The method of clause 61, wherein the output protein treats the disease or condition.
64. The method of any one of clauses 53-63, wherein the nucleotide sequence that hybridizes to the target RNA hybridizes to two or more non-contiguous sequences within a single target RNA.
65. The method of any one of clauses 53-64, wherein the combining with the target cell comprises contacting the target cell with a lipid nanoparticle comprising the sensor RNA.
66. The method of any one of clauses 53-64, wherein the combining with the target cell comprises contacting the target cell with an adeno-associated virus (AAV) comprising the sensor RNA wherein the sensor RNA is contained in an AAV vector.
67. The method of any one of clauses 53-66, wherein the sensor RNA further comprises a nucleotide sequence encoding a sensor nucleotide sequence that hybridizes to a second target RNA wherein the sequences of the first and second target RNAs are different.
68. The method of any one of clauses 53-67, further comprising:
   (i) a fourth nucleotide sequence comprising a second cleavage domain wherein the fourth nucleotide sequence precedes the first nucleotide sequence and
   (ii) a fifth nucleotide sequence comprising a nucleotide sequence encoding a marker protein wherein the fifth nucleotide sequence precedes the fourth nucleotide sequence.
69. The method of any one of clauses 53-68, further comprising combining the target cell with an adenosine deaminase acting on RNA (ADAR) protein or a coding sequence thereof.
70 The method of clause 69, wherein the ADAR protein is selected from the group consisting of ADAR2, ADAR1p110, ADAR1p150, a modified ADAR comprising an ADAR deaminase domain and an RNA motif binding domain.
71. The method of any one of clauses 53-70, further comprising assaying for the presence of the output protein
72. The method of clause 71, wherein the assaying comprises using microscopy, flow cytometry, immunoblotting, a plate reader, or a combination thereof.
73. The method of any one of clauses 53-72, wherein combining comprises administering to a patient.
74. The method of any one of clauses 53-73, wherein the stem-loop sequence comprises one or more stop codons that are out of frame of the editable codon.
75. The method of any one of clauses 53-74, wherein the stem-loop sequence comprises two or more stop codons that are out of frame of the editable codon.
76. The method of clause 75, wherein the two or more stop codons that are out of frame are defined by CUAAAUAAA (SEQ ID NO: 13).
77. The method of any one of clauses 53-76, wherein the target RNA comprises one or more base mismatches opposite of the stem-loop sequence.
78. The method of any one of clauses 53-77, wherein the target RNA comprises five or more base mismatches opposite of the stem-loop sequence.
79. The method of any one of clauses 53-78, wherein the target RNA comprises ten or more base mismatches opposite of the stem-loop sequence.
80. A method for expressing a protein in a target cell, the method comprising:
   contacting the target cell with a sensor RNA comprising:
      (i) a first nucleotide sequence comprising a sensor nucleotide sequence that hybridizes to the target RNA and a stem-loop sequence comprising one or more editable codons,
      (ii) a second nucleotide sequence encoding a first cleavage domain, and
      (iii) a third nucleotide sequence encoding an output protein;
   wherein:
   the target RNA is present in the target cell, and
   the stem-loop sequence comprises one or more stop codons that are out of frame of the editable codon.
81. The method of clause 80, wherein the stem-loop sequence comprises two or more stop codons that are out of frame of the editable codon.
82. The method of clause 81, wherein the two or more stop codons that are out of frame are defined by CUAAAUAAA (SEQ ID NO: 13).
83. The method of any one of clauses 80-82, wherein the editable codon is a stop codon, a start codon, or an AUA codon.
84. The method of any one of clauses 80-83, wherein the editable codon comprises one or more bases that are mismatched with a sequence within the stem-loop opposite the one or more editable codons.
85. The method of any one of clauses 80-84, wherein the cleavage domain is a 2A self-cleaving domain
86. The method of clause 85, wherein the 2A self-cleaving domain is selected from the group of T2A, P2A, E2A, and F2A.
87. The method of any one of clauses 80-86, wherein the output protein is selected from a fluorescent protein, a genomic modification protein, a transcription factor, a killing factor, a toxin, an antigen, a T cell receptor, a chimeric antigen receptor, a therapeutic protein, and an enzyme.
88. The method of any one of clauses 80-87, wherein the target RNA is associated with a disease, condition, cell type, or tissue.
89. The method of any one of clauses 80-88, wherein the target RNA is encoded by a gene fusion, a splice variant, a gene variant comprising a single nucleotide polymorphism, or a multi-nucleotide variant.
90. The method of clause 88, wherein the output protein treats the disease or condition.
91. The method of any one of clauses 80-90, wherein the nucleotide sequence that hybridizes to the target RNA hybridizes to two or more non-contiguous sequences within a single target RNA.
92. The method of any one of clauses 80-91, wherein the contacting to the target cell comprises contacting the target cell with a lipid nanoparticle comprising the sensor RNA.
93. The method of any one of clauses 80-91, wherein the combining with the target cell comprises contacting the target cell with an adeno-associated virus (AAV) comprising the sensor RNA wherein the sensor RNA is contained in an AAV vector.
94. The method of any one of clauses 80-93, wherein the sensor RNA further comprises a nucleotide sequence encoding a second nucleotide sequence that hybridizes to a second target RNA wherein the sequences of the first and second target RNAs are different.
95. The method of any one of clauses 80-94, further comprising:
   (i) a fourth nucleotide sequence comprising a second cleavage domain wherein the fourth nucleotide sequence precedes the first nucleotide sequence and
   (ii) a fifth nucleotide sequence comprising a nucleotide sequence encoding a marker protein wherein the fifth nucleotide sequence precedes the fourth nucleotide sequence.
96. The method of any one of clauses 80-95, further comprising combining the target cell with an adenosine deaminase acting on RNA (ADAR) protein or a coding sequence thereof.
97. The method of clause 96, wherein the ADAR protein is selected from the group consisting of ADAR2, ADAR1p110, ADAR1p150, a modified ADAR comprising an ADAR deaminase domain and an RNA motif binding domain.
98. The method of any one of clauses 80-97, further comprising assaying for the presence of the output protein.
99. The method of clause 98, wherein the assaying comprises using microscopy, flow cytometry, immunoblotting, a plate reader, or a combination thereof.
100. The method of any one of clauses 80-99, wherein combining comprises administering to a patient.
101. The method of any one of clauses 80-100, wherein the target RNA comprises one or more base mismatches opposite of the stem-loop sequence.
102. The method of any one of clauses 80-101, wherein the target RNA comprises five or more base mismatches opposite of the stem-loop sequence.
103. The method of any one of clauses 80-102, wherein the target RNA comprises ten or more base mismatches opposite of the stem-loop sequence.
104. The method of any one of clauses 80-103, wherein the nucleotide sequence that hybridizes to the target RNA comprises one or more base mismatches 25 or more nucleotides upstream (e.g. 5′) or downstream (e.g. 3′) of the editable codon.
105. The method of any one of clauses 80-104, wherein the nucleotide sequence that hybridizes to the target RNA sequence comprises three or more base mismatches.
106. The method of any one of clauses 80-105, wherein the nucleotide sequence that hybridizes to the target RNA comprises ten or more base mismatches 25 or more nucleotides upstream (e.g. 5′) or downstream (e.g. 3′) of the editable codon.
107. A recombinant vector comprising the sensor RNA of any one of the preceding clauses, wherein the sensor RNA is operably linked to a promoter.
108. The recombinant vector of clause 107, wherein the promoter is a CMV promoter.
109. The recombinant vector of clause 107 or 108, wherein the recombinant vector comprises a mmPeg10 3′ UTR and a mmPeg10 5′ UTR.
110. A method of generating a pseudouridine-containing sensor RNA, the method comprising:
   combining:
      (i) a first segment comprising:
         (ia) a first nucleotide sequence comprising a nucleotide sequence encoding a marker protein, and
         (ib) a second nucleotide sequence comprising a first cleavage domain,
         wherein the first segment comprises one or more pseudouridines;
      (ii) a second segment comprising:
         a third nucleotide sequence comprising a sensor nucleotide sequence that hybridizes to the target RNA and a stem-loop sequence comprising one or more editable codons,
         wherein the second segment does not comprise a pseudouridine; and
      (iii) a third segment comprising:
         (iiia) a fourth nucleotide sequence encoding a first cleavage domain, and
         (iiib) a fifth nucleotide sequence encoding an output protein,
         wherein the third segment comprises one or more pseudouridines.
111. The method of clause 110, wherein the first segment comprises all pseudouridines in place of uridines.
112. The method of clauses 110 or 111, wherein the third segment comprises all pseudouridines in place of uridines.
113. The method of any one of clauses 110-112, wherein the combining comprises DNA oligo-mediated splint ligation.
114. The method of clause 113, wherein the DNA oligo-mediated splint ligation comprises:
   (a) annealing a first DNA oligo to the first segment and the second segment,
   (b) annealing a second DNA oligo to the second segment and the third segment, and
   (c) ligating the first segment, the second segment, and the third segment using a ligase.

1-25. (canceled)

26. A composition, comprising:
a sensor RNA comprising:
(i) a first nucleotide sequence comprising: (1) a first sequence region that is hybridizable to a sequence of a target RNA; and (2) a stem-loop sequence comprising an editable codon;
(ii) a second nucleotide sequence encoding an output protein; and
(iii) (1) one or more stop codons that are out of frame of the editable codon; or (2) a second sequence region hybridizable to an additional sequence of the target RNA, wherein the second sequence region comprises one or more base mismatches opposite the additional sequence of the target RNA.

27. The composition of claim 26, further comprising a cell comprising the target RNA.

28. The composition of claim 27, wherein the first sequence region of the sensor RNA is hybridized to a hybridizable sequence of the target RNA.

29. The composition of claim 28, wherein the hybridizable second sequence region of the sensor RNA is hybridized to the additional sequence of the target RNA, wherein the hybridizable second sequence region comprises one or more base mismatches opposite the additional sequence of the target RNA.

30. The composition of claim 26, wherein the sensor RNA comprises one or more stop codons that are out of frame of the editable codon.

31. The composition of claim 30, wherein the one or more stop codons that are out of frame of the editable codon are 5′ to the second nucleotide sequence encoding an output protein.

32. The composition of claim 26, wherein the editable codon comprises a stop codon, a start codon, or an AUA codon.

33. The composition of claim 26, wherein the editable codon is in the stem of the stem-loop sequence and wherein the editable codon is mismatched with its base-pairing partner in the stem.

34. The composition of claim 26, wherein the second sequence region hybridizable to the additional sequence of the target RNA comprises at least about 75% to about 95% sequence identity opposite the additional sequence of the target RNA.

35. The composition of claim 26, wherein the one or more base mismatches of the second sequence region are 25 or more nucleotides 5′ to the editable codon or 25 or more nucleotides 3′ to the editable codon.

36. The composition of claim 26, wherein the first sequence region comprises a sequence that is 5′ to the stem-loop sequence.

37. The composition of claim 26, wherein the first sequence region comprises a sequence that is 3′ to the stem-loop sequence.

38. The composition of claim 26, wherein the first sequence region comprises a sequence that is 5′ to the stem-loop sequence and a sequence that is 3′ to the stem-loop sequence.

39. The composition of claim 26, wherein the sensor RNA further comprises a 5′ untranslated region (UTR) 5′ to the first nucleotide sequence or a 3′ UTR 3′ to the second nucleotide sequence.

40. The composition of claim 39, wherein the sensor RNA further comprises a 5′ UTR, and wherein the 5′ UTR is selected from the group consisting of: a Hs Peg10 5′ UTR, a mmPeg10 5′ UTR, a HsPNMA1 5′ UTR, a mmPNMA1 5′ UTR, a HsPNMA3 5′ UTR, a mmPNMA3 5′ UTR, a HsMAOP1 5′ UTR, a mmMAOP1 5′ UTR, a HsPNMA5 5′ UTR, a mmPNMA5 5′ UTR, a HsRTL1 5′ UTR, a mmRTL1 5′ UTR, a HsZCCHC12 5′ UTR, a mmZCCHC12 5′ UTR, a HsASPRV1 5′ UTR, a mmADPRV1 5′ UTR, a HsARC1 5′ UTR, and a mmARC1 5′ UTR.

41. The composition of claim 39, wherein the sensor RNA further comprises a 3′ UTR, and wherein the 3′ UTR is selected from the group consisting of: a Hs Peg10 3′ UTR, a mmPeg10 3′ UTR, a HsPNMA1 3′ UTR, a mmPNMA1 3′ UTR, a HsPNMA3 3′ UTR, a mmPNMA3 3′ UTR, a HsMAOP1 3′ UTR, a mmMAOP1 3′ UTR, a HsPNMA5 3′ UTR, a mmPNMA5 3′ UTR, a HsRTL1 3′ UTR, a mmRTL1 3′ UTR, a HsZCCHC12 3′ UTR, a mmZCCHC12 3′ UTR, a HsASPRV1 3′ UTR, a mmADPRV1 3′ UTR, a HsARC1 3′ UTR, and a mmARC1 3′ UTR.

42. The composition of claim 26, wherein the stem-loop sequence comprises a sequence that is at least 80% identical to a sequence selected from the group consisting of: SEQ ID NO: 10, SEQ ID NO: 11, and SEQ ID NO: 12.

43. The composition of claim 26, wherein the sensor RNA encodes a cleavage domain.

44. The composition of claim 43, wherein the cleavage domain is a self-cleaving domain between the first nucleotide sequence and the second nucleotide sequence.

45. The composition of claim 26, wherein the output protein is selected from a fluorescent protein, a genomic modification protein, a transcription factor, a killing factor, a toxin, an antigen, a T cell receptor, a chimeric antigen receptor, a therapeutic protein, and an enzyme.

46. The composition of claim 26, wherein the target RNA is associated with a disease, a condition, a cell type, or a tissue.

47. The composition of claim 26, wherein the sensor RNA comprises one or more pseudouridine or N1-methyl-pseudouridine.

48. A method for expressing a protein, comprising contacting a cell comprising the target RNA with the composition of claim 26, a vector encoding the composition, or a lipid nanoparticle (LNP) comprising the composition.

49. The method of claim 23, wherein the contacting comprises contacting the cell with a vector encoding the composition, wherein the vector is selected from an adeno-associated viral vector (AAV), a retroviral vector, a herpesviral vector, a lentiviral vector, or an adenoviral vector, or with an LNP comprising the composition.

50. The method of claim 23, wherein the contacting comprises administering the composition, the vector, or the LNP to a patient.

51. The method of claim 23, further comprising contacting the cell with an adenosine deaminase acting on RNA (ADAR) protein or an engineered or variant ADAR protein, or a nucleic acid molecule encoding the ADAR protein.
